(19) United States
(12) Patent Application Publication
(10) Pub. No.: US 2014/0031368 A1
(43) Pub. Date: Jan. 30, 2014

(54) SELECTIVE HDAC INHIBITORS

(71) Applicants: Ronald Breslow, New York, NY (US); Paul A. Marks, Washington, CT (US); K. G. Abhilash, New York, NY (US); Jianing Wang, San Diego, CA (US)

(72) Inventors: Ronald Breslow, New York, NY (US); Paul A. Marks, Washington, CT (US); K. G. Abhilash, New York, NY (US); Jianing Wang, San Diego, CA (US)

(73) Assignees: THE TRUSTEES OF COLUMBIA UNIVERSITY IN THE CITY OF NEW YORK, New York, NY (US); SLOAN-KETTERING INSTITUTE FOR CANCER RESEARCH, New York, NY (US)

(21) Appl. No.: 13/937,128

(22) Filed: Jul. 8, 2013

Related U.S. Application Data
(63) Continuation of application No. 13/699,234, now abandoned.
(60) Provisional application No. 61/347,337, filed on May 21, 2010.
(60) Provisional application No. 61/402,945, filed on Sep. 7, 2010.
(60) Provisional application No. 61/442,681, filed on Feb. 14, 2011.

(51) Int. Cl. C07C 235/74; C07D 401/12; C07D 403/12; C07D 333/38; C07D 215/40; C07C 259/06

(52) U.S. Cl. CPC C07C 235/74; C07D 215/40; C07C 259/06; C07D 403/12; C07D 333/38; C07D 401/12
USPC 514/255.05; 560/44; 546/171; 560/43; 546/168; 564/153; 560/37; 544/353; 549/72; 435/375; 514/533; 514/311; 514/539; 514/575; 514/448

(57) ABSTRACT

This disclosure is related to compounds having the structure

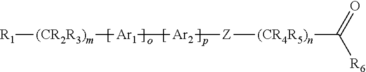

wherein Ar₁, Ar₂, R₁-R₆, Z, m, n, o, and p are defined herein. This disclosure also relates to pharmaceutical compositions comprising the above compounds and methods for their use.

This application claims priority of U.S. Provisional Applications Nos. 61/347,337, filed May 21, 2010; 61/402,945, filed Sep. 7, 2010; and 61/442,681, filed Feb. 14, 2011, the contents of which are hereby incorporated by reference.

Throughout this application, certain publications are referenced in parentheses. Full citations for these publications may be found immediately preceding the claims. The disclosures of these publications in their entireties are hereby incorporated by reference into this application in order to describe more fully the state of the art to which this invention relates.

### BACKGROUND OF THE INVENTION

To date, eighteen histone deacetylases (HDACs) have been identified in humans. Eleven HDACs (HDAC1-11) are zinc-dependent and seven HDACs, designated sirtuins 1-7, are NAD⁺-dependent (1). Aberrant activity of HDACs has been implicated in many disease states, including cancer (2). When zinc-dependent HDACs are inhibited, the levels of acetylation of certain proteins are elevated, with many resulting physiological effects. Many inhibitors of HDACs have been developed for use against cancers and other disease states. One well-known HDAC inhibitor, suberoylanilide hydroxamic acid (SAHA, Vorinostat), was approved in 2006 for human use following the results of more than 100 human trials against various forms of cancer and is currently in use. Phase I, II and III clinical trials with vorinostat as single therapy and in combination therapy with various anti-cancer agents for hematologic and solid neoplasms are ongoing. Marks & Breslow (ref. (8) describes the development of HDAC inhibitor voronistat as an anti-cancer drug; see (9) also).

While HDACs are associated with deacetylation of histones in the context of gene expression and chromatin remodeling, there is abundant evidence indicating that not all functions of HDACs are dedicated to deacetylation of histones. Rather, some HDACs have been shown to exert deacetylase activity on proteins other than histones. One such HDAC is HDAC6, a cytoplasmic, microtubule-associated deacetylase, which has been found to regulate microtubule acetylation and chemotactic cell motility (3).

HDAC6 is predominantly a cytoplasmic, microtubule-associated member of the class IIB family of histone deacetylases. HDAC6 possesses two catalytic domains, a ubiquitin-binding domain and a C-terminal zinc finger domain (4). HDAC6 catalyzes deacetylation of cytoplasmic protein substrates, such as α-tubulin, Hsp90, peroxiredoxins, and cortactin (4). HDAC6 has also been demonstrated to direct misfolded protein aggregates into aggresomes, which are major repositories formed to manage excessive levels of misfolded and aggregated protein for eventual elimination. Aggresomes are of clinical interest as they are similar to cytoplasmic inclusion bodies commonly observed in neurodegenerative diseases (5).

Haggarty et al (6) have shown that the C-terminal catalytic domain of HDAC6, the domain responsible for α-tubulin deacetylation, can be inhibited by the small-molecule inhibitor, tubacin. Haggarty et al found that the inhibition of HDAC6 with tubacin did not affect the stability of microtubules, but decreased cell motility. Given the dependence of metastasis and angiogenesis on cell movement, increasing the acetylation level of α-tubulin may be an important component to the antimetastatic and antiangiogenic activities of HDAC inhibitors (6).

Heat shock protein 90 (Hsp90) is an important chaperone protein involved in protein folding and is overexpressed in many cancer cell types (2, 7). The disruption of the folding and chaperoning functions of Hsp90 causes its client proteins to be destabilized and eventually degraded. HDAC6 is an attractive target for cancer treatment because acetylated Hsp90 has a reduced ability to perform its chaperoning function (2, 7), with consequent activation of the intrinsic pathway of apoptosis.

In general, for diseases caused by aberrant gene transcription, the most effective treatment would involve targeting only the genes relevant to the disease (2). In the context of HDAC inhibitor treatment, this would involve inhibiting only those HDAC isoforms relevant to the disease state, thereby minimizing changes not related to the disease, and possibly reducing side effects and toxicity. While SAHA combines efficacy with minimum toxicity, its inhibitory activity is not selective among the known human HDACs.

HDAC inhibitors have also been identified as a correction for cholesterol and sphingolipid transport defects in human Niemann-Pick type C disease (10).

In view of the importance of inhibiting only those HDAC isoforms relevant to a disease state, minimizing acetylation of proteins not related to the disease, and reducing side effects and toxicity, new HDAC inhibitors that are selective for specific HDACs are needed. Herein, new selective HDAC inhibitors are described.

### SUMMARY OF THE INVENTION

A compound having the structure

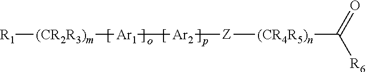

wherein
R₁ is H, halogen, —NR₇R₈, —NR₅₁—C(═O)—R₅₂, —NH—C(═O)—OR₇, —OR₇, —NO₂, —CN, —SR₇, —SO₂R₇, —CO₂R₇, CF₃, —SOR₇, —POR₇, —C(═S)R₇, —C(═O)—NR₇R₈, —CH₂—C(═O)—NR₇R₈, —C(═NR₇)R₈, —P(═O)(OR₇)(OR₈), —P(OR₇)(OR₈), —C(═S)R_{T}, C₁₋₅ alkyl, C₂₋₅ alkenyl, C₂₋₅ alkynyl, aryl, heteroaryl, or heterocyclyl,
wherein R₇, R₈, R₅₁ and R₅₂ are each, independently, H, C₁₋₅ alkyl, C₂₋₅ alkenyl, C₂₋₅ alkynyl, aryl, or heteroaryl;
   m is an integer from 0 to 5;
   R₂ and R₃ are each, independently, H, —(NH₂), —CH₂—R₉, —C(═O)OR₉, —C(═O)NR₉R₁₀, or —C(═O)R₉,
wherein
R₉ and R₁₀ are each, independently, H, C₁₋₁₀ alkyl, C₂₋₁₀ alkenyl, C₂₋₁₀ alkynyl, —(CH₂)ᵣ—OR₁₈, cycloalkyl,

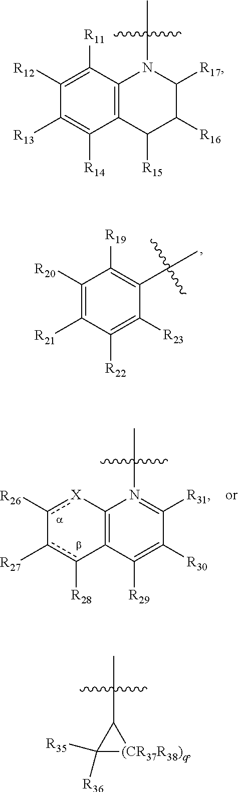

wherein
q is an integer from 1 to 6;
r is an integer from 1 to 10;
R₁₈ is H, C₁₋₁₀ alkyl, C₂₋₁₀ alkenyl, C₂₋₁₀ alkynyl, cycloalkyl, aryl, heteroaryl, or heterocyclyl;
bond α and bond β are each, independently, present or absent;
when bond α is present, X is N or CR₃₂;
when bond α is absent, X is NR₃₂ or CR₃₁R₃₂;
R₁₁, R₁₂, R₁₃, R₁₄, R₁₅, R₁₆, R₁₇, R₁₉, R₂₀, R₂₁, R₂₂, R₂₃, R₂₆, R₂₇, R₂₈, R₂₉, R₃₀, R₃₁, R₃₂, R₃₅, R₃₆, R₃₇, and R₃₈ are each, independently, H, halogen, —NO₂, —CN, —NR₂₄R₂₅, —SR₂₄, —SO₂R₂₄, —CO₂R₂₄, —OR₂₄, CF₃, —SOR₂₄, —POR₂₄, —C(═S)R₂₄, —C(═NR₂₄)R₂₅, —P(═O)(OR₂₄)(OR₂₅), —P(OR₂₄)(OR₂₅), —C(═S)R₂₄, C₁₋₁₀ alkyl, C₂₋₁₀ alkenyl, C₂₋₁₀ alkynyl, aryl, heteroaryl, or heterocyclyl;
   wherein R₂₄ and R₂₅ are each, independently, H, C₁₋₁₀ alkyl, C₂₋₁₀ alkenyl, C₂₋₁₀ alkynyl, aryl, heteroaryl, or heterocyclyl;
wherein R₂ or R₃ is other than H;
   Ar₁ and Ar₂ are each, independently, arylene or a heteroarylene other than isooxazolylene;
   o and p are each, independently, 0 or 1;
   Z is a bond, —NR₄₃—C(═O)—, or —C(—O)—NR₅₀—
wherein R₄₃ and R₅₀ are, independently, H, —(CR₄₄R₄₅)ₛ—CR₄₄R₄₅R₄₆, C₁₋₁₀ alkyl substituted by heterocyclyl, C₂₋₁₀ alkenyl, C₂₋₁₀ alkynyl, aryl, heteroaryl, or
heterocyclyl,
   wherein
   s is an integer from 1 to 10;
   R₄₄, R₄₅, and R₄₆ are each, independently, H, halogen, —NO₂, —CN, —NR₄₇R₄₈, —SR₄₇, —SO₂R₄₇, —CO₂R₄₇, —OR₄₇, CF₃, —SOR₄₇, —POR₄₇, —C(═S)R₄₇, —C(═NR₄₇)R₄₈, —P(═O)(OR₄₇)(OR₄₈), —P(OR₄₇)(OR₄₈), —C(═S)R₄₇, C₁₋₁₀ alkyl, C₂₋₁₀ alkenyl, C₂₋₁₀ alkynyl, aryl, heteroaryl, or heterocyclyl,
      wherein R₄₁ and R₄₈ are each, independently, H, C₁₋₁₀ alkyl, C₂₋₁₀ alkenyl, C₂₋₁₀ alkynyl, aryl, heteroaryl, or heterocyclyl;
wherein when Z is a bond, o is 0, p is 0, and R₂ or R₃ is C(═O)NR₉R₁₀, then R₉ and R₁₀ are each, independently, H, C₁₋₁₀ alkyl, C₂₋₁₀ alkenyl, C₂₋₁₀ alkynyl, —(CH₂)ᵣ—OR₁₈,

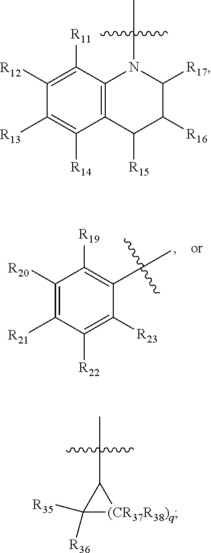

R₄ and R₅ are each, independently, H, C₁₋₁₀ alkyl, C₂₋₁₀ alkenyl, C₂₋₁₀ alkynyl, aryl, heteroaryl,

or heterocyclyl;

n is an integer and is 0 or from 2 to 10;

R₆ is —OR₄₉ or —NH—OR₄₉,
wherein R₄₉ is H, C₁₋₁₀ alkyl, C₂₋₁₀ alkenyl, C₂₋₁₀ alkynyl, aryl, heteroaryl, or heterocyclyl;

wherein if R₁ is —NR₅₁—C(═O)—R₅₂ and m=0 and Ar₁ or Ar₂ is present and is bonded directly to R₁, then Ar₁ or Ar₂, respectively, is other than triazolyl;

wherein if Z is —NR₄₃—C(═O)—, and one of o and p is 0 and the other is 1, and m=1 or 0, then R₁ is other than —NR₇R₈, —NR₅₁—C(═O)—R₅₂, or —NH—C(═O)—OR₇;

wherein if Z is —C(═O)—NR₅₀— and n=5, and R₄ and R₅ are H and R₆ is NHOH and P and O are 1 and m=0 then R₁ is other than —NH—C(═O)—OR₇;

wherein when m=0 and Ar₁ or Ar₂ is bonded directly to R₁, then R₁ is other than H;

wherein when n=0, then p=0 and o=1, and R₁ is —C(═O)NR₇R₈;

wherein each occurrence of alkyl, alkenyl, or alkynyl is unsubstituted or substituted, branched or unbranched;

wherein each occurrence of cycloalkyl, aryl, heteroaryl, heterocyclyl, arylene, or heteroarylene is unsubstituted or substituted;

or a pharmaceutically acceptable salt thereof.

A pharmaceutical composition comprising any one, or more, of the instant compounds and a pharmaceutically acceptable carrier.

A method of inhibiting the activity of a histone deactylase in a cell comprising contacting the histone deacetylase with any one, or more, of the instant compounds so as to inhibit the activity of the histone deacetylase.

A method of inhibiting the activity of a histone deacetylase 6 (HDAC6) in a cell comprising contacting the histone deacetylase 6 with any one, or more, of the instant compounds so as to inhibit the activity of the histone deacetylase 6 in the cell.

A method of increasing accumulation of acetylated alpha tubulin in a cell comprising contacting the cell with any one, or more, of the instant compounds so as to increase the accumulation of acetylated alpha-tubulin in the cell.

A method of treating a neurodegenerative disease in a subject comprising administering an effective amount of any one, or more, of the instant compounds to the subject so as to treat the disease in the subject.

A method of treating a disease associated with defective lipid transport in a subject comprising administering an effective amount of any one, or more, of the instant compounds to the subject so as to treat the disease in the subject.

### BRIEF DESCRIPTION OF THE FIGURES

FIG. 1A: Blot showing accumulation of acetylated alpha-tubulin in LNCaP cells cultured with the compound set forth in Example 3 hereinbelow. Lanes, from left to right, respectively, are: marker, untreated, DMSO, SAHA, compound at 4 μM, compound at 8 μM, compound at 12 μM, compound at 16 μM, and compound at 20 μM. GADPH used as loading control.

FIG. 1B: Blot showing no detectable accumulation of acetylated histone H3 in LNCaP cells cultured with the compound set forth in Example 3 hereinbelow. Lanes, from left to right, respectively, are: no addition, DMSO, SAHA, compound at 4 μM, compound at 20 μM, compound at 16 μM, compound at 12 μM, compound at 8 μM, and compound at 4 μM.

FIG. 2 shows anaerobic inviability and disrupted sterol metabolism of yeast ncr1Δ eaf1Δ cells.

A) Anaerobic inviability of ncr1Δ eaf1Δ and ncr1Δ yaf9Δ as a consequence of sterol auxotrophy. Five-fold dilutions of satumted, acrobically-grown cultures were plated and grown aerobically or anaerobically, respectively, for 3 days.

B) Analysis of ncr1Δ eaf1Δ strains identities a bottleneck in aerobic sterol synthesis with increased intracellular accumulation of ergosterol precursors and decreased ergosterol. Cells were grown in triplicate under, aerobic conditions in YPD at 30° C. to 100 OD units. Sterol biosynthetic intermediates were measured by GC and are expressed as a percentage of total sterols. *P<0.05, two-tailed Student's t-test comparison of ncr1Δ eaf1Δ cells to control, ncr1Δ or eaf1Δ strains.

C) Sensitivity of ncr1Δ eaf1Δ to fluconazole and nystatin. Five-fold dilutions of saturated, aerobically-grown cultures were grown aerobically at 30° C. for 2 days in the presence of the indicated drug.

FIG. 3 shows that HDAC genes are globally upregulated and pharmacologically amenable in hunum NP-C fibroblasts.

A) qRT-PCR indicates that the majority of the eleven HDAC genes are upregulated in fibroblasts derived from three patients with NP-C disease (NPC-26, NPC-2, NPC-29). *P<0.05, two-tailed Student's t-test for each NP-C fibroblast relative to the control fibroblast.

B) qRT-PCR indicates that treatment of NPC-26 fibroblasts with a HDAC inhibitor (SABA) targets this dysregulation and restores expression in the direction of control cells. *P<0.05, Student's t-test compared treated and untreated cells separately for control and NP-C fibroblasts.

FIG. 4 shows that histone deacetylase inhibition improves the cellular diagnostic criteria of NP-C disease.

A) Reduction in lysosomal accumulation of unesterified cholesterol as measured with filipin. Mutant fibroblasts were incubated for 18 h in the presence of 5 μM SAHA and stained with filipin.

B) Restoration of deficient esterification of LDL-derived cholesterol as measured by percent cholesteryl [³H]oleate formation relative to total lipids. Cells were grown for 4 d in LPDS, followed by a 24 h treatment with or without 5 μM SAHA, the last 4 h of which included LDL plus [³H]oleate. *P<0.05, treated vs. untreated cells by two-tailed Student's t-test.

C) Reduction in lysosomal accumulation of globotriaosylceramide (GL-3) as measured with verotoxin. Mutant fibroblasts were incubated for 18 h in the presence of 5 μM SAHA and stained with verotoxin. Quantification of microscopy for filipin and GL-3 (verotoxin) fluorescence performed with MetaVue is expressed as arbitrary units and demonstrates significant amelioration of both cholesterol and sphingolipids in treated cells compared to untreated cells (*P<0.05, two-tailed Student's t-test).

### DETAILED DESCRIPTION OF THE INVENTION

A compound having the structure

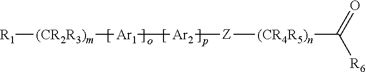

wherein

R₁ is H, halogen, —NR₇R₈, —NR₅₁—C(═O)—R₅₂, —NH—C(═O)—OR₇, —OR₇, —NO₂, —CN, —SR₇, —SO₂R₇, —CO₂R₇, CF₃, —SOR₇, —POR₇, —C(═S)R₇, —C(═O)—NR₇R₈, —CH₂—C(═O)—NR₇R₈, —C(═NR₇)R₈, —P(═O)(OR₇)(OR₈), —P(OR₇)(OR₈), —C(═S)R₇, C₁₋₅ alkyl, C₂₋₅alkenyl, C₂₋₅ alkynyl, aryl, heteroaryl, or heterocyclyl,
wherein R₇, R₈, R₅₁ and R₅₂ are each, independently, H, C₁₋₅ alkyl, C₂₋₅ alkenyl, C₂₋₅ alkynyl, aryl, or heteroaryl;

m is an integer from 0 to 5;

R₂ and R₃ are each, independently, H, —(NH₂), —CH₂—R₉, —C(═O)OR₉, —C(═O)NR₉R₁₀, or —C(═O)R₉,
wherein
R₉ and R₁₀ are each, independently, H, C₁₋₁₀ alkyl, C₂₋₁₀ alkenyl, C₂₋₁₀alkynyl, —(CH₂)ᵣ—OR₁₈, cycloalkyl,

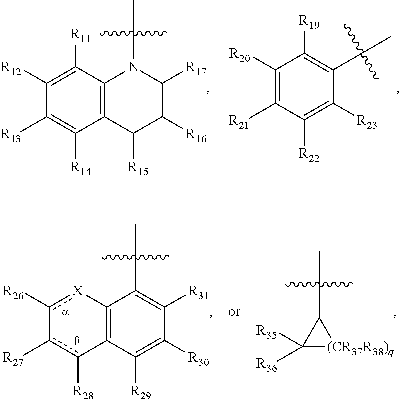

wherein
q is an integer from 1 to 6;
r is an integer from 1 to 10;
R₁₈ is H, C₁₋₁₀ alkyl, C₂₋₁₀ alkenyl, C₂₋₁₀ alkynyl, cycloalkyl, aryl, heteroaryl, or heterocyclyl;
bond α and bond β are each, independently, present or absent;
when bond α is present, X is N or CR₃₂;
when bond α is absent, X is NR₃₂ or CR₃₁R₃₂;
R₁₁, R₁₂, R₁₃, R₁₄, R₁₃, R₁₆, R₁₇, R₁₉, R₂₀, R₂₁, R₂₂, R₂₃, R₂₆, R₂₇, R₂₈, R₂₉, R₃₀, R₃₁, R₃₂, R₃₅, R₃₆, R₃₇, and R₃₈ are each, independently, H, halogen, —NO₂, —CN, —NR₂₄R₂₅, —SR₂₄, —SO₂R₂₄, —CO₂R₂₄, —OR₂₄, CF₃, —SOR₂₄, —POR₂₄, —C(═S)R₂₄, —C(═NR₂₄)R₂₅, —P(—O)(OR₂₄)(OR₂₅), —P(OR₂₄)(OR₂₅), —C(═S)R₂₄, C₁₋₁₀ alkyl, C₂₋₁₀ alkenyl, C₂₋₁₀ alkynyl, aryl, heteroaryl, or heterocyclyl;
   wherein R₂₄ and R₂₅ are each, independently, H, C₁₋₁₀ alkyl, C₂₋₁₀ alkenyl, C₂₋₁₀ alkynyl, aryl, heteroaryl, or heterocyclyl;
wherein R₂ or R₃ is other than H;

Ar₁ and Ar₂ are each, independently, arylene or a heteroarylene other than isooxazolylene;

o and p are each, independently, 0 or 1;

Z is a bond, —NR₄₃—C(═O)—, or —C(═O)—NR₅₀—
wherein R₄₃ and R₅₀ are, independently, H, —(CR₄₄R₄₅)ₛ—CR₄₄R₄₅R₄₆, C₁₋₁₀ alkyl substituted by heterocyclyl, C₂₋₁₀ alkenyl, C₂₋₁₀ alkynyl, aryl, heteroaryl, or
heterocyclyl,
   wherein
   s is an integer from 1 to 10;
   R₄₄, R₄₅, and R₄₆ are each, independently, H, halogen, —NO₂, —CN, —NR₄₇R₄₈, —SR₄₇, —SO₂R₄₇, —C₂R₄₇, —OR₄₇, CF₃, —SOR₄₇, —POR₄₇, —C(═S)R₄₇, —C(═NR₄₇)R₄₈, —P(═O)(OR₄₇)(OR₄₈), —P(OR₄₇)(OR₄₈), —C(═S)R₄₇, C₁₋₁₀ alkyl, C₂₋₁₀ alkenyl, C₂₋₁₀ alkynyl, aryl, heteroaryl, or heterocyclyl,
      wherein R₄₇ and R₄₈ are each, independently, H, C₁₋₁₀ alkyl, C₂₋₁₀ alkenyl, C₂₋₁₀ alkynyl, aryl, heteroaryl, or heterocyclyl;
wherein when Z is a bond, o is 0, p is 0, and R₂ or R₃ is C(═O)NR₉R₁₀, then R₉ and R₁₀ are each, independently, H, C₁₋₁₀ alkyl, C₂₋₁₀ alkenyl, C₂₋₁₀ alkynyl, —(CH₂)ᵣ—OR₁₈,

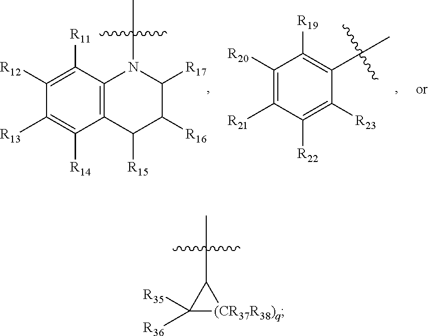

R₄ and R₅ are each, independently, H, C₁₋₁₀ alkyl, C₂₋₁₀ alkenyl, C₂₋₁₀ alkynyl, aryl, heteroaryl, or heterocyclyl;

n is an integer and is 0 or from 2 to 10;

R₆ is —OR₄₉ or —NH—OR₄₉,
wherein R₄₉ is H, C₁₋₁₀ alkyl, C₂₋₁₀ alkenyl, C₂₋₁₀ alkynyl, aryl, heteroaryl, or heterocyclyl;

wherein if R₁ is —NR₅₁—C(—O)—R₅₂ and m=0 and Ar₁ or Ar₂ is present and is bonded directly to R₁, then Ar₁ or Ar₂, respectively, is other than triazolyl;

wherein if Z is —NR₄₃—C(═O)—, and one of o and p is 0 and the other is 1, and m=1 or 0, then R₁ is other than —NR₇R₈, —NR₅₁—C(═O)—R₅₂, or —NH—C(═O)—OR₇;

wherein if Z is —C(═O)—NR₅₀— and n=5, and R₄ and R₅ are H and R₆ is NHOH and P and O are 1 and m=0 then R₁ is other than —NH—C(═O)—OR₇;

wherein when m=0 and Ar₁ or Ar₂ is bonded directly to R₁, then R₁ is other than H;

wherein when n=0, then p=0 and o=1, and R₁ is —C(═O)NR₇R₈;

wherein each occurrence of alkyl, alkenyl, or alkynyl is unsubstituted or substituted, branched or unbranched;

wherein each occurrence of cycloalkyl, aryl, heteroaryl, heterocyclyl, arylene, or heteroarylene is unsubstituted or substituted;

or a pharmaceutically acceptable salt thereof.

In an embodiment the compound has the structure

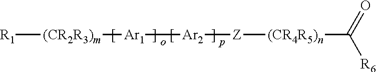

wherein

R₁ is H, halogen, —NR₇R₈, —NH—C(═O)—OR₇, —OR₇, —NO₂, —CN, —SR₇, —SO₂R₇, —CO₂R₇, CF₃, —SOR₇, —POR₇, —C(═S)R₇, —C(═O)—NR₇R₈, —C(═NR₇)R₈, —P(═O)(OR₇)(OR₈), —P(OR₇)(OR₈), —C(═S)R₇, C₁₋₅ alkyl, C₂₋₅ alkenyl, C₂₋₅ alkynyl, aryl, heteroaryl, or heterocyclyl,
wherein R₇ and R₈ are each, independently, H, C₁₋₅ alkyl, C₂₋₅ alkenyl, C₂₋₅alkynyl, aryl, or heteroaryl;

R₂ and R₃ are each, independently, H, —CH₂—R₉, —C(═O)OR₉, —C(═O)NR₉R₁₀, or —C(═O)R₉,
wherein
R₉ and R₁₀ are each, independently, H, C₁₋₁₀ alkyl, C₂₋₁₀ alkenyl, C₂₋₁₀ alkynyl, —(CH₂)ᵣ—OR₁₈,

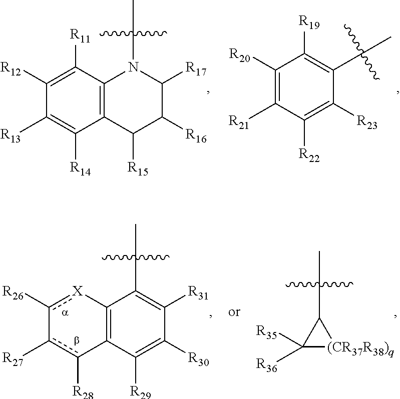

wherein
q is an integer from 1 to 6;
r is an integer from 1 to 10;
R₁₈ is H, C₁₋₁₀ alkyl, C₂₋₁₀ alkenyl, C₂₋₁₀ alkynyl, cycloalkyl, aryl, heteroaryl, or heterocyclyl;
bond α and bond β are each, independently, present or absent;
when bond α is present, X is N or CR₃₂;
when bond α is absent, X is NR₃₂ or CR₃₁R₃₂;
R₁₁, R₁₂, R₁₃, R₁₄, R₁₅, R₁₆, R₁₇, R₁₉, R₂₀, R₂₁, R₂₂, R₂₃, R₂₆, R₂₇, R₂₈, R₂₉, R₃₀, R₃₁, R₃₂, R₃₅, R₃, R₃₇, and R₃₈ are each, independently, H, halogen, —NO₂, —CN, —NR₂₄R₂₅, —SR₂₄, —SO₂R₂₄, —CO₂R₂₄, —OR₂₄, CF₃, —SOR₂₄, —POR₂₄, —C(═S)R₂₄, —C(═NR₂₄)R₂₅, —P(═O)(OR₂₄)(OR₂₅), —P(OR₂₄)(OR₂₅), —C(═S)R₂₄, C₁₋₁₀ alkyl, C₂₋₁₀ alkenyl, C₂₋₁₀ alkynyl, aryl, heteroaryl, or heterocyclyl;
   wherein R₂₄ and R₂₅ are each, independently, H, C₁₋₁₀ alkyl, C₂₋₁₀ alkenyl, C₂₋₁₀ alkynyl, aryl, heteroaryl, or heterocyclyl;
wherein R₂ or R₃ is other than H;
m is an integer from 0 to 5;

Ar₁ and Ar₂ are each, independently, arylene or a heteroarylene other than isooxazolylene;

o and p are each, independently, 0 or 1;

Z is —NR₄₃—C(═O)—, or a bond,
wherein R₄₃ is H, —(CR₄₄R₄₅)ₛ—CR₄₄R₄₅R₄₆, C₁₋₁₀ alkyl substituted by heterocyclyl, C₂₋₁₀ alkenyl, C₂₋₁₀ alkynyl, aryl, heteroaryl, or heterocyclyl,
   wherein
   s is an integer from 1 to 10;
   R₄₄, R₄₅, and R₄₆ are each, independently, H, halogen, —NO₂, —CN, —NR₄₇R₄₈, —SR₄₇, —SO₂R₄₇, —CO₂R₄₇, —OR₄₇, CF₃, —SOR₄₇, —POR₄₇, —C(—S)R₄₇, —C(═NR₄₇)R₄₈, —P(═O)(OR₄₇)(OR₄₈), —P(OR₄₇)(OR₄₈), —C(═S)R₄₇, C₁₋₁₀ alkyl, C₂₋₁₀ alkenyl, C₂₋₁₀ alkynyl, aryl, heteroaryl, or heterocyclyl,
      wherein R₄₇ and R₄₈ are each, independently, H, C₁₋₁₀ alkyl, C₂₋₁₀ alkenyl, C₂₋₁₀ alkynyl, aryl, heteroaryl, or heterocyclyl;
wherein when Z is a bond, o is 0, p is 0, R₂ or R₃ is C(═O)NR₉R₁₀, then R₉ and R₁₀ are each, independently, H, C₁₋₁₀ alkyl, C₂₋₁₀ alkenyl, C₂₋₁₀ alkynyl, —(CH₂)ᵣ—OR₁₈,

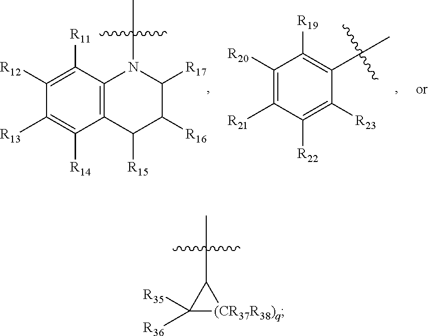

R₄ and R₅ are each, independently, H, C₁₋₁₀ alkyl, C₂₋₁₀ alkenyl, C₂₋₁₀ alkynyl, aryl, heteroaryl, or heterocyclyl;

n is an integer from 2 to 10;

R₆ is —OR₄₉ or —NH—OR₄₉,
wherein R₄₉ is H, C₁₋₁₀ alkyl, C₂₋₁₀ alkenyl, C₂₋₁₀ alkynyl, aryl, heteroaryl, or heterocyclyl;

wherein each occurrence of alkyl, alkenyl, or alkynyl is unsubstituted or substituted, branched or unbranched;

wherein each occurrence of cycloalkyl, aryl, heteroaryl, heterocyclyl, arylene, or heteroarylene is unsubstituted or substituted;

or a pharmaceutically acceptable salt thereof.

In an embodiment the compound has the structure

the structure

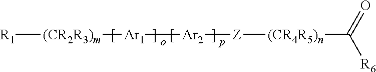

wherein

R₁ is H or —NH—C(═O)—OR₇,
wherein R₇ is C₁₋₅ alkyl;

R₂ and R₃ are each, independently, H, —CH₂—R₉, or —C(═O)NR₉R₁₀.
wherein
R₉ and R₁₀ are each, independently, H, C₁₋₁₀ alkyl, C₂₋₁₀ alkenyl, C₂₋₁₀ alkynyl, —

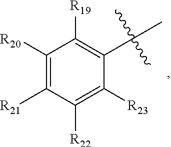

(CH₂)ᵣ—OR₁₈, or
   wherein
   r is an integer from 1 to 10;
   R₁₈ is H, C₁₋₁₀ alkyl, C₂₋₁₀ alkenyl, C₂₋₁₀ alkynyl, cycloalkyl, aryl, heteroaryl, or heterocyclyl;
   R₁₉, R₂₀, R₂₁, R₂₂, R₂₃ are each, independently, H, halogen, —NO₂, —CN, —NR₂₄R₂₅, —SR₂₄, —SO₂R₂₄, —CO₂R₂₄, —OR₂₄, CF₃, —SOR₂₄, —POR₂₄, —C(═S)R₂₄, —C(═NR₂₄)R₂₅, —P(═O)(OR₂₄)(OR₂₅), —P(OR₂₄)(OR₂₅), —C(—S)R₂₄, C₁₋₁₀ alkyl, C₂₋₁₀ alkenyl, C₂₋₁₀ alkynyl, aryl, heteroaryl, or heterocyclyl;
wherein R₂ or R₃ is other than H;

m is an integer from 0 to 5;

Ar₁ and Ar₂ are each, independently, arylene or a heteroarylene other than isooxazolylene;

o and p are each, independently, 0 or 1;

Z is —NH—C(—O)—, or a bond,
wherein when Z is a bond, o is 0, p is 0, R₂ or R₃ is C(═O)NR₉R₁₀, then
   R₉ and R₁₀ are each, independently, H, C₁₋₁₀ alkyl, C₂₋₁₀ alkenyl, C₂₋₁₀ alkynyl, —(CH₂)ᵣ—OR₁₈,

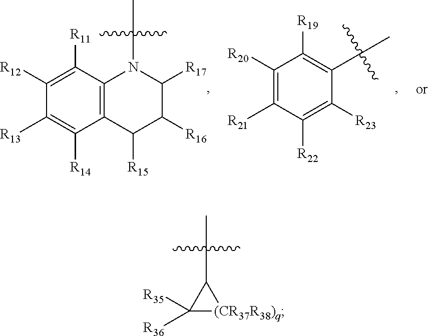

R₄ and R₅ are each H;

n is an integer from 2 to 10;

R₆ is —NH—OR₄₉,
wherein R₄₉ is H, C₁₋₁₀ alkyl, C₂₋₁₀ alkenyl, C₂₋₁₀ alkynyl, aryl, heteroaryl, or heterocyclyl;

wherein each occurrence of alkyl, alkenyl, or alkynyl is unsubstituted or substituted, branched or unbranched;

wherein each occurrence of cycloalkyl, aryl, heteroaryl, heterocyclyl, arylene, or heteroarylene is unsubstituted or substituted;

or a pharmaceutically acceptable salt thereof.

In an embodiment:

R₁ is H or —NH—C(═O)—O-tert-butyl;

R₂ and R₃ are each, independently, H, —CH₂—R₉, or —C(═O)NR₉R₁₀,
wherein
R₉ and R₁₀ are each, independently, H or

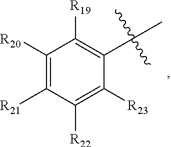

wherein R₁₉, R₂₀, R₂₁, R₂₂, R₂₃ are each, independently, H or tert-butyl;

wherein R₂ or R₃ is other than H;

m is an integer from 0 to 5;

Ar₁ and Ar₂ are each, independently, arylene or thiophenylene;

o and p are each, independently, 0 or 1;

Z is —NH—C(═O)—, or a bond,
wherein when Z is a bond, o is 0, p is 0, R₂ or R₃ is C(═O)NR₉R₁₀, then
   R₉ and R₁₀ are each, independently, H or

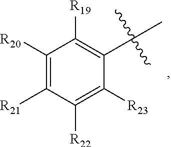

wherein R₁₉, R₂₀, R₂₁, R₂₂, R₂₃ are each, independently, H or tert-butyl;

R₄ and R₅ are each H;

n is an integer from 5 to 7;

R₆ is —NH—OH;

or a pharmaceutically acceptable salt thereof.

In an embodiment the compound has the structure:

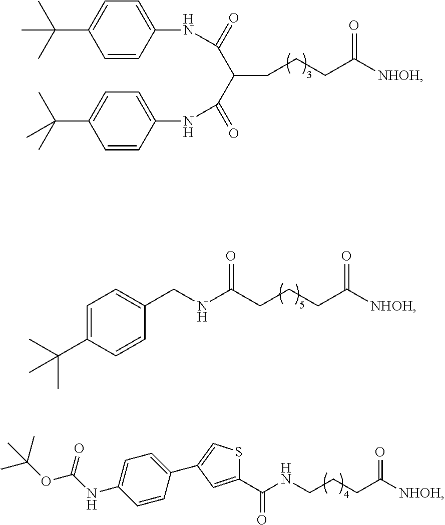

or a pharmaceutically acceptable salt thereof.

In an embodiment the compound has the structure:

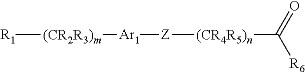

wherein

R₁ is H, halogen, —NR₇R₈, —NR₅₁—C(═O)—R₅₂, —OR₇, —NO₂, —CN, —SR₇, —SO₂R₇, —CO₂R₇, CF₃, —SOR₇, —POR₇, —C(═S)R₇, —C(═O)—NR₇R₈, —CH₂—C(═O)—NR₇R₈, —C(═NR₇)R₈, —P(═O)(OR₇)(OR₈), —P(OR₇)(OR), —C(═S)R₇, C₁₋₅ alkyl, C₂₋₅ alkenyl, C₂₋₅ alkynyl, aryl, heteroaryl, or heterocyclyl,
wherein R₇, R₈, R₅₁ and R₅₂ are each, independently, H, C₁₋₅ alkyl, C₂₋₅ alkenyl, C₂₋₅ alkynyl, aryl, or heteroaryl;
R₂ and R₃ are each, independently, H, —(NH₂), —C(═O)OR₉, —C(═O)NR₉R₁₀, or —C(═O)R₉,
   wherein
   R₉ and R₁₀ are each, independently, H, C₁₋₁₀ alkyl, C₂₋₁₀ alkenyl, C₂₋₁₀ alkynyl, —(CH₂)ᵣ—OR₁₈,

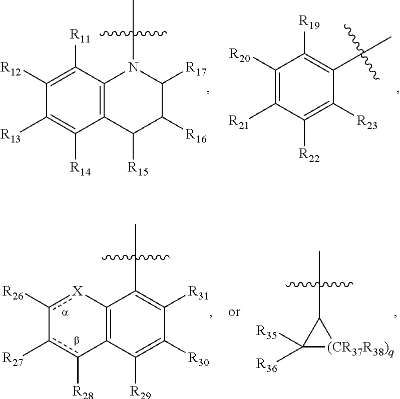

wherein
q is an integer from 1 to 6;
r is an integer from 1 to 10;
R₁₈ is H, C₁₋₁₀ alkyl, C₂₋₁₀ alkenyl, C₂₋₁₀ alkynyl, cycloalkyl, aryl, heteroaryl, or heterocyclyl;
bond α and bond β are each, independently, present or absent;
when bond α is present, X is N or CR₃₂;
when bond α is absent, X is NR₃₂ or CR₃₁R₃₂;
R₁₁, R₁₂, R₁₃, R₁₄, R₁₅, R₁₆, R₁₇, R₁₉, R₂₀, R₂₁, R₂₂, R₂₃ R₂₆, R₂₇, R₂₈, R₂₉, R₃₀, R₃₁, R₃₂, R₃₅, R₃₆, R₃₇, and R₃₈ are each, independently, H, halogen, —NO₂, —CN, —NR₂₄R₂₅, —SR₂₄, —SO₂R₂₄, —CO₂R₂₄, —OR₂₄, CF₃, —SOR₂₄, —POR₂₄, —C(═S)R₂₄, —C(═NR₂₄)R₂₅, —P(═O)(OR₂₄)(OR₂₅), —P(OR₂₄)(OR₂₅), —C(═S)R₂₄, C₁₋₁₀ alkyl, C₂₋₁₀ alkenyl, C₂₋₁₀ alkynyl, aryl, heteroaryl, or heterocyclyl;
   wherein R₂₄ and R₂ are each, independently, H, C₁₋₁₀ alkyl, C₂₋₁₀ alkenyl, C₂₋₁₀ alkynyl, aryl, heteroaryl, or heterocyclyl;
wherein R₂ or R₃ is other than H;

Ar₁ is arylene or heteroarylene, wherein the heteroarylene is not isooxazolylene;

m is an integer from 0 to 5;

Z is —NR₄₃—C(═O)—, or —C(═O)—NR₅₀—
wherein R₄₃ and R₅₀ are, independently, H, —(CR₄₄R₄₅)ₛ—CR₄₄R₄₅R₄₆, C₁₋₁₀ alkyl substituted by heterocyclyl, C₂₋₁₀ alkenyl, C₂₋₁₀ alkynyl, aryl, heteroaryl, or heterocyclyl,
   wherein
   s is an integer from 1 to 10;
   R₄₄, R₄₅, and R₄₆ are each, independently, H, halogen, —NO₂, —CN, —NR₄₇R₄₈, —SR₄₇, —SO₂R₄₇, —CO₂R₄₇, —OR₄₇, CF₃, —SOR₄₇, —POR₄₇, C(═S)R₄₇, —C(═NR₄₇)R₄, —P(═O)(OR₄₇)(OR₄₈), —P(OR₄₇)(OR₄₈), —C(═S)R₄₇, C₁₋₁₀ alkyl, C₂₋₁₀ alkenyl, C₂₋₁₀ alkynyl, aryl, heteroaryl, or heterocyclyl,
      wherein R₄₇ and R₄₈ are each, independently, H, C₁₋₁₀ alkyl, C₂₋₁₀ alkenyl, C₂₋₁₀ alkynyl, aryl, heteroaryl, or heterocyclyl;

R₄ and R₅ are each, independently, H, C₁₋₁₀ alkyl, C₂₋₁₀ alkenyl, C₂₋₁₀ alkynyl, aryl, heteroaryl, or heterocyclyl;

n is an integer and is 0 or from 2 to 10;

R₆ is —OR₄₉ or —NH—OR₄₉,
wherein R₄₉ is H, C₁₋₁₀ alkyl, C₂₋₁₀ alkenyl, C₂₋₁₀ alkynyl, aryl, heteroaryl, or heterocyclyl;

wherein each occurrence of alkyl, alkenyl, or alkynyl is unsubstituted or substituted, branched or unbranched;

wherein each occurrence of cycloalkyl, aryl, heteroaryl, heterocyclyl, arylene, or heteroarylene is unsubstituted or substituted;

or a pharmaceutically acceptable salt thereof.
In an embodiment, R₁ is H, F, —NH₂, —OH, —CH₃, —NR₅₁—C(═O)—R₅₂, —CH₂—C(═O)—NR₇R₈, or —(C═O)—NR₇R₈
   wherein R₇ is H, —C₂H₄OH, —CH₂—CHOH—CH₂OH, or aryl,
   wherein R₅₁ is H, —C₂H₄OH, or —CH₂—CHOH—CH₂OH,
   wherein R₈ and R₅₂ are, independently, a fluorine-substituted aryl, quinolinyl, or a
   nitrogen-containing heteroaryl having the structure:

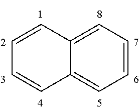

wherein the point of attachment is any one of atom positions 1, 2, 3, 4, 5, 6, 7, or 8, and wherein the nitrogen atom can be at any of atom positions 1, 2, 3, 4, 5, 6, 7, or 8, with the proviso that the point of attachment and the nitrogen atom are not at the same atom position,
or a heteroaryl comprising two nitrogen atoms having the structure:

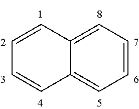

wherein the point of attachment is any one of atom positions 1, 2, 3, 4, 5, 6, 7, or 8, and wherein a first nitrogen atom can be at any of atom positions 1, 2, 3, 4, 5, 6, 7, or 8, and wherein a second nitrogen atom is at any of atom positions 1, 2, 3, 4, 5, 6, 7, or 8, with the provisos that (a) no nitrogen atom is directly bound to another nitrogen atom and (b) the point of attachment, and the first nitrogen atom, and the second nitrogen atom are each at different atom positions,
or a pharmaceutically acceptable salt thereof.

In an embodiment of the compound:
R₁ is H, halogen, —NR₇R₈, —OR₇, —(C═O)—NR₇R₈, or C₁₋₅ alkyl,
   wherein R₇ and R₈ are each, independently, H or heteroaryl;
or a pharmaceutically acceptable salt thereof.

In an embodiment of the compound:
Z is —C(═O)—NR₅₀—,
wherein R₅₀ is H or a C1-C5 alkyl,
or a pharmaceutically acceptable salt thereof.

In an embodiment of the compound:

R₁ is —NR₅₁—C(═O)—NR₅₂,
wherein R₅₁ is H or a C1-C5 alkyl and R₅₂ is heteroaryl,
or a pharmaceutically acceptable salt thereof.

In an embodiment of the compound:
R₂ and R₃ are each, independently, H, —C(═O)OR₉, —C(═O)NR₉R₁₀, or —C(═O)R₉,
   wherein
   R₉ and R₁₀ are each, independently, H, C₁₋₁₀ alkyl, —(CH₂)ᵣ—OR₁₈,

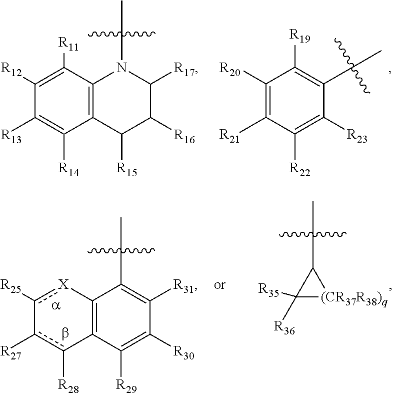

wherein
q is an integer from 1 to 6;
r is an integer from 1 to 10;
R₁₈ is H, C₁₋₁₀ alkyl, C₂₋₁₀ alkenyl, C₂₋₁₀ alkynyl, cycloalkyl, aryl, heteroaryl, or heterocyclyl;
bond α and bond β are each, independently, present or absent;
when bond α is present, X is N or CR₃₂;
when bond α is absent, X is NR₃₂ or CR₃₁R₃₂;
R₁₁, R₁₂, R₁₃, R₁₄, R₁₅, R₆, R₁₇, R₉, R₂₀, R₂₁, R₂₂, R₂₃ R₂₆, R₂₇, R₂₈, R₂₉, R₃₀, R₃₁, R₃₂, R₃₅, R₃₆, R₃₇, and R₃₈ are each, independently, H, halogen, —NO₂, —CN, —NR₂₄R₂₅, —SR₂₄, —SO₂R₂₄, —CO₂R₂₄, —OR₂₄, CF₃, —SOR₂₄, —POR₂₄, —C(═S)R₂₄, —C(═NR₂₄)R₂₅, —P(═O)(OR₂₄)(OR₂₅), —P(OR₂₄)(OR₂₅), —C(═S)R₂₄, C₁₋₁₀ alkyl, C₂₋₁₀ alkenyl, C₂₋₁₀ alkynyl, aryl, heteroaryl, or heterocyclyl;
wherein R₂₄ and R₂₅ are each, independently, H, C₁₋₁₀ alkyl, C₂₋₁₀ alkenyl, C₂₋₁₀ alkynyl, aryl, heteroaryl, or heterocyclyl;
wherein R₂ or R₃ is other than H;
or a pharmaceutically acceptable salt thereof.

In an embodiment of the compound:
Ar₁ is arylene;
or a pharmaceutically acceptable salt thereof.

In an embodiment the compound has the structure:

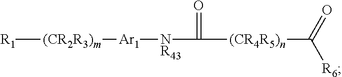

or a pharmaceutically acceptable salt thereof.

In an embodiment the compound has the structure:

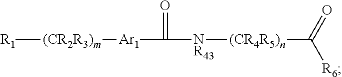

or a pharmaceutically acceptable salt thereof.

In an embodiment m is an integer from 0 to 2; or a pharmaceutically acceptable salt thereof.

In an embodiment n is an integer from 3 to 8; or a pharmaceutically acceptable salt thereof.

In an embodiment R₆ is —OR₄₉ or —NH—OR₄₉,
wherein R₄₉ is H or C₁₋₁₀ alkyl;
or a pharmaceutically acceptable salt thereof.

In an embodiment the compound has the structure

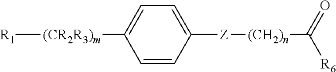

wherein
R₁ is H, F, —NH₂, —OH, —CH₃, or —(C═O)—NH—R₈,
   wherein R₈ is quinolinyl,
R₂ and R₃ are each, independently, H, —C(═O)OR₉, —C(═O)NR₉R₁₀, or —C(—O)R₉,
   wherein
   R₉ and R₁₀ are each, independently, H, tert-butyl, neopentyl, —(CH₂)₂—OH,

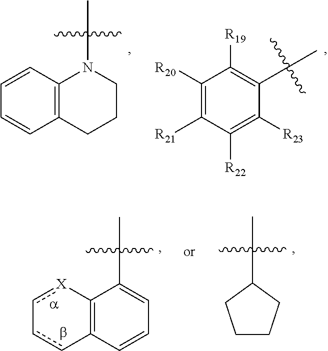

wherein R₁₉, R₂₀, R₂₁, R₂₂, R₂₃ are each, independently, H, tert-butyl, or F;
bond α and bond β are each, independently, present or absent;
when bond α is present, bond 1 is present and X is N or CH;
when bond α is absent, bond 1 is absent and X is CH₂;
wherein R₂ or R₃ is other than H;
m is 0 or 1;
Z is —NR₄₃—C(═O)—,
   wherein R₄₃ is H, —CH₂—CH(OH)—CH₂(OH), or

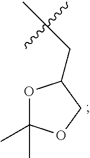 [SMILES: *CC1COC(C)(C)O1]

n is an integer from 5 to 7;
R₆ is —OR₄₉ or —NH—OR₄₉,
   wherein R₄₉ is H, —CH₃, —CH₂CH₃, or tert-butyl;
or a pharmaceutically acceptable salt thereof.

In an embodiment the compound has the structure

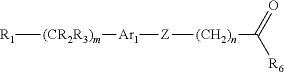

wherein
m is 0 or 1;
n is 0 or an integer from 5 to 7;
Ar₁ is

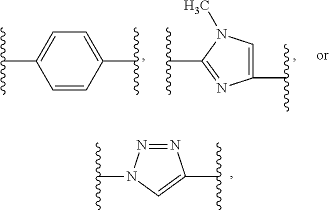

R₁ is H, F, —NH₂, —OH, —CH₃, —NR₅₁—C(═O)—R₅₂, —CH₂—C(═O)—NR₇R₈, or —(C═O)—NR₇R₈
   wherein R₇ is H, —C₂H₄OH, —CH₂—CHOH—CH₂OH, or aryl,
   wherein R₅₁ is H, —C₂H₄OH, or —CH₂—CHOH—CH₂OH,
   wherein R₈ and R₅₂ are, independently, a fluorine-substituted aryl, quinolinyl, or a nitrogen-containing heteroaryl having the structure:

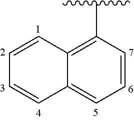 [SMILES: *c1cccc2ccccc12]

wherein the nitrogen atom can be at any of positions 2, 3, 4, 5, 6, or 7, or a heteroaryl comprising two nitrogen atoms having the structure:

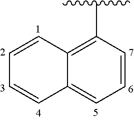 [SMILES: *c1cccc2ccccc12]

wherein one nitrogen atom is at position 1, 2, 3, 4, 5, 6, or 7 and the second nitrogen atom is in any one of the remaining numbered positions, with the proviso that no nitrogen atom is directly bound to another nitrogen atom,
R₂ and R₃ are, if present, each, independently, H, —(NH₂), —C(═O)OR₉, —C(═O)NR₉R₁₀, or —C(═O)R₉,
   wherein
   R₉ and R₁₀ are each, independently, H, tert-butyl, neopentyl, —(CH₂)₂—OH,

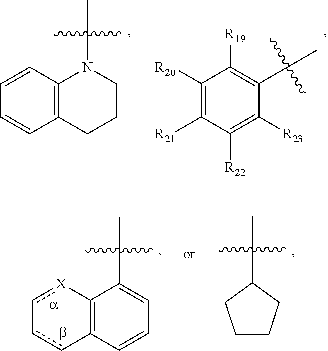

wherein R₁₉, R₂₀, R₂₁, R₂₂, R₂₃ are each, independently, H, tert-butyl, or F;
bond α and bond β are each, independently, present or absent;
when bond α is present, bond β is present and X is N or CH;
when bond α is absent, bond β is absent and X is CH₂;
wherein R₂ or R₃ is other than H;
wherein Z is —NR₄₃—C(═O)—, or is —C(═O)—NR₅₀—, wherein R₅₀ is H,
   wherein R₄₃ is H, —CH H(OH)—CH₂(OH), or

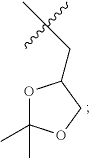

wherein R₆ is —OR₄₉ or —NH—OR₄₉,
   wherein R₄₉ is H, —CH₃, —CH₂CH₃, or tert-butyl;
or a pharmaceutically acceptable salt thereof.

In an embodiment R₆ is —OR₄₉, —OH, or —NH—OR₄₉,
wherein R₄₉ is —CH₃, —CH₂CH₃, or tert-butyl;
or a pharmaceutically acceptable salt thereof.

In an embodiment R₄ is —NH—OH,
or a pharmaceutically acceptable salt thereof.
In an embodiment R₁ is —CH₂—C(═O)—NR₇R₈ or —(C═O)—NR₇R₈, wherein R₇ is H, —C₂H₄OH, —CH₂—CHOH—CH₂OH, or aryl and R₈ is aryl;
   Ar₁ is arylene;
   m is an integer from 0 to 5;
   Z is —NR₄₃—C(═O)—, wherein R₄₃ is H or C₁₋₄ alkyl,
   or a pharmaceutically acceptable salt thereof.

In an embodiment R₁ is —CH₂—C(═O)—NR₇R₈ wherein R₇ is —C₂H₄OH or aryl and R₈ is phenyl
or naphthalenyl;
R₄ and R₅ are both H;
R₆ is —NH—OH;
Ar₁ is arylene;
m is 0;
n is 6;
Z is —NH—C(═O)—,

or a pharmaceutically acceptable salt thereof.

In an embodiment the compound has the structure:

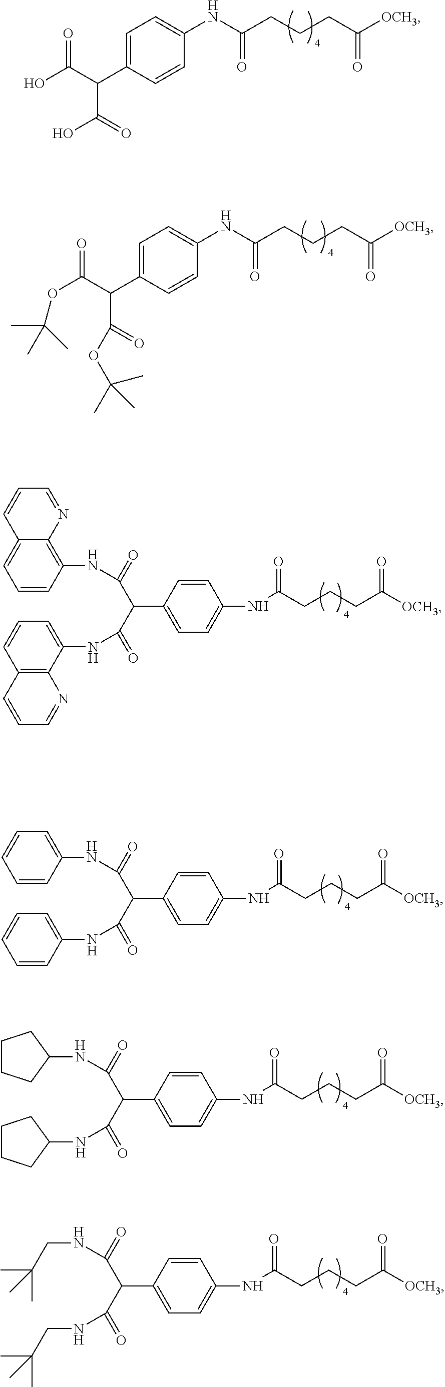

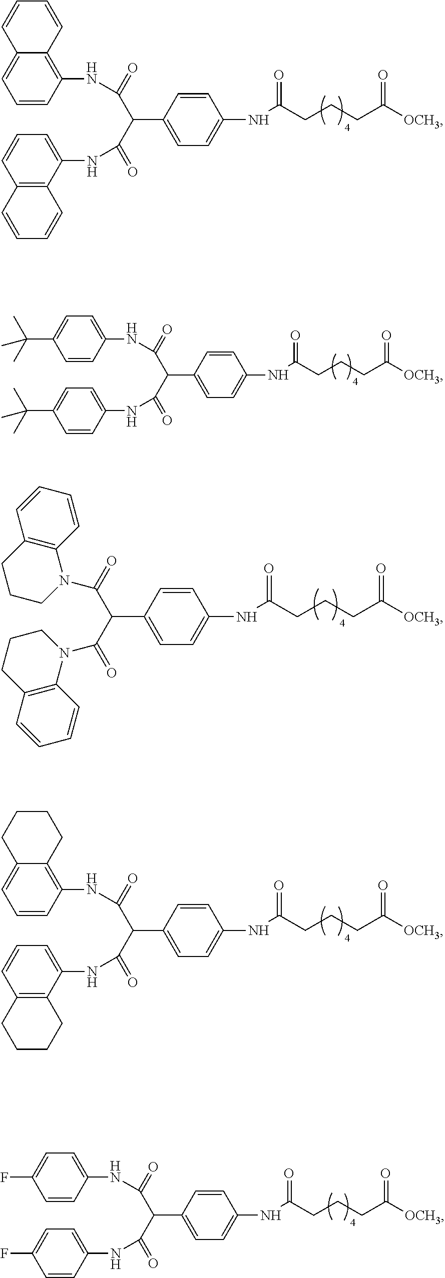

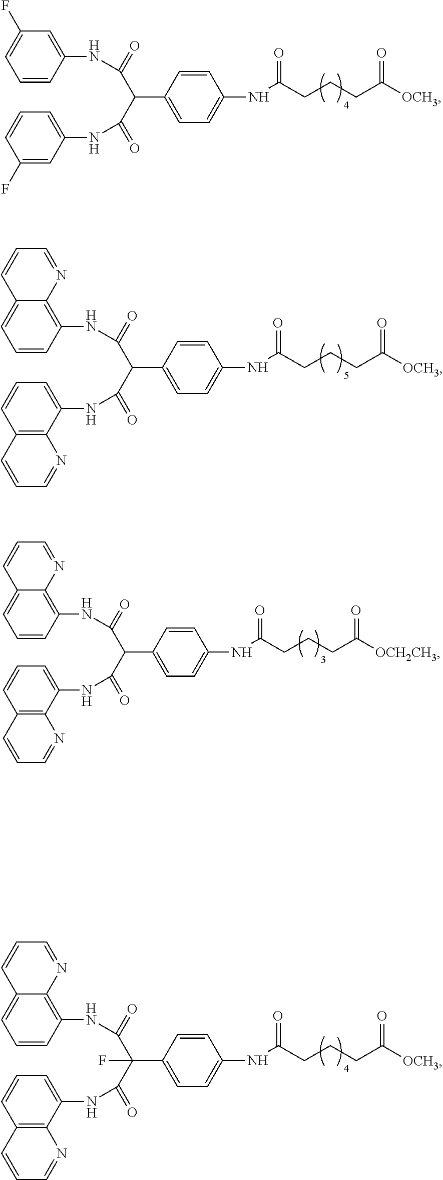

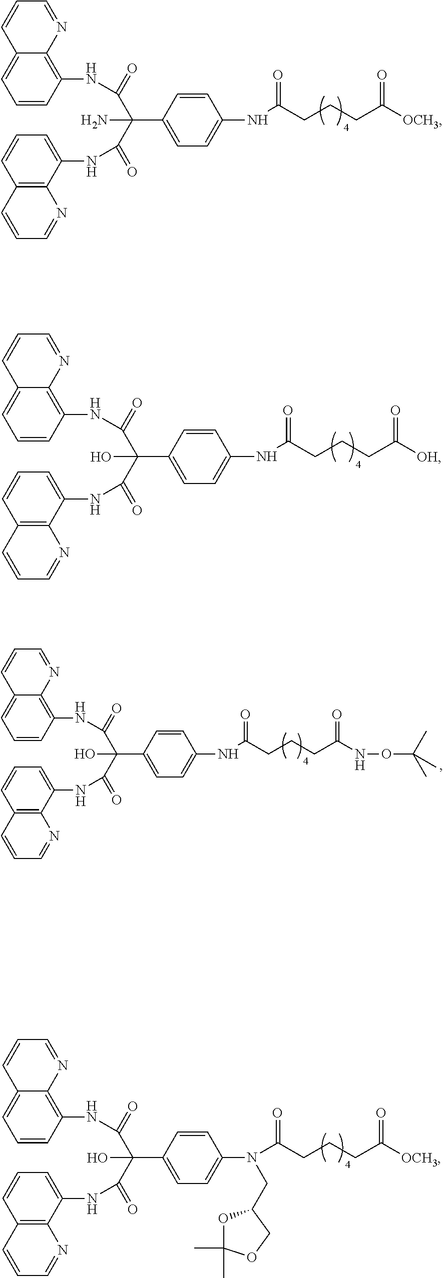

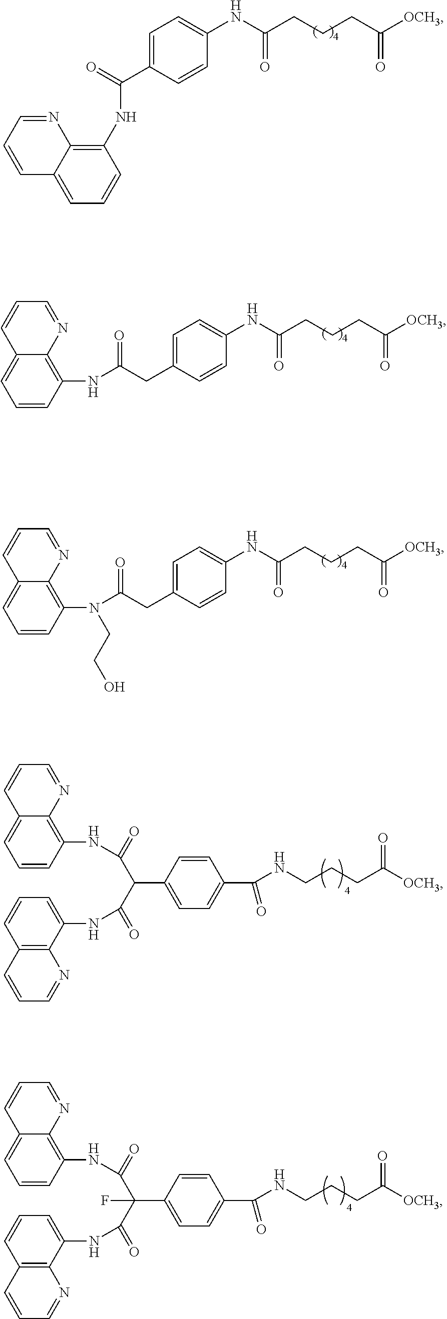

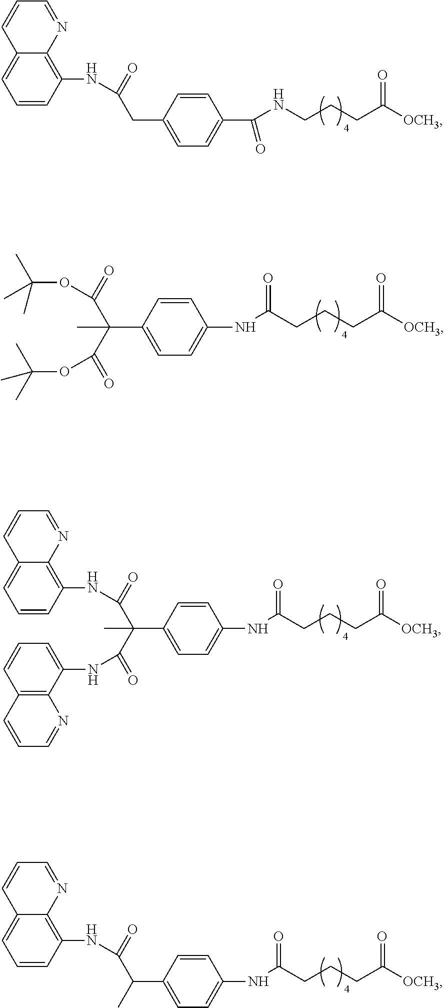

or a pharmaceutically acceptable salt thereof.

In an embodiment the compound has the structure:

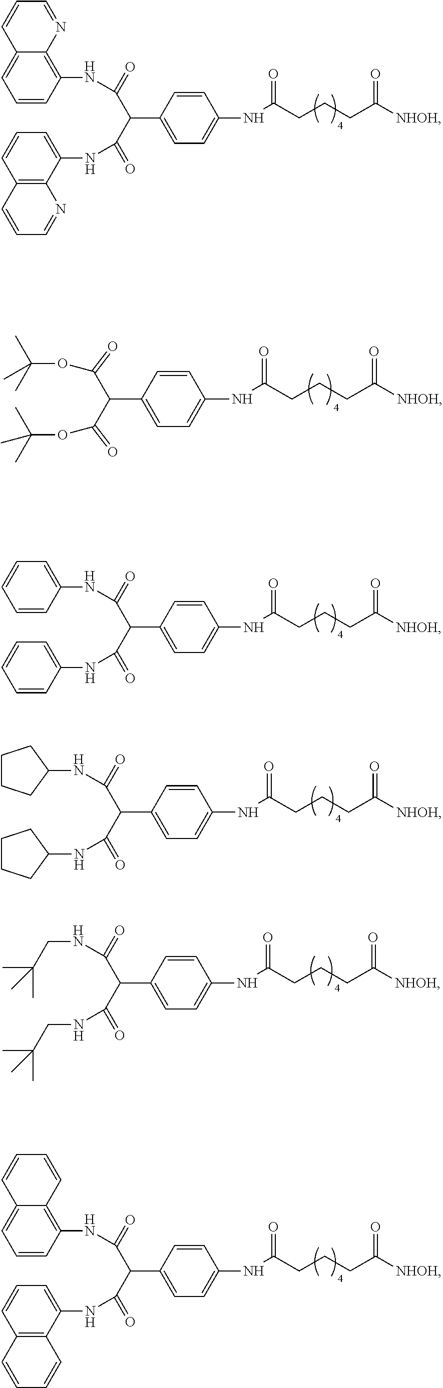

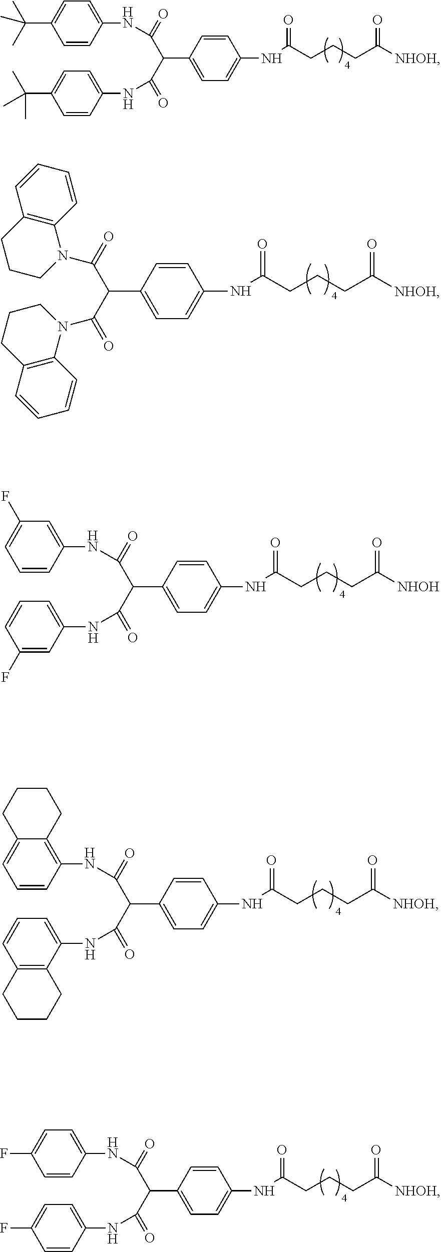

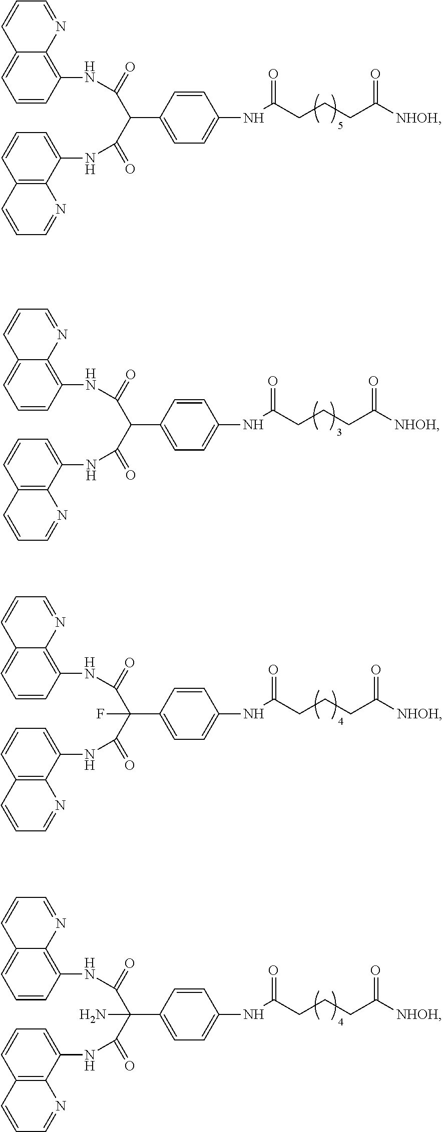

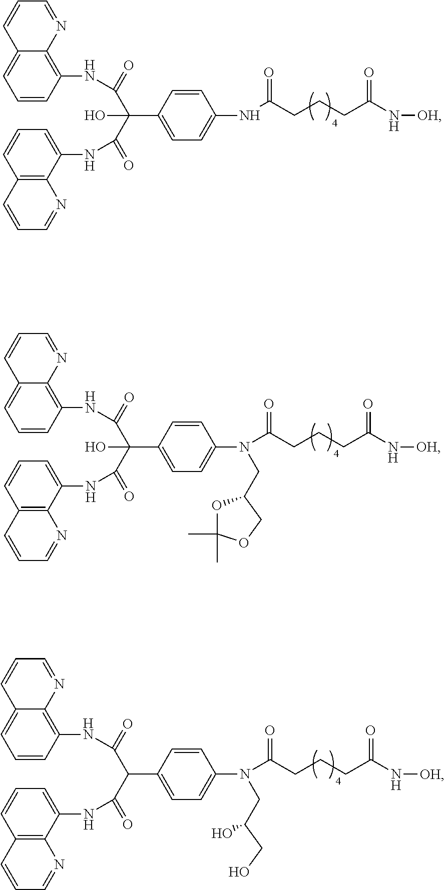

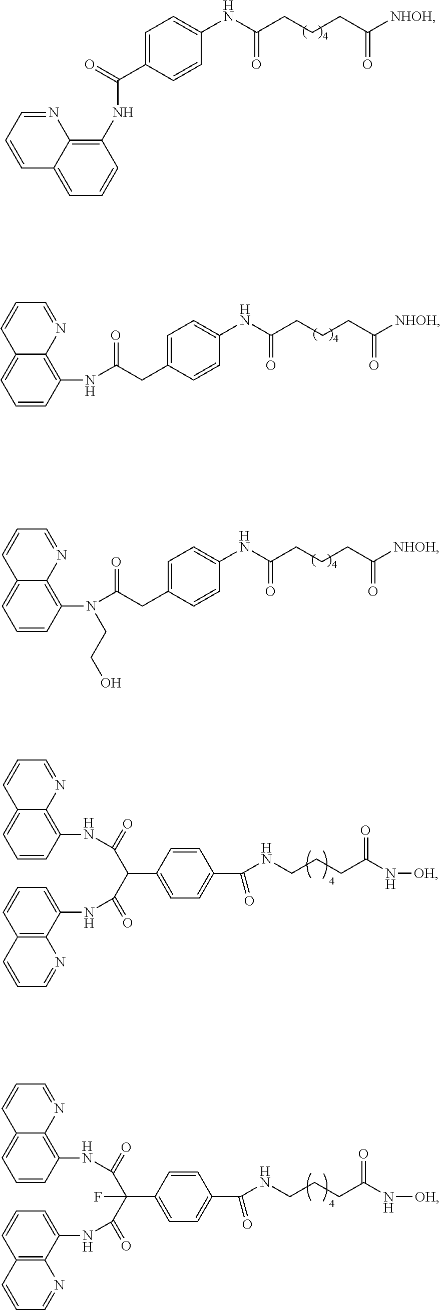

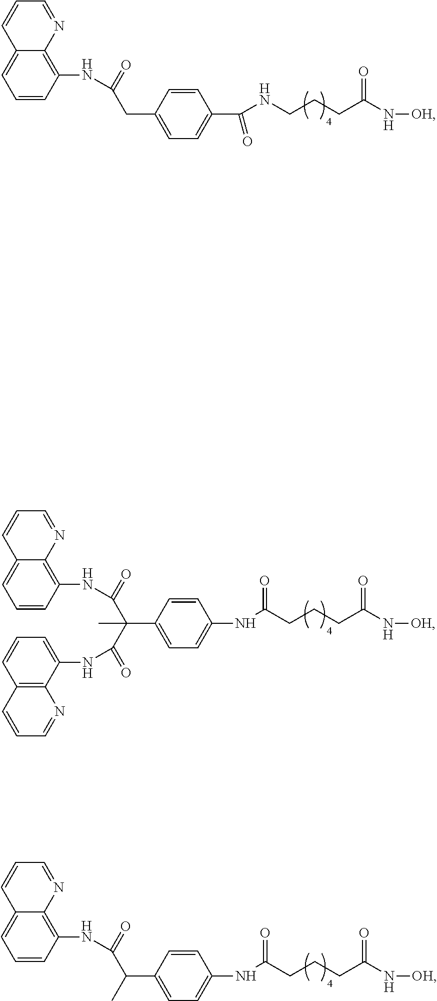

or a pharmaceutically acceptable salt thereof.

In an embodiment the compound has the structure:

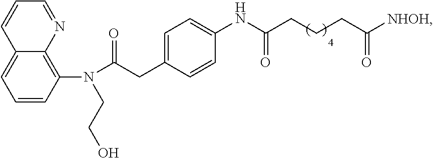

or a pharmaceutically acceptable salt thereof.

In an embodiment the compound has the structure:

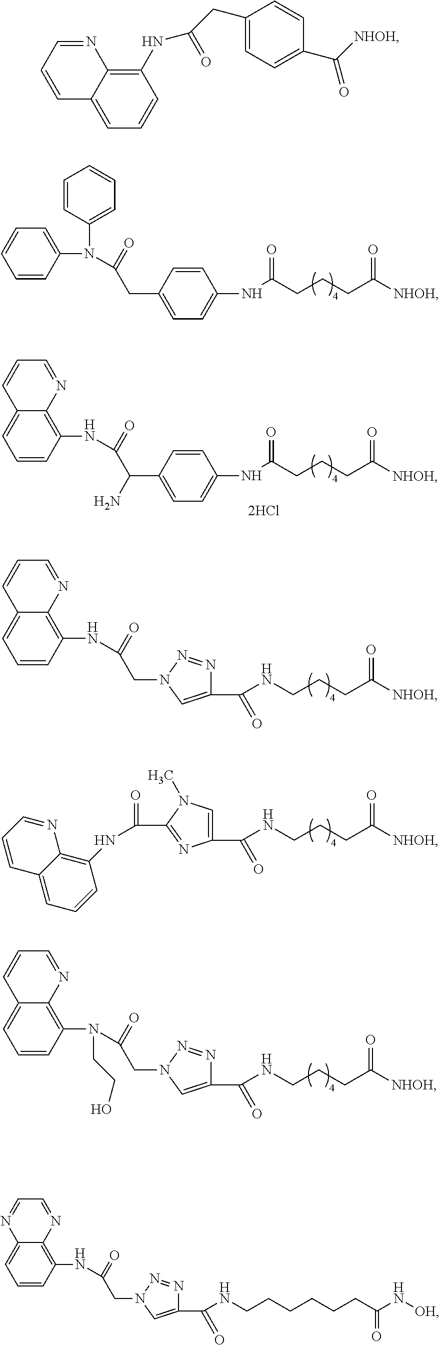

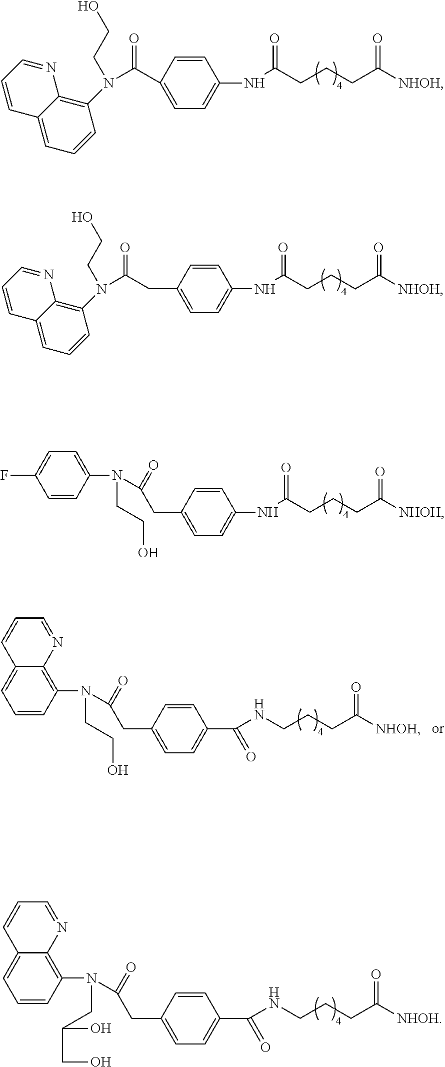

In an embodiment the compound has the structure:

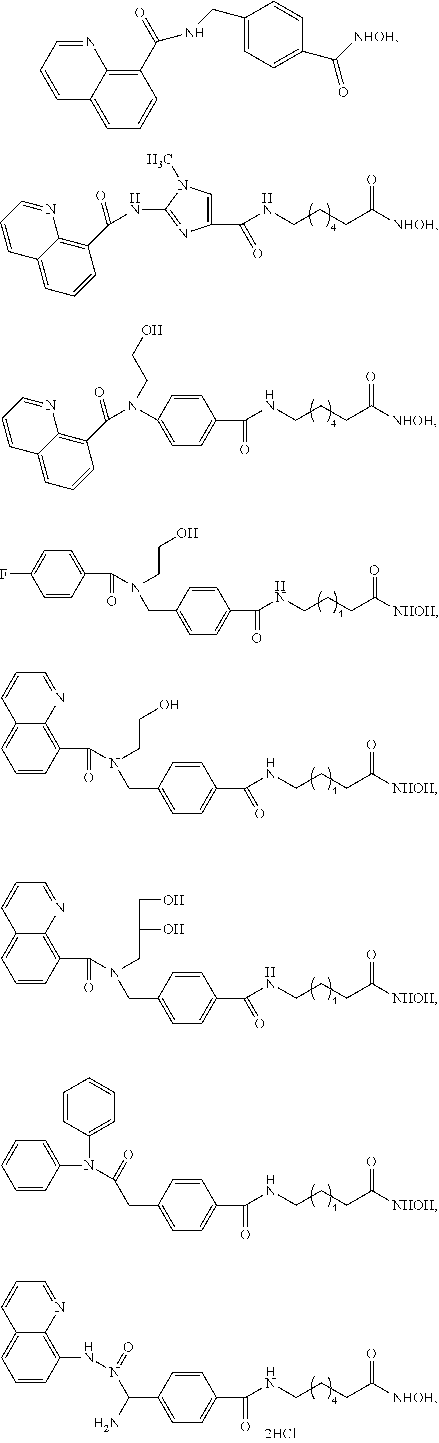

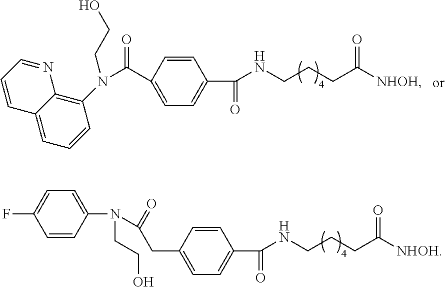

In an embodiment the compound has the structure:

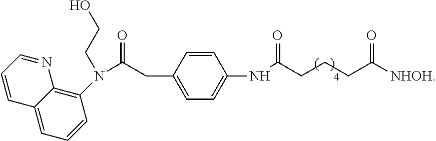

In an embodiment the compound has the structure:

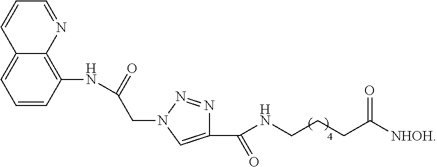

In an embodiment the compound has the structure:

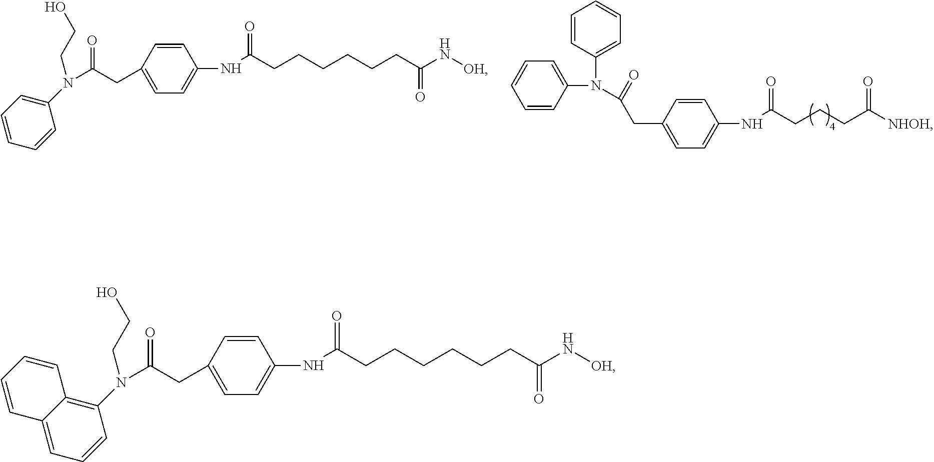

or a pharmaceutically acceptable salt thereof.

A pharmaceutical composition comprising any one, or more, of the instant compounds and a pharmaceutically acceptable carrier.

A method of inhibiting the activity of a histone deactylase in a cell comprising contacting the histone deacetylase with any one, or more, of the instant compounds so as to inhibit the activity of the histone deacetylase.

In an embodiment the histone deacetylase is HDAC6.

A method of inhibiting the activity of a histone deacetylase 6 (HDAC6) in a cell comprising contacting the histone deacetylase 6 with any one, or more, of the instant compounds so as to inhibit the activity of the histone deacetylase 6 in the cell.

A method of increasing accumulation of acetylated alpha tubulin in a cell comprising contacting the cell with any one, or more, of the instant compounds so as to increase the accumulation of acetylated alpha-tubulin in the cell.

This invention also provides isotopic variants of the compounds disclosed herein, including wherein the isotopic atom is ²H and/or wherein the isotopic atom ¹³C. Accordingly, in the compounds provided herein hydrogen can be enriched in the deuterium isotope. It is to be understood that the invention encompasses all such isotopic forms which inhibit HDAC, including those which inhibit HDAC6 selectively over HDAC1.

In an embodiment, the histone deacetylase is HDAC6.

A method of treating a neurodegenerative disease in a subject comprising administering an effective amount of any one, or more, of the instant compounds to the subject so as to treat the neurodegenerative disease in the subject.

In an embodiment, the neurodegenerative disease is Parkinson's disease, Alzheimer's disease, and Huntington's disease or Niemann-Pick type C disease.

A method of treating a disease associated with defective lipid transport in a subject comprising administering an effective amount of any one, or more, of the instant compounds to the subject so as to treat the disease in the subject.

In an embodiment, the disease associated with defective lipid transport is Stargardt macular degeneration, Harlequin ichthyosis or Tangier disease.

It is understood that the structures described in the embodiments of the methods hereinabove can be the same as the structures of the compounds described hereinabove.

It is understood that where a numerical range is recited herein, the present invention contemplates each integer between, and including, the upper and lower limits, unless otherwise stated.

As used herein, the term “activity” refers to the activation, production, expression, synthesis, intercellular effect, and/or pathological or aberrant effect of the referenced molecule, either inside and/or outside of a cell. Such molecules include, but are not limited to, cytokines, enzymes, growth factors, pro-growth factors, active growth factors, and pro-enzymes. Molecules such as cytokines, enzymes, growth factors, pro-growth factors, active growth factors, and pro-enzymes may be produced, expressed, or synthesized within a cell where they may exert an effect. Such molecules may also be transported outside of the cell to the extracellular matrix where they may induce an effect on the extracellular matrix or on a neighboring cell. It is understood that activation of inactive cytokines, enzymes and pro-enzymes may occur inside and/or outside of a cell and that both inactive and active forms may be present at any point inside and/or outside of a cell. It is also understood that cells may possess basal levels of such molecules for normal function and that abnormally high or low levels of such active molecules may lead to pathological or aberrant effects that may be corrected by pharmacological intervention.

As used herein, the term “histone deacetylase” or “HDAC” refers to any member of the classes of enzymes capable of cleaving an acetyl group (—C(═O)CH₃) from proteins, which include, but are not limited to, histones and microtubules. A histone deacetylase may be zinc-dependent. Examples of HDACs include, but are not limited to, HDAC1, HDAC2, HDAC3, HDAC4, HDAC5, HDAC6, HDAC7, HDAC8, HDAC9, HDAC10, and HDAC11.

The compounds of the present invention include all hydrates, solvates, and complexes of the compounds used by this invention. If a chiral center or another form of an isomeric center is present in a compound of the present invention, all forms of such isomer or isomers, including enantiomers and diastereomers, are intended to be covered herein. Compounds containing a chiral center may be used as a racemic mixture, an enantiomerically enriched mixture, or the racemic mixture may be separated using well-known techniques and an individual enantiomer may be used alone. The compounds described in the present invention are in racemic form or as individual enantiomers. The enantiomers can be separated using known techniques, such as those described in Pure and Applied Chemistry 69, 1469-1474, (1997) IUPAC. In cases in which compounds have unsaturated carbon-carbon double bonds, both the cis (Z) and trans (E) isomers are within the scope of this invention.

The compounds of the subject invention may have spontaneous tautomeric forms. In cases wherein compounds may exist in tautomeric forms, such as keto-enol tautomers, each tautomeric form is contemplated as being included within this invention whether existing in equilibrium or predominantly in one form.

In the compound structures depicted herein, hydrogen atoms are not shown for carbon atoms having less than four bonds to non-hydrogen atoms. However, it is understood that enough hydrogen atoms exist on said carbon atoms to satisfy the octet rule.

As used herein, “alkyl” includes both branched and straight-chain saturated aliphatic hydrocarbon groups having the specified number of carbon atoms and may be unsubstituted or substituted. Thus, C₁-Cₙ as in “C₁-Cₙ alkyl” is defined to include groups having 1, 2 . . . , n−1 or n carbons in a linear or branched arrangement. For example, C₁-C₆, as in “C₁-C₆ alkyl” is defined to include groups having 1, 2, 3, 4, 5, or 6 carbons in a linear or branched arrangement, and specifically includes methyl, ethyl, n-propyl, isopropyl, n-butyl, t-butyl, pentyl, hexyl, and octyl.

As used herein, “alkenyl” refers to a non-aromatic hydrocarbon radical, straight or branched, containing at least 1 carbon to carbon double bond, and up to the maximum possible number of non-aromatic carbon-carbon double bonds may be present, and may be unsubstituted or substituted. For example, “C₂-C₆ alkenyl” means an alkenyl radical having 2, 3, 4, 5, or 6 carbon atoms, and up to 1, 2, 3, 4, or 5 carbon-carbon double bonds respectively. Alkenyl groups include ethenyl, propenyl, butenyl and cyclohexenyl.

The term “alkynyl” refers to a hydrocarbon radical straight or branched, containing at least 1 carbon to carbon triple bond, and up to the maximum possible number of non-aromatic carbon-carbon triple bonds may be present, and may be unsubstituted or substituted. Thus, “C₂-C₆ alkynyl” means an alkynyl radical having 2 or 3 carbon atoms and 1 carbon-carbon triple bond, or having 4 or 5 carbon atoms and up to 2 carbon-carbon triple bonds, or having 6 carbon atoms and up to 3 carbon-carbon triple bonds. Alkynyl groups include ethynyl, propynyl and butynyl.

“Alkylene”, “alkenylene” and “alkynylene” shall mean, respectively, a divalent alkane, alkene and alkyne radical, respectively. It is understood that an alkylene, alkenylene, and alkynylene may be straight or branched. An alkylene, alkenylene, and alkynylene may be unsubstituted or substituted.

As used herein, “aryl” is intended to mean any stable monocyclic, bicyclic or polycyclic carbon ring of up to 10 atoms in each ring, wherein at least one ring is aromatic, and may be unsubstituted or substituted. Examples of such aryl elements include phenyl, p-toluenyl (4-methylphenyl), naphthyl, tetrahydro-naphthyl, indanyl, biphenyl, phenanthryl, anthryl or acenaphthyl. In cases where the aryl substituent is bicyclic and one ring is non-aromatic, it is understood that attachment is via the aromatic ring.

As used herein, the term “polycyclic” refers to unsaturated or partially unsaturated multiple fused ring structures, which may be unsubstituted or substituted.

The term “arylalkyl” refers to alkyl groups as described above wherein one or more bonds to hydrogen contained therein are replaced by a bond to an aryl group as described above. It is understood that an “arylalkyl” group is connected to a core molecule through a bond from the alkyl group and that the aryl group acts as a substituent on the alkyl group. Examples of arylalkyl moieties include, but are not limited to, benzyl(phenylmethyl), p-trifluoromethylbenzyl (4-trifluoromethylphenylmethyl), 1-phenylethyl, 2-phenylethyl, 3-phenylpropyl, 2-phenylpropyl and the like.

The term “heteroaryl”, as used herein, represents a stable monocyclic, bicyclic or polycyclic ring of up to 10 atoms in each ring, wherein at least one ring is aromatic and contains from 1 to 4 heteroatoms selected from the group consisting of O, N and S. Bicyclic aromatic heteroaryl groups include phenyl, pyridine, pyrimidine or pyridizine rings that are (a) fused to a 6-membered aromatic (unsaturated) heterocyclic ring having one nitrogen atom; (b) fused to a 5- or 6-membered aromatic (unsaturated) heterocyclic ring having two nitrogen atoms; (c) fused to a 5-membered aromatic (unsaturated) heterocyclic ring having one nitrogen atom together with either one oxygen or one sulfur atom; or (d) fused to a 5-membered aromatic (unsaturated) heterocyclic ring having one heteroatom selected from O, N or S. Heteroaryl groups within the scope of this definition include but are not limited to: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrazolyl, tetrazolopyridyl, thiadiazolyl, thiazolyl, thienyl, triazolyl, azetidinyl, aziridinyl, 1,4-dioxanyl, hexahydroazepinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, tetrahydrothienyl, acridinyl, carbazolyl, cinnolinyl, quinoxalinyl, pyrrazolyl, indolyl, benzotriazolyl, benzothiazolyl, benzoxazolyl, isoxazolyl, isothiazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetra-hydroquinoline. In cases where the heteroaryl substituent is bicyclic and one ring is non-aromatic or contains no heteroatoms, it is understood that attachment is via the aromatic ring or via the heteroatom containing ring, respectively. If the heteroaryl contains nitrogen atoms, it is understood that the corresponding N-oxides thereof are also encompassed by this definition.

The term “heterocycle”, “heterocyclyl” or “heterocyclic” refers to a mono- or poly-cyclic ring system which can be saturated or contains one or more degrees of unsaturation and contains one or more heteroatoms. Preferred heteroatoms include N, O, and/or S, including N-oxides, sulfur oxides, and dioxides. Preferably the ring is three to ten-membered and is either saturated or has one or more degrees of unsaturation. The heterocycle may be unsubstituted or substituted, with multiple degrees of substitution being allowed. Such rings may be optionally fused to one or more of another “heterocyclic” ring(s), heteroaryl ring(s), aryl ring(s), or cycloalkyl ring(s). Examples of heterocycles include, but are not limited to, tetrahydrofuran, pyran, 1,4-dioxane, 1,3-dioxane, piperidine, piperazine, pyrrolidine, morpholine, thiomorpholine, tetrahydrothiopyran, tetrahydrothiophene, 1,3-oxathiolane, and the like.

The alkyl, alkenyl, alkynyl, aryl, heteroaryl and heterocyclyl substituents may be substituted or unsubstituted, unless specifically defined otherwise.

In the compounds of the present invention, alkyl, alkenyl, alkynyl, aryl, heterocyclyl and heteroaryl groups can be further substituted by replacing one or more hydrogen atoms with alternative non-hydrogen groups. These include, but are not limited to, halo, hydroxy, mercapto, amino, carboxy, cyano and carbamoyl.

As used herein, the term “halogen” refers to F, Cl, Br, and I.

The term “substituted” refers to a functional group as described above in which one or more bonds to a hydrogen atom contained therein are replaced by a bond to non-hydrogen or non-carbon atoms, provided that normal valencies are maintained and that the substitution results in a stable compound. Substituted groups also include groups in which one or more bonds to a carbon(s) or hydrogen(s) atom are replaced by one or more bonds, including double or triple bonds, to a heteroatom. Examples of substituents include the functional groups described above, and, in particular, halogens (i.e., F, Cl, Br, and I); alkyl groups, such as methyl, ethyl, n-propyl, isopropryl, n-butyl, tert-butyl, neopentyl, and trifluoromethyl; hydroxyl; alkoxy groups, such as methoxy, ethoxy, n-propoxy, and isopropoxy; aryloxy groups, such as phenoxy; arylalkyloxy, such as benzyloxy(phenylmethoxy) and p-trifluoromethylbenzyloxy(4-trifluoromethylphenylmethoxy); heteroaryloxy groups; sulfonyl groups, such as trifluoromethanesulfonyl, methanesulfonyl, and p-toluenesulfonyl; nitro, nitrosyl; mercapto; sulfanyl groups, such as methylsulfanyl, ethylsulfanyl and propylsulfanyl; cyano; amino groups, such as amino, methylamino, dimethylamino, ethylamino, and diethylamino; and carboxyl. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

In choosing the compounds of the present invention, one of ordinary skill in the art will recognize that the various substituents, i.e. R₁, R₂, etc. are to be chosen in conformity with well-known principles of chemical structure connectivity.

The various R groups attached to the aromatic rings of the compounds disclosed herein may be added to the rings by standard procedures, for example those set forth in Advanced Organic Chemistry Part B: Reaction and Synthesis, Francis Carey and Richard Sundberg, (Springer) 5th ed. Edition. (2007), the content of which is hereby incorporated by reference.

The compounds of the instant invention may be in a salt form. As used herein, a “salt” is the salt of the instant compounds which has been modified by making acid or base salts of the compounds. Acidic substances can form salts with acceptable bases, including, but not limited to, lysine, arginine, and the like. In the case of compounds administered to a subject, eg. a human, the salt is pharmaceutically acceptable. Examples of pharmaceutically acceptable salts include, but are not limited to, mineral or organic acid salts formed at basic residues such as amino groups; alkali or organic base salts formed at acidic residues such as phenols, carboxylic acids, and carbons having at least 1 acidic hydrogen atom adjacent to a carbonyl. Where acid salts are formed, such salts can be made using an organic or inorganic acid. Such acid salts include, but are not limited to, chlorides, bromides, sulfates, nitrates, phosphates, sulfonates, formates, tartrates, maleates, malates, citrates, benzoates, salicylates, ascorbates, and the like. Because the compounds of the subject invention also possess carbons having at least 1 acidic hydrogen atom adjacent to a carbonyl, enolate salts may be formed by reaction with a suitable base. Suitable bases include, but are not limited, to inorganic bases, such as alkali and alkaline earth metal hydroxides; and organic bases, including, but not limited to, ammonia, alkyl amines, amino alcohols, amino sugars, amino acids, such as glycine, histidine, and lysine, and alkali metal amides, such as lithium diisopropylamide. The term “pharmaceutically acceptable salt” in this respect, refers to the relatively non-toxic, inorganic and organic acid or base addition salts of compounds of the present invention. These salts can be prepared in situ during the final isolation and purification of the compounds of the invention, or by separately reacting a purified compound of the invention in its free base or free acid form with a suitable organic or inorganic acid or base, and isolating the salt thus formed. Representative salts include the hydrobromide, hydrochloride, sulfate, bisulfate, phosphate, nitrate, acetate, valerate, oleate, palmitate, stearate, laurat, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci. 66:1-19).

The compounds and compositions of this invention may be administered in various forms, including those detailed herein. The treatment with the compound may be a component of a combination therapy or an adjunct therapy, i.e. the subject or patient in need of the drug is treated or given another drug for the disease in conjunction with one or more of the instant compounds. This combination therapy can be sequential therapy where the patient is treated first with one drug and then the other or the two drugs are given simultaneously. These can be administered independently by the same route or by two or more different routes of administration depending on the dosage forms employed.

As used herein, a “pharmaceutically acceptable carrier” is a pharmaceutically acceptable solvent, suspending agent or vehicle, for delivering the instant compounds to the animal or human. The carrier may be liquid or solid and is selected with the planned manner of administration in mind. Liposomes are also a pharmaceutically acceptable carrier.

The dosage of the compounds administered in treatment will vary depending upon factors such as the pharmacodynamic characteristics of a specific chemotherapeutic agent and its mode and route of administration; the age, sex, metabolic rate, absorptive efficiency, health and weight of the recipient; the nature and extent of the symptoms; the kind of concurrent treatment being administered; the frequency of treatment with; and the desired therapeutic effect.

The compounds and compositions of the present invention can be administered in oral dosage forms as tablets, capsules, pills, powders, granules, elixirs, tinctures, suspensions, syrups, and emulsions. The compounds may also be administered in intravenous (bolus or infusion), intraperitoneal, subcutaneous, or intramuscular form, or introduced directly, e.g. by topical administration, injection or other methods, to the afflicted area, such as a wound, including ulcers of the skin, all using dosage forms well known to those of ordinary skill in the pharmaceutical arts.

The compounds can be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit will be in a form suitable for oral, rectal, topical, intravenous or direct injection or parenteral administration. The compounds can be administered alone but are generally mixed with a pharmaceutically acceptable carrier. This carrier can be a solid or liquid, and the type of carrier is generally chosen based on the type of administration being used. In one embodiment the carrier can be a monoclonal antibody. The active agent can be co-administered in the form of a tablet or capsule, liposome, as an agglomerated powder or in a liquid form. Examples of suitable solid carriers include lactose, sucrose, gelatin and agar. Capsule or tablets can be easily formulated and can be made easy to swallow or chew; other solid forms include granules, and bulk powders. Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules.

Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms may also include minerals and other materials to make them compatible with the type of injection or delivery system chosen.

Specific examples of pharmaceutical acceptable carriers and excipients that may be used to formulate oral dosage forms of the present invention are described in U.S. Pat. No. 3,903,297 to Robert, issued Sep. 2, 1975. Techniques and compositions for making dosage forms useful in the present invention are described-in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modem Pharmaceutics Drugs and the Pharmaceutical Sciences, Vol 40 (Gilbert S. Banker, Christopher T. Rhodes, Eds.). All of the aforementioned publications are incorporated by reference herein.

Tablets may contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol and the like. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

The compounds can also be administered in the form of liposome delivery systems, such as small unilamellar vesicles, large unilamallar vesicles, and multilamellar vesicles. Liposomes can be formed from a variety of phospholipids, such as cholesterol, stearylamine, or phosphatidylcholines. The compounds may be administered as components of tissue-targeted emulsions.

The compounds may also be coupled to soluble polymers as targetable drug carriers or as a prodrug. Such polymers include polyvinylpyrrolidone, pyran copolymer, polyhydroxylpropylmethacrylamide-phenol, polyhydroxyethylasparta-midephenol, or polyethyleneoxide-polylysine substituted with palmitoyl residues. Furthermore, the compounds may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

The term “prodrug” as used herein refers to any compound that when administered to a biological system generates the compound of the invention, as a result of spontaneous chemical reaction(s), enzyme catalyzed chemical reaction(s), photolysis, and/or metabolic chemical reaction(s). A prodrug is thus a covalently modified analog or latent form of a compound of the invention.

The active ingredient can be administered orally in solid dosage forms, such as capsules, tablets, powders, and chewing gum; or in liquid dosage forms, such as elixirs, syrups, and suspensions, including, but not limited to, mouthwash and toothpaste. It can also be administered parentally, in sterile liquid dosage forms.

Solid dosage forms, such as capsules and tablets, may be enteric coated to prevent release of the active ingredient compounds before they reach the small intestine. Materials that may be used as enteric coatings include, but are not limited to, sugars, fatty acids, waxes, shellac, cellulose acetate phthalate (CAP), methyl acrylate-methacrylic acid copolymers, cellulose acetate succinate, hydroxy propyl methyl cellulose phthalate, hydroxy propyl methyl cellulose acetate succinate (hypromellose acetate succinate), polyvinyl acetate phthalate (PVAP), and methyl methacrylate-methacrylic acid copolymers.

Gelatin capsules may contain the active ingredient compounds and powdered carriers, such as lactose, starch, cellulose derivatives, magnesium stearate, stearic acid, and the like. Similar diluents can be used to make compressed tablets. Both tablets and capsules can be manufactured as immediate release products or as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

For oral administration in liquid dosage form, the oral drug components are combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents.

Liquid dosage forms for oral administration can contain coloring and flavoring to increase patient acceptance. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Sustained release liquid dosage forms suitable for parenteral administration, including, but not limited to, water-in-oil and oil-in-water microemulsions and biodegradable microsphere polymers, may be used according to methods well-known to those having ordinary skill in the art. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. Suitable pharmaceutical carriers are described in Remington's Pharmaceutical Sciences, Mack Publishing Company, a standard reference text in this field. Solubilizing agents may be used to enhance solubility of the compounds of the subject invention in the liquid dosage form. Suitable solubilizing agents include, but are not limited to, amines, amino alcohols, amino sugars, and amino acids, such as glycine, histidine, and lysine.

The compounds of the instant invention may also be administered in intranasal form via use of suitable intranasal vehicles, or via transdermal routes, using those forms of transdermal skin patches well known to those of ordinary skill in that art. To be administered in the form of a transdermal delivery system, the dosage administration will generally be continuous rather than intermittent throughout the dosage regimen.

Parenteral and intravenous forms may also include minerals and other materials to make them compatible with the type of injection or delivery system chosen.

The compounds and compositions of the invention can be coated onto stents for temporary or permanent implantation into the cardiovascular system of a subject.

The compounds of the present invention can be synthesized according to general Schemes.

Variations on the following general synthetic methods will be readily apparent to those skilled in the art and are deemed to be within the scope of the present invention.

In the following Schemes, R₁, R₂, R₃, R₄₃, R₅₀, R′, and R″ refers generally to substituents such as those described herein. Ar₁ and Ar₂ refer generally to bivalent aromatic groups, which may be further substituted using aromatic substitution chemistry well-known to those having ordinary skill in the art. The term “m” is an integer from 0 to 5, “n” is an integer from 2 to 10, and “o” and “p” are each, independently, 0 or 1.

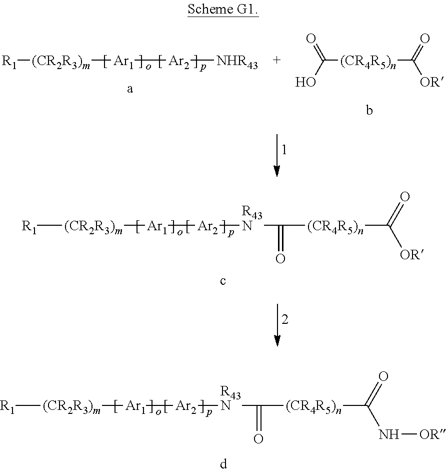

The compounds of the present invention can be synthesized according to general Scheme G1. In step 1 of scheme G1, amine a is coupled to carboxylic acid b using standard amide bond formation chemistry well-known to those having ordinary skill in the art. For example, amine a and carboxylic acid b may be reacted together in the presence of 1-ethyl-3-(3′-dimethylaminopropyl)carbodiimide (EDCI). In step 2, the resulting compound c is converted to the hydroxamic acid or ester d by reaction with, for example, hydroxylamine in the presence of potassium cyanide.

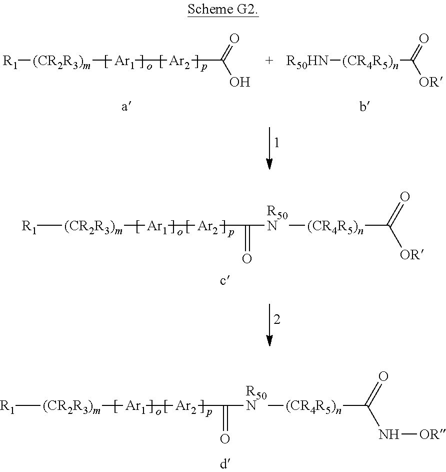

Alternatively, the compounds of the present invention may be synthesized according to general scheme G2. In step 1 of scheme G2, carboxylic acid a′ is coupled to amine b′ using standard amide bond formation chemistry well-known to those having ordinary skill in the art. For example, carboxylic acid a′ and amine b′ may be reacted together in the presence of 1-ethyl-3-(3′-dimethylaminopropyl)carbodiimide (EDCI). In step 2, the resulting compound c′ is converted to the hydroxamic acid or ester d′ by reaction with, for example, hydroxylamine in the presence of potassium cyanide.

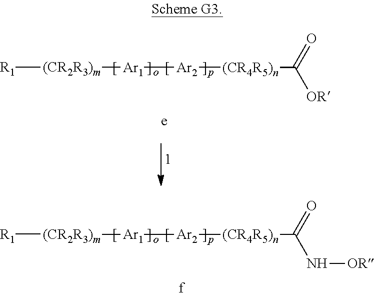

Alternatively, the compounds of the present invention may be synthesized according to general scheme G3. In step 1 of scheme G3, compound e is converted to the hydroxamic acid or ester f by reaction with, for example, hydroxylamine in the presence of potassium cyanide.

The starting compounds contemplated in the present invention may be purchased from commercial sources or may be synthesized using conventional functional group transformations and/or coupling reactions well-known in the chemical arts, for example, those set forth in Organic Synthesis, Michael B. Smith, (McGraw-Hill) Second ed. (2001) and March's Advanced Organic Chemistry: Reactions, Mechanisms, and Structure, Michael B. Smith and Jerry March, (Wiley) Sixth ed. (2007).

Further, where substituents are contemplated, such substituents may be incorporated in the compounds of the present invention using conventional functional group transformations well-known in the chemical arts.

The compounds and compositions of the present invention are useful in the inhibition of histone deacetylases and in the treatment of cancer including, but not limited to, prostate cancer; hematological malignancies including, but limited to, multiple myeloma; inflammatory diseases including, but limited to, rheumatoid arthritis; and neurodegenerative diseases including, but not limited, Alzheimer's disease, Parkinson's disease, Huntington's disease, and Niemann-Pick type C disease. In the structure given below, the substructure to the right of the Ar₁ and Ar₂ mimics the peptide substrate of histone deacetylases:

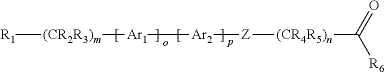

The (C_{R}4R₅) group mimics the backbone structure of a peptide and the R₆, for example when R₆ is —NHOH, mimics the N-terminal of a histone peptide. Z can provide a C-terminal mimic. The activities of the various compounds of the above structure as set forth hereinbelow confirm this structure/activity relationship. Other substructures within the structure above aid aqueous solubility and/or other desired characteristics.

All combinations of the various elements described herein are within the scope of the invention.

Herein, where chemical substituents are disclosed in the alternative, it is intended that each such substituent can be used or combined with one or more other substituents disclosed in the alternative.

This invention will be better understood by reference to the Experimental Details which follow, but those skilled in the art will readily appreciate that the specific experiments detailed are only illustrative of the invention as described more fully in the claims which follow thereafter.

### Experimental Details

### Example 1

### Synthesis of Selective HDAC Inhibitors

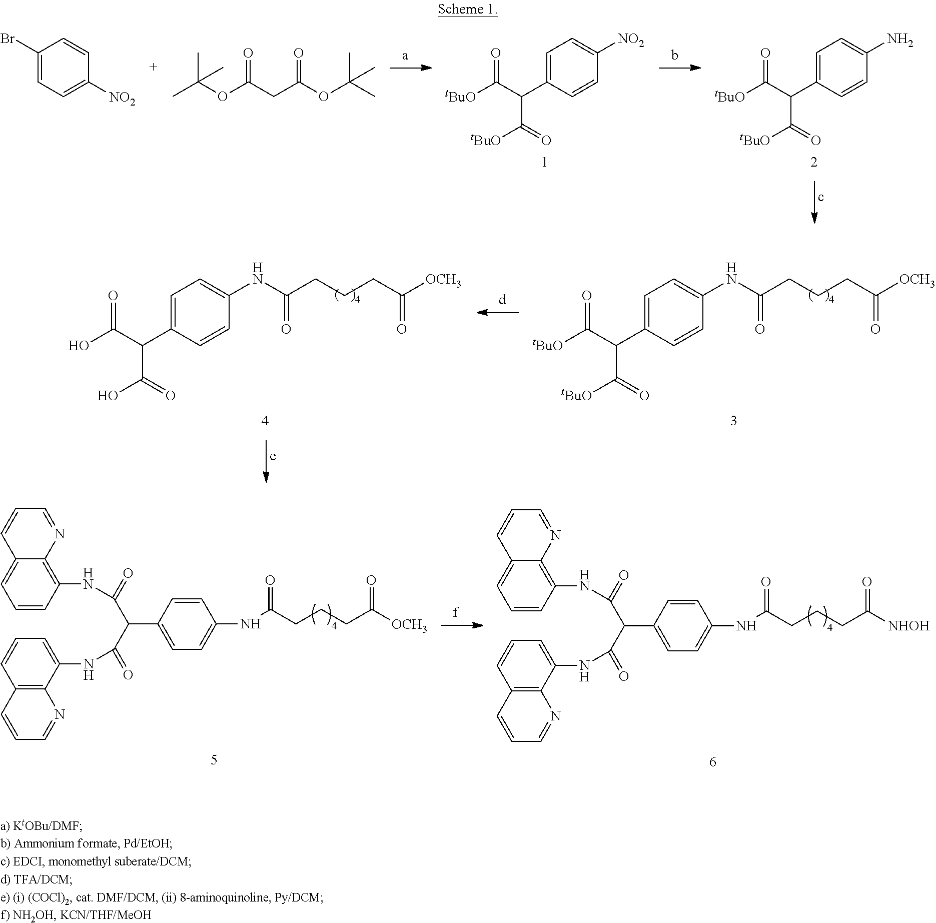

### Di-tert-butyl 2-(4-aminophenyl)malonate (2)

To an ice-cooled solution of di-tert-butyl malonate (24.0 mL, 0.108 mol) in anhydrous DMF (60 mL) was added potassium tert-butoxide (12.1 g, 0.108 mol). The suspension was allowed to warm to room temperature and stirred for 20 min. 1-Bromo-4-nitrobenzene (10.0 g, 0.0495 mol) was added to the mixture and kept at room temperature for 10 min. The resulting yellowish suspension was heated at 120° C. for 1 h. The deep red solution was then quenched with 3N HCl (aq.) and adjusted to pH=5-6. After removing the volatiless in vacuo, EtOAc (200 mL) was added and the solution washed with H₂O (100 mL), brine (50 mL), dried over Na₂SO₄ and filtered. The filtrate was concentrated in vacuo and the residue was dissolved in EtOH (100 mL) and treated with ammonium formate (15.8 g, mol) and Pd/C (10 wt %, 2 g). The suspension was refluxed for 30 min and quenched with celite. After filtration, the filtrate was concentrated and dissolved in EtOAc (200 mL) and washed with H₂O (100 mL), brine (50 mL), dried over Na₂SO₄ and filtered. The filtrate was concentrated in vacuo and the residue was purified by column chromatography on silica gel (EtOAc:Hexanes=1:10-1/1) to give amine 2 (13.8 g, 2-step yield 90.8%) as a pale yellow solid. ¹H NMR (400 MHz, CDCl₃) δ 7.18 (dd, J=2.0 and 6.8 Hz, 1H), 6.68 (dd, J=2.0 and 6.4 Hz, 1H), 4.33 (s, 1H), 3.69 (br, 2H), 1.48 (s, 18H); ¹³C NMR (100 MHz, CDCl₃) δ168.36, 146.49, 130.65, 123.85, 115.43, 82.02, 59.74, 28.32. HR-MS Calcd. for C₁₇H₂₅NO₄ 307.1784. found 307.1793.

### Di-tert-butyl 2-(4-(8-methoxy-8-oxooctanamido)phenyl)malonate (3)

To an ice-cooled solution of 2 (1.25 g, 4.07 mmol) and monomethyl suberate (0.81 mL, 4.48 mmol) in anhydrous CH₂Cl₂ (25 mL) was added 1-ethyl-3-(3′-dimethylaminopropyl)carbodiimide (0.86 g, 4.48 mmol) slowly. The reaction mixture was allowed to warm up to room temperature and stirred for 16 h. The solution was washed with ice-cooled 1N NaOH (aq.) followed by H₂O, brine, dried over Na₂SO₄ and filtered. The filtrate was concentrated in vacuo and the residue was purified by column chromatography on silica gel (EtOAc:Hexanes=1:10-1/1) to give amide 3 (1.75 g, 90.1%). ¹H NMR (400 MHz, CDCl₃) δ 7.52 (d, J=8.0 Hz, 1H), 7.35 (d, J=8.4 Hz, 1H), 7.27 (br, 1H), 4.41 (s, 1H), 3.69 (s, 3H), 2.34 (m, 4H), 1.77-1.64 (m, 4H), 1.50 (s, 18H), 1.41-1.40 (m, 4H); ¹³C NMR (100 MHz, CDCl₃) 0174.63, 172.11, 167.98, 138.52, 130.08, 129.11, 120.09, 82.37, 59.90, 51.08, 37.67, 34.32, 29.16, 28.23, 25.74, 25.09. HR-MS Calcd. for C₂₆H₃₉NO₇ 477.2727. found 477.2736.

### Methyl 8-(4-(1,3-dioxo-1,3-bis(quinolin-8-ylamino)propan-2-yl)phenylamino)-8-oxooctanoate (5)

Compound 3 (1.0 g, 2.1 mmol) in CH₂Cl₂ (4 mL) was treated with trifluoroacetic acid (2 mL, 26 mmol) at room temperature for 19 h. After removing the volatiles, the white solid was suspended in anhydrous CH₂Cl₂ (10 mL). The suspension was treated with oxalyl chloride (0.39 mL, 4.4 mmol) followed by DMF (0.16 mL, 2.1 mmol) at −30° C. to −15° C. for 30 min. The resulting solution was re-cooled to −60° C. and pyridine (0.76 mL, 9.45 mmol) was added followed by 8-aminoquinoline (620 mg, 4.2 mmol). The reaction mixture was allowed to warm up to −30° C. to −20° C. for 30 min before quenching with MeOH (1 mL) at −60° C. The solution was diluted with EtOAc (200 mL) and washed thoroughly with NH₄Cl (sat. aq.), dried over Na₂SO₄ and filtered. The filtrate was concentrated in vacuo and the residue was purified by column chromatography on silica gel (CH₂Cl₂:MeOH=1:40-1/20) to give quinoline derivative 5 (700 mg, 54% from 3). ¹H NMR (400 MHz, CDCl₃) δ 10.98 (br, 2H), 8.89-8.85 (m, 4H), 8.18-7.45 (m, 10H), 7.23 (br, 1H), 4.96 (s, 1H), 3.66 (s, 3H), 2.37-2.29 (m, 4H), 1.73-1.58 (m, 4H), 1.38-1.37 (m, 4H); ¹³C NMR (75 MHz, CDCl₃) δ174.61, 171.59, 167.52, 149.03, 139.23, 138.49, 136.57, 134.68, 130.89, 129.57, 128.31, 127.57, 122.57, 122.04, 120.72, 117.47, 62.57, 51.86, 37.93, 34.30, 29.06, 25.62, 25.03. HR-MS Calcd, for (C₃₆H₃₅N₅O₅+H) 618.2716. found 618.2739.

### N¹-(4-(1,3-Dioxo-1,3-bis(quinolin-8-ylamino)propan-2-yl)phenyl)-N⁸-hydroxyoctanediamide (6)

A suspension of ester 5 (100 mg, 0.162 mmol), hydroxylamine (50% solution in water, 0.6 mL) and a catalytic amount of KCN (0.5 mg) in a co-solvent (MeOH:THF=2 mL:2 mL) was stirred at 35° C. to 40° C. for 24 h. After removing the solvent, the residue was treated with NH₄Cl (sat. aq.) to pH=4-5. The mixture was extracted with a co-solvent (CHCl₃:i-PrOH=4:1), dried over Na₂SO₄ and filtered. The filtrate was concentrated in vacuo and the residue was purified by column chromatography on silica gel (CH₂Cl₂:MeOH=1:40-1/10) to give target hydroxamic acid 6 (40 mg, 40%). ¹H NMR (400 MHz, DMSO-d6) δ 11.05 (br, 2H), 10.32 (br, 1H), 9.95 (br, 1H), 8.94-8.92 (m, 2H), 8.68-8.66 (m, 3H), 8.43-8.40 (m, 2H), 7.73-7.57 (m, 10H), 5.71 (s, 1H), 2.28 (t, J=9.6 Hz, 1H), 1.92 (t, J=9.6 Hz, 1H), 1.56-1.47 (m, 4H), 1.26 (m, 4H); ¹³C NMR (75 MHz, CDCl₃) δ172.14, 169.95, 168.43, 150.00, 139.84, 139.21, 137.48, 135.01, 130.87, 129.77, 128.74, 127.78, 123.43, 123.08, 120.18, 117.96, 60.19, 37.18, 33.09, 29.24, 25.86. HR-MS Calcd. for (C₃₅H₃₄N₆O₅+H) 619.2669. found 619.2690.

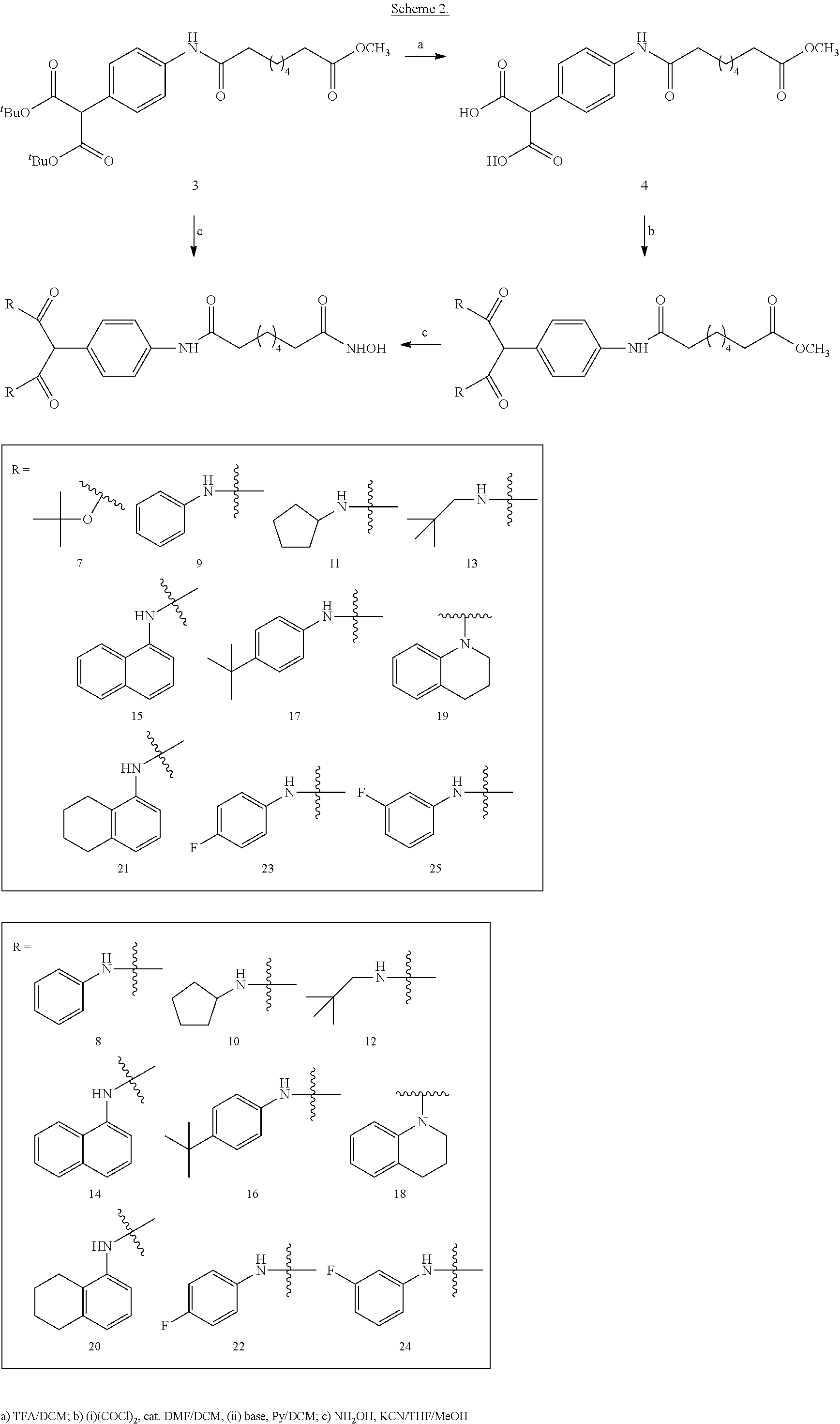

### Di-tert-butyl 2-(4-(8-(hydroxyamino)-8-oxooctanamido)phenyl)malonate (7)

The title compound (130 mg, 86%) was prepared analogously to the procedure for compound 6 described above. ¹H NMR (300 MHz, CDCl₃) δ 9.21 (br, 1H), 8.05 (br, 1H), 7.90 (br, 1H), 7.51 (d, J=8.1 Hz, 2H), 7.31 (d, J=8.4 Hz, 2H), 4.42 (s, 1H), 2.30 (m, 2H), 2.15 (m, 2H), 1.69-1.49 (m, 24H); ¹³C NMR (75 MHz, CDCl₃) δ173.18, 172.21, 168.18, 138.67, 130.10, 129.10, 120.29, 82.61, 66.26, 37.45, 32.95, 28.88, 28.69, 28.28, 25.69, 25.58. HR-MS Calcd. for (C₂₃H₃₈N₂O₇+H) 479.2757. found 479.2768.

### Methyl 8-(4-(1,3-dioxo-1,3-bis(phenylamino)propan-2-yl)phenylamino)-8-oxooctanoate (8)

The title compound (170 mg, 63%) was prepared analogously to the procedure for compound 5 described above. ¹H NMR (400 MHz, DMSO-d6) δ10.18 (br, 2H), 9.88 (br, 1H), 7.62-7.05 (m, 14H), 4.81 (s, 1H), 3.58 (s, 3H), 2.29 (t, J=7.2 Hz, 4H), 1.58-1.53 (m, 4H), 1.29 (m, 4H); ¹³C NMR (75 MHz, CDCl₃) δ 174.20, 72.06, 167.64, 139.72, 139.62, 139.52, 130.79, 130.14, 129.60, 124.39, 120.22, 119.73, 60.22, 52.02, 37.15, 34.08, 29.15, 29.08, 25.80, 25.17. HR-MS Calcd. for (C₃₀H₃₃N₃O₅+H) 516.2498. found 516.2487.

### N¹-(4-(1,3-Dioxo-1,3-bis(phenylamino)propan-2-yl)phenyl)-N⁸-hydroxyoctanediamide (9)

The title compound (120 mg, 86%) was prepared analogously to the procedure for compound 6 described above. ¹H NMR (400 MHz, DMSO-d6) δ 10.33 (br, 1H), 10.19 (br, 2H), 9.91 (br, 1H), 8.67 (br, 1H), 7.61-7.05 (m, 14H), 4.81 (s, 1H), 3.58 (s, 3H), 2.29 (t, J=7.2 Hz, 2H), 1.93 (t, J=7.2 Hz, 2H), 1.57-1.42 (m, 4H), 1.10 (m, 4H); ¹³C NMR (75 MHz, CDCl₃) δ 173.69, 171.97, 168.44, 138.82, 138.35, 131.15, 128.97, 128.89, 124.66, 120.50, 59.63, 36.84, 32.68, 28.88, 28.81, 25.66, 25.57. HR-MS Calcd. for (C₂₉H₃₂N₄O₅+H) 517.2451. found 517.2436.

### Methyl 8-(4-(1,3-bis(cyclopentylamino)-1,3-dioxopropan-2-yl)phenylamino)-8-oxooctanoate (10)

The title compound (90 mg, 35%) was prepared analogously to the procedure for compound 5 described above. ¹H NMR (300 MHz, CDCl₃) δ 7.56 (br, 1H), 7.46-7.28 (m, 4H), 7.06-7.03 (br, 2H), 4.20-4.14 (m, 3H), 3.68 (s, 3H), 2.38-2.31 (m, 4H), 1.98-1.38 (m, 24H); ¹³C NMR (100 MHz, CDCl₃) δ 174.63, 172.42, 169.66, 138.19, 131.62, 128.62, 121.02, 58.22, 51.89, 51.72, 37.55, 34.40, 33.12, 29.28, 25.84, 25.16, 24.16. HR-MS Calcd. for (C₂₈H₄₁N₃O₅+H) 500.3124. found 500.3131.

### N¹-(4-(1,3-Bis(cyclopentylamino)-1,3-dioxopropan-2-yl)phenyl)-N⁸-hydroxyoctanedamide (11)

The title compound (45 mg, 56%) was prepared analogously to the procedure for compound 6 described above. ¹H NMR (300 MHz, DMSO-d6) δ 10.31 (br, 1H), 9.85 (br, 1H), 8.66 (br, 1H), 8.17 (br, 1H), 8.14 (s, 1H), 7.51 (d, J=8.4 Hz, 2H), 7.25 (d, J=8.7 Hz, 2H), 4.23 (s, 1H), 4.30-3.94 (m, 2H), 2.28 (t, J=6.9 Hz, 2H), 1.94 (t, J=7.2 Hz, 2H), 1.77-1.36 (m, 24H); ¹³C NMR (75 MHz, DMSO-d6) δ 172.03, 169.94, 168.99, 139.20, 132.00, 128.98, 119.82, 58.00, 51.30, 37.17, 33.06, 29.25, 25.89, 24.24. HR-MS Calcd. for (C₂₇H₄₀N₄O₅+H) 501.3077. found 501.3069.

### Methyl 8-(4-(1,3-bis(neopentylamino)-1,3-dioxopropan-2-yl)phenylamino)-8-oxooctanoate (12)

The title compound (60 mg, 22%) was prepared analogously to the procedure for compound 5 described above. ¹H NMR (300 MHz, CDCl₃) 6.7.89 (br, 1H), 7.39-7.32 (m, 5H), 4.31 (s, 1H), 3.68 (s, 3H), 3.07 (d, J=5.7 Hz, 4H), 2.35-2.30 (m, 4H), 1.73-1.63 (m, 4H), 1.39-1.38 (m, 4H), 0.87 (s, 18H); ¹³C NMR (75 MHz, CDCl₃) δ 174.40, 172.42, 170.40, 138.22, 131.57, 128.80, 121.03, 58.29, 51.87, 51.18, 37.55, 34.38, 32.41, 29.31, 27.61, 25.80, 25.13. HR-MS Calcd. for (C₂₈H₄₅N₃O₅+H) 504.3437. found 504.3425.

### N¹-(4-(1,3-bis(neopentylamino)-1,3-dioxopropan-2-yl)phenyl)-N⁸-hydroxyoctanediamide (13)

The title compound (25 mg, 50%) was prepared analogously to the procedure for compound 6 described above. ¹H NMR (400 MHz, DMSO-d6) δ 10.32 (br, 1H), 9.85 (br, 1H), 8.65 (br, 1H), 8.26-8.23 (m, 2H), 7.53 (d, J=8.4 Hz, 2H), 7.33 (d, J=8.8 Hz, 2H), 4.44 (s, 1H), 2.94 (d, J=6.4 Hz, 2H), 2.28 (t, J=7.2 Hz, 2H), 1.94 (t, J=7.2 Hz, 2H), 1.57-1.49 (m, 4H), 1.28 (m, 4H), 0.87 (s, 18H); ¹³C NMR (75 MHz, CDCl₃) δ 172.04, 169.94, 139.28, 132.16, 128.97, 119.78, 58.15, 50.05, 37.18, 33.10, 32.89, 29.67, 27.91, 25.87. HR-MS Calcd. for (C₂₇H₄₄N₄O₅+H) 505.3390. found 505.3368.

### Methyl 8-(4-(1,3-bis(naphthalen-1-ylamino)-1,3-dioxopropan-2-yl)phenylamino)-8-oxooctanoate (14)

The title compound (200 mg, 40%) was prepared analogously to the procedure for compound 5 described above. ¹H NMR (300 MHz, CDCl₃) δ 9.78 (br, 2H), 7.90-7.36 (m, 19H), 4.98 (s, 1H), 3.64 (s, 3H), 2.32-2.25 (m, 4H), 1.67-1.56 (m, 4H), 1.31 (m, 4H); ¹³C NMR (75 MHz, CDCl₃ with drops of DMSO-d6) δ 173.12, 171.25, 168.24, 138.33, 133.23, 131.88, 130.14, 127.66, 126.59, 125.54, 125.30, 124.94, 124.82, 120.92, 119.86, 119.32, 57.62, 50.55, 36.25, 33.09, 28.01, 24.56, 23.91. HR-MS Calcd. for (C₃₈H₃₇N₃O₅+H) 616.2811. found 616.2797.

### N¹-(4-(1,3-bis(naphthalen-1-ylamino)-1,3-dioxopropan-2-yl)phenyl)-N⁸-hydroxyoctanediamide (15)

The title compound (30 mg, 38%) was prepared analogously to the procedure for compound 6 described above. ¹H NMR (300 MHz, DMSO-d6) δ 10.61 (br, 2H), 10.31 (br, 1H), 9.94 (br, 1H), 8.64 (br, 1H), 8.00-7.48 (m, 18H), 5.15 (s, 1H), 2.28 (d, J=7.2 Hz, 2H), 1.91 (t, J=7.2 Hz, 2H), 1.56-1.46 (m, 4H), 1.10-1.05 (m, 4H); ¹³C NMR (75 MHz, CDCl₃) δ 172.14, 169.94, 169.10, 139.72, 134.56, 133.88, 131.32, 129.75, 129.14, 128.39, 127.00, 126.45, 123.05, 122.10, 120.08, 59.08, 37.22, 33.10, 29.27, 25.89. HR-MS Calcd. for (C₃₇H₃₆N₄O₅+H) 617.2764. found 617.2781.

### Methyl 8-(4-(1,3-bis(4-tert-butylphenylamino)-1,3-dioxopropan-2-yl)phenylamino)-8-oxooctanoate (16)

The title compound (190 mg, 55%) was prepared analogously to the procedure for compound 5 described above. ¹H NMR (400 MHz, CDCl₃) δ 9.34 (br, 2H), 7.70 (br, 1H), 7.47-7.33 (m, 12H), 4.59 (s, 1H), 3.67 (s, 3H), 2.37-2.29 (m, 4H), 1.72-1.31 (m, 26H); ¹³C NMR (75 MHz, CDCl₃ with drops of MeOH-d4) δ 177.09, 175.11, 170.37, 150.00, 140.54, 136.98, 133.02, 130.38, 127.90, 122.61, 122.37, 60.89, 53.69, 39.16, 36.50, 36.11, 33.37, 30.89, 27.58, 26.83. HR-MS Calcd. for (C₃₈H₄₉N₃O₅+H) 628.3750. found 628.3734.

### N¹-(4-(1,3-bis(4-tert-butylphenylamino)-1,3-dioxopropan-2-yl)phenyl)-N⁸-hydroxyoctanediamide (17)

The title compound (90 mg, 64%) was prepared analogously to the procedure for compound 6 described above. ¹H NMR (400 MHz, DMSO-d6) δ 10.31 (br, 1H), 10.11 (br, 2H), 9.88 (br, 1H), 8.63 (br, 1H), 7.58 (d, J=8.4 Hz, 2H), 7.7.52 (d, J=8.4 Hz, 4H), 7.36 (d, J=8.4 Hz, 2H), 7.33 (d, J=8.4 Hz, 4H), 4.76 (s, 1H), 2.29 (t, J=7.6 Hz, 2H), 1.94 (t, J=7.6 Hz, 2H), 1.57-1.49 (m, 4H), 1.26 (m, 22H); ¹³C NMR (75 MHz, CDCl₃ with drops of MeOH-d4) δ 173.05, 171.37, 168.12, 147.78, 138.21, 134.68, 130.82, 128.14, 125.64, 120.39, 120.13, 58.56, 36.73, 34.25, 32.49, 31.13, 28.28, 28.19, 25.14, 24.99. HR-MS Calcd. for (C₃₇H₄₈N₄O₅+H) 629.3703. found 629.3781.

### Methyl 8-(4-(1,3-bis(3,4-dihydroquinolin-1(2H)-yl)-1,3-dioxopropan-2-yl)phenylamino)-8-oxooctanoate (18)

The title compound (320 mg, 65%) was prepared analogously to the procedure for compound 5 described above. ¹H NMR (400 MHz, CDC₃) δ 7.44-7.35 (m, 4H), 7.09 (br, 8H), 5.63 (br, 1H), 3.87 (br, 2H), 3.69 (s, 3H), 3.52 (br, 2H), 2.54 (br, 4H), 2.38-2.32 (m, 4H), 1.80-1.63 (m, 8H), 1.41-1.39 (m, 4H); ¹³C NMR (75 MHz, CDCl₃) δ 174.66, 172.26, 168.65, 139.04, 138.40, 129.95, 129.01, 126.48, 124.77, 120.61, 43.89, 37.58, 34.42, 31.32, 29.30, 26.79, 25.76, 25.20, 24.08. HR-MS Calcd. for (C₃₆H₄₁N₃O₅+H) 596.3124. found 596.3107.

### N¹-(4-(1,3-bis(3,4-dihydroquinolin-1(2H)-yl)-1,3-dioxopropan-2-yl)phenyl)-N⁸-hydroxyoctanediamide (19)

The title compound (70 mg, 50%) was prepared analogously to the procedure for compound 6 described above. ¹H NMR (400 MHz, CDCl₃) δ 10.03 (br, 1H), 8.85 (br, 2H), 7.45 (br, 2H), 7.08 (br, 10H), 5.66 (br, 1H), 4.00 (br, 2H), 3.45 (br, 2H), 2.50-1.33 (m, 20H); ¹³C NMR (75 MHz, CDCl₃) δ 173.50, 171.98, 168.80, 138.74, 130.01, 129.03, 126.54, 124.70, 120.49, 43.92, 37.36, 32.96, 30.10, 28.88, 28.75, 26.77, 25.76, 25.64, 24.04. HR-MS Calcd. for (C₃₅H₄₀N₄O₅+H) 597.3077. found 597.3088.

### Methyl 8-(4-(1,3-dioxo-1,3-bis(5,6,7,8-tetrahydronaphthalen-1-ylamino)propan-2-yl)phenylamino)-8-oxooctanoate (20)

The title compound (200 mg, 58%) was prepared analogously to the procedure for compound 5 described above. ¹H NMR (300 MHz, CDCl₃) δ 8.77 (br, 2H), 7.64-7.45 (m, 6H), 7.10 (t, J=7.8 Hz, 2H), 6.93 (d, J=7.8 Hz, 2H), 4.64 (s, 1H), 2.77 (t, J=6.0 Hz, 4H), 2.55 (t, J=5.7 Hz, 4H), 2.38-2.30 (m, 4H), 1.81-1.74 (m, 8H), 1.40-1.38 (m, 4H); ¹³C NMR (75 MHz, CDCl₃) δ 174.73, 172.31, 168.50, 138.66, 138.52, 135.19, 129.95, 129.08, 127.23, 125.97, 121.21, 121.10, 59.27, 51.93, 37.69, 34.35, 30.18, 29.17, 25.69, 25.10, 24.92, 23.19, 22.86. HR-MS Calcd. for (C₃₈H₄₅N₃O₅+H) 624.3437. found 624.3455.

### N¹-(4-(1,3-dioxo-1,3-bis(5,6,7,8-tetrahydronaphthalen-1-ylamino)propan-2-yl)phenyl)-N⁸-hydroxyoctanediamide (21)

The title compound (40 mg, 58%) was prepared analogously to the procedure for compound 6 described above. ¹H NMR (400 MHz, DMSO-d6) δ 10.31 (br, 1H), 9.90 (br, 3H), 8.63 (br, 1H), 7.61 (d, J=8.4 Hz, 2H), 7.46 (d, J=8.4 Hz, 2H), 7.38 (d, J=7.2 Hz, 2H), 7.07 (t, J=7.2 Hz, 2H), 6.91 (d, J=7.2 Hz, 2H), 4.81 (s, 1H), 2.74 (s, 4H), 2.52 (m, 4H), 2.30 (m, 2H), 1.93 (m, 2H), 1.71-1.49 (m, 12H), 1.29 (m, 4H); ¹³C NMR (75 MHz, DMSO-d6) δ 172.13, 169.95, 168.44, 139.67, 138.44, 136.26, 131.45, 130.64, 129.16, 127.02, 126.14, 122.08, 120.09, 58.79, 37.20, 33.10, 30.05, 29.27, 25.89, 24.94, 23.24, 23.03. HR-MS Calcd. for (C₃₇H₄₈N₄O₅+H) 625.3390. found 625.3395.

### Methyl 8-(4-(1,3-bis(4-fluorophenylamino)-1,3-dioxopropan-2-yl)phenylamino)-8-oxooctanoate (22)

The title compound (190 mg, 63%) was prepared analogously to the procedure for compound 5 described above. ¹H NMR (300 MHz, DMSO-d6) δ 10.21 (br, 2H), 9.88 (br, 1H), 7.61-7.10 (m, 12H), 4.75 (s, 1H), 3.55 (s, 3H), 2.67 (t, J=7.5 Hz, 4H), 1.54-1.50 (m, 4H), 1.26 (s, 4H); ¹³C NMR (75 MHz, MeOH-d4 with drops of DMSO-d6) δ 177.23, 175.36, 170.44, 161.92 (d, J=242.5 Hz), 140.81, 135.85, 132.80, 130.56, 124.53, 122.85, 117.77 (d, J=22.4 Hz), 60.95, 53.79, 39.25, 36.20, 31.01, 27.71, 26.94. HR-MS Calcd. for (C₃₀H₃₁N₃O₅F₂+H) 552.2310. found 552.2334.

### N¹-(4-(1,3-bis(4-fluorophenylamino)-1,3-dioxopropan-2-yl)phenyl)-N⁸-hydroxyoctanediamide (23)

The title compound (30 mg, 50%) was prepared analogously to the procedure for compound 6 described above. ¹H NMR (400 MHz, DMSO-d6) δ 10.29 (br, 1H), 10.19 (br, 2H), 9.86 (br, 1H), 8.61 (br, 1H), 7.61-7.54 (m, 6H), 7.33 (d, J=8.4 Hz, 2H), 7.12 (t, J=8.4 Hz, 2H), 4.76 (s, 1H), 2.26 (t, J=7.2 Hz, 2H), 1.91 (t, J=7.2 Hz, 2H), 1.56-1.45 (m, 4H), 1.26 (m, 4H); ¹³C NMR (75 MHz, DMSO-d6) δ 172.13, 170.00, 167.52, 158.97 (d, J=238.6 Hz), 139.53, 136.12, 130.63, 130.21, 122.02 (d, J=31.8 Hz), 119.75, 116.16 (d, J=22.1 Hz), 60.09, 39.49, 33.09, 29.24, 25.89. HR-MS Calcd. for (C₂₉H₃₀N₄O₅F₂+H) 553.2263. found 553.2271.

### Methyl 8-(4-(1,3-bis(3-fluorophenylamino)-1,3-dioxopropan-2-yl)phenylamino)-8-oxooctanoate (24)

The title compound (150 mg, 80%) was prepared analogously to the procedure for compound 5 described above. ¹H NMR (400 MHz, CDCl₃) δ 9.57 (br, 2H), 7.69 (br, 1H), 7.52-7.18 (m, 10H), 6.84 (t, J=8.4 Hz, 2H), 4.59 (s, 1H), 3.68 (s, 3H), 2.37 (t, J=7.6 Hz, 2H), 2.31 (t, J=7.2 Hz, 2H), 1.74-1.64 (m, 4H), 1.37-1.36 (m, 4H); ¹³C NMR (75 MHz, CDCl₃ with drops of MeOH-d4) δ 175.18, 173.18 (d, J=6.6 Hz), 168.54 (d, J=6.4 Hz), 163.17 (d, J=243.2 Hz), 139.38 (t, J=7.0 Hz), 138.78 (d, J=6.0 Hz), 130.58, 130.39 (d, J=9.2 Hz), 128.62, 121.02 (d, J=7.7 Hz), 116.02, 111.79 (d, J=21.2 Hz), 108.10 (dd, J=6.4 and 26.2 Hz), 59.15, 51.91, 37.40, 34.26, 29.04, 29.00, 25.70, 24.96. HR-MS Calcd. for (C₃₀H₃₁N₃O₅F₂+H) 552.2310. found 552.2335.

### N¹-(4-(1,3-bis(3-fluorophenylamino)-1,3-dioxopropan-2-yl)phenyl)-N⁸-hydroxyoctanediamide (25)

The title compound (60 mg, 50%) was prepared analogously to the procedure for compound 6 described above. ¹H NMR (400 MHz, DMSO-d6) δ 10.36 (br, 2H), 10.30 (br, 1H), 9.87 (br, 1H), 8.62 (br, 1H), 7.59-7.29 (m, 10H), 6.86 (t, J=8.8 Hz, 2H), 4.82 (s, 1H), 2.27 (t, J=7.2 Hz, 2H), 1.92 (t, J=7.2 Hz, 2H), 1.57-1.46 (m, 4H), 1.26 (m, 4H); ¹³C NMR (75 MHz, DMSO-d6) δ 172.13, 170.00, 167.78, 162.96 (d, J=240.0 Hz), 141.50 (d, J=11.0 Hz), 139.60, 131.26 (d, J=9.3 Hz), 130.36, 130.20, 119.75, 115.89, 110.82 (d, J=21.0 Hz), 106.94 (d, J=26.2 Hz), 60.43, 39.50, 37.20, 33.09, 29.25, 25.89. HR-MS Calcd. for (C₂₉H₃₀N₄O₅F₂+H) 553.2263. found 553.2273.

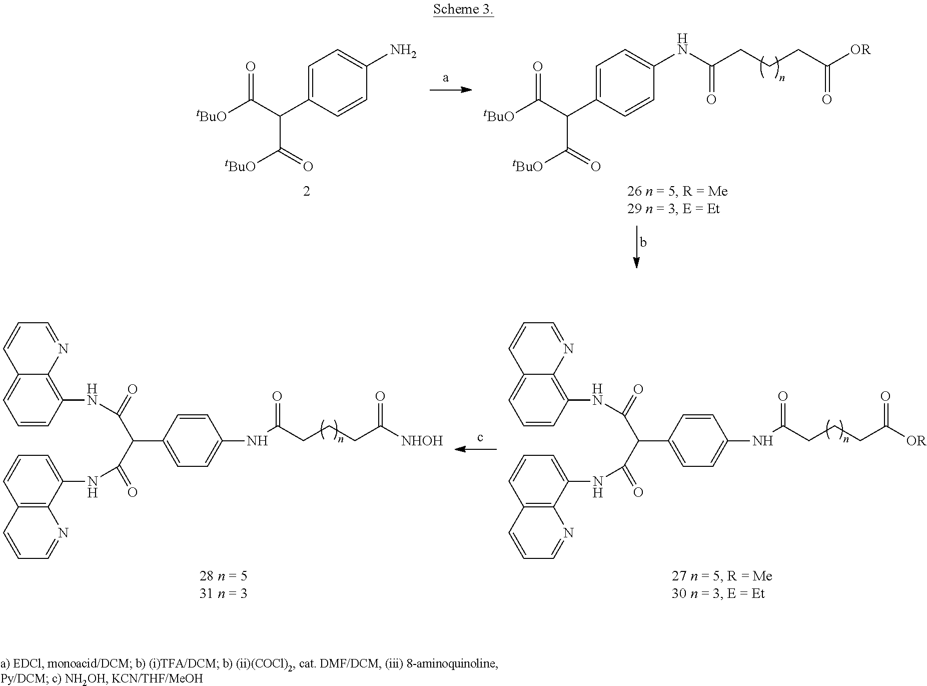

### Di-tert-butyl 2-(4-(9-methoxy-9-oxononanamido)phenyl)malonate (26)

The title compound (195 mg, 64%) was prepared analogously to the procedure for compound 3 described above. ¹H NMR (400 MHz, CDCl₃) 17.53 (d, J=8.4 Hz, 2H), 7.36 (d, J=8.4 Hz, 2H), 7.17 (br, 1H), 4.41 (s, 1H), 3.69 (s, 3H), 2.38-2.31 (m, 4H), 1.76-1.38 (m, 28H); ¹³C NMR (75 MHz, DMSO-d6) δ 174.74, 171.99, 167.96, 138.34, 130.20, 129.29, 120.05, 82.41, 59.93, 51.85, 37.92, 34.42, 29.36, 29.31, 29.28, 28.27, 25.86, 25.23. HR-MS Calcd. for (C₂₇H₄₁NO₇) 491.2883. found 491.2874.

### Methyl 9-(4-(1,3-dioxo-1,3-bis(quinolin-8-ylamino)propan-2-yl)phenylamino)-9-oxononanoate (27)

The title compound (100 mg, 38%) was prepared analogously to the procedure for compound 5 described above. ¹H NMR (400 MHz, CDCl₃) δ 10.98 (br, 2H), 8.89-8.85 (m, 4H), 8.18-7.46 (m, 12H), 7.22 (br 1H), 4.97 (s, 1H), 2.37-2.29 (m, 4H), 1.74-1.61 (m, 4H), 1.35 (m, 4H); ¹³C NMR (75 MHz, DMSO-d6) δ 174.69, 171.68, 167.53, 149.03, 139.21, 138.52, 136.58, 134.66, 130.84, 129.55, 128.31, 127.56, 122.59, 122.05, 120.74, 117.48, 62.54, 51.88, 38.07, 34.40, 29.25, 25.77, 25.19. HR-MS Calcd. for (C₃₇H₃₇N₅O₅+H) 632.2873. found 632.2880.

### N¹-(4-(1,3-dioxo-1,3-bis(quinolin-8-ylamino)propan-2-yl)phenyl)-N⁹-hydroxynonanediamide (28)

The title compound (50 mg, 50%) was prepared analogously to the procedure for compound 6 described above. ¹H NMR (400 MHz, DMSO-d6) δ 11.04 (br, 2H), 10.31 (br, 1H), 9.93 (br, 1H), 8.94-8.93 (m, 2H), 8.68 (d, J=7.6 Hz, 2H 2H), 8.64 (br, 1H), 8.42 (dd, J=1.6 and 8.4 Hz, 2H), 7.7.2-7.58 (m, 12H), 5.72 (s, 1H), 2.29 (t, J=7.2 Hz, 2H), 1.93 (t, J=7.2 Hz, 2H), 1.57-1.27 (m, 10H); ¹³C NMR (75 MHz, DMSO-d6) δ 172.18, 169.96, 168.49, 150.00, 139.90, 139.21, 137.48, 135.06, 130.88, 129.77, 128.74, 127.78, 123.43, 123.08, 120.21, 117.97, 60.18, 33.10, 29/36, 25.93. HR-MS Calcd. for (C₃₇H₃₇N₅O₅+H) 633.2825. found 633.2802.

### Di-tert-butyl 2-(4-(7-ethoxy-7-oxoheptanamido)phenyl)malonate (29)

The title compound (228 mg, 61%) was prepared analogously to the procedure for compound 3 described above. ¹H NMR (400 MHz, CDCl₃) δ 7.54-7.35 (m, 4H), 7.23 (br, 1H), 4.41 (s, 1H), 4.15 (q, J=7.2 Hz, 2H), 2.40-2.32 (m, 4H), 1.77-1.44 (m, 24H), 1.27 (t, J=7.2 Hz, 3H); ¹³C NMR (75 MHz, DMSO-d6) δ 174.18, 171.97, 167.99, 138.45, 130.11, 129.18, 120.11, 82.39, 60.64, 59.91, 37.45, 34.46, 28.98, 28.24, 25.54, 24.94, 14.59. HR-MS Calcd. for (C₂₆H₃₉NO₇) 477.2727. found 477.2715.

### Ethyl 7-(4-(1,3-dioxo-1,3-bis(quinolin-8-ylamino)propan-2-yl)phenylamino)-7-oxoheptanoate (30)

The title compound (205 mg, 64%) was prepared analogously to the procedure for compound 5 described above. ¹H NMR (400 MHz, CDCl₃) δ 10.98 (br, 2H), 8.88-8.84 (m, 4H), 8.17-7.40 (m, 13H), 4.97 (s, 1H), 4.12 (q, J=7.2 Hz, 2H), 2.36 (t, J=7.2 Hz, 2H), 2.31 (t, J=7.6 Hz, 2H), 1.76-1.64 (m, 4H), 1.42-1.38 (m, 2H), 1.23 (t, J=7.2 Hz, 3H); ¹³C NMR (75 MHz, DMSO-d6) δ 174.23, 171.54, 167.47, 149.06, 139.17, 138.45, 136.60, 134.54, 130.76, 130.36, 129.54, 128.32, 127.55, 122.64, 122.06, 120.68, 117.48, 62.43, 60.71, 37.66, 34.41, 28.89, 25.45, 24.78, 14.60. HR-MS Calcd. for (C₃₆H₃₅N₅O₅+H) 618.2716. found 618.2693.

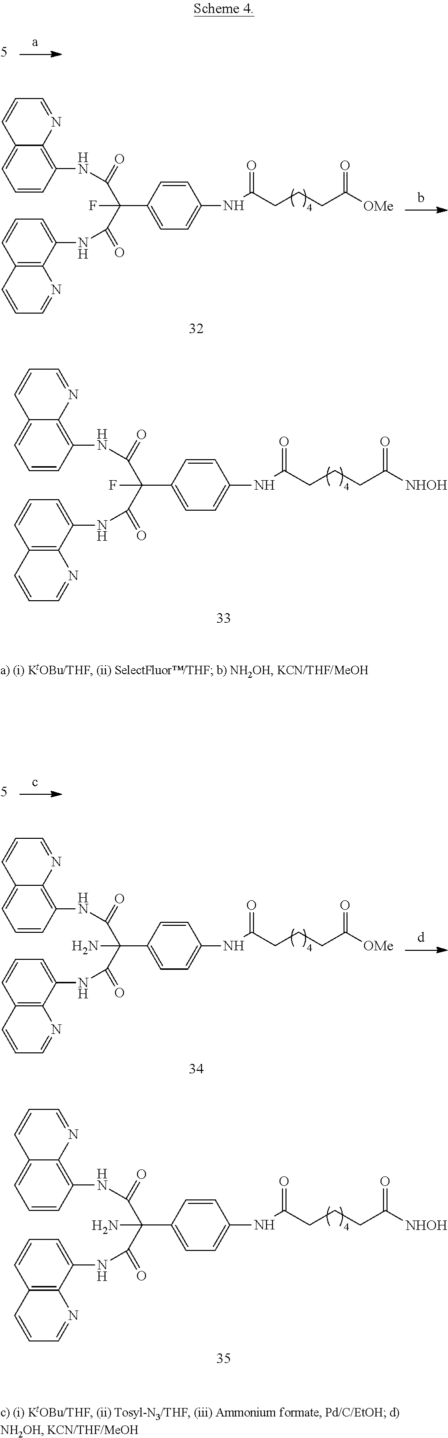

### N¹-(4-(1,3-dioxo-1,3-bis(quinolin-8-ylamino)propan-2-yl)phenyl)-N⁷-hydroxyheptanediamide (31)

The title compound (30 mg, 51%) was prepared analogously to the procedure for compound 6 described above. ¹H NMR (400 MHz, DMSO-d6) δ11.04 (br, 2H), 10.31 (br, 1H), 9.94 (br, 1H), 8.94-8.40 (m, 7H), 7.73-6.58 (m, 10H), 5.71 (s, 1H), 2.28 (t, J=7.2 Hz, 2H), 2.31 (t, J=6.9 Hz, 2H), 1.58-1.47 (m, 4H), 1.27-1.23 (m, 2H); ¹³C NMR (75 MHz, DMSO-d6) δ 172.10, 169.89, 168.49, 150.02, 139.89, 139.21, 137.45, 135.05, 130.89, 129.76, 128.74, 127.78, 123.44, 123.09, 120.21, 117.97, 60.17, 37.07, 33.00, 29.07, 25.77, 25.67. HR-MS Calcd. for (C₃₆H₃₅N₅O₅+H) 605.2512. found 605.2505.

### Methyl 8-(4-(2-fluoro-1,3-dioxo-1,3-bis(quinolin-8-ylamino)propan-2-yl)phenylamino)-8-oxooctanoate (32)

A solution of 2 (200 mg, 0.32 mmol) in THF was treated with potassium tert-butoxide (40 mg, 0.35 mmol) at room temperature and the yellowish suspension was then cooled to −78° C. SelectFluor™ (130 mg, 0.35 mmol) in CH₃CN (10 mL) was added and the reaction was allowed to warm up to room temperature for 15 min. The reaction mixture was poured into water (10 mL) and extracted with CH₂Cl₂ (10 mL×2). Then the organic phase was combined and dried over Na₂SO₄ and filtered. The filtrate was concentrated in vacuo and the residue was purified by column chromatography on silica gel (CH₂Cl₂:MeOH=1:80-1:40) to give desired compound 32 (180 mg, 87%). ¹H NMR (400 MHz, CDCl₃) δ 11.41 (br, 1H), 11.40 (br, 1H), 8.91-8.85 (m, 4H), 8.17-7.46 (m, 13H), 3.66 (s, 3H), 2.34 (t, J=7.2 Hz, 2H), 2.28 (t, J=7.6 Hz, 2H), 1.71-1.58 (m, 4H), 1.34-1.28 (m, 4H); ¹³C NMR (75 MHz, CDCl₃) δ 174.67, 171.92, 164.63 (d, J=23.5 Hz), 149.25, 139.94, 139.31, 136.57, 133.90, 130.62 (d, J=22.2 Hz), 128.34, 127.46, 127.38, 127.28, 123.22, 122.23, 120.15, 117.64, 95.99 (d, J=199.7 Hz), 51.87, 37.84, 34.30, 29.06, 25.56, 25.03; ¹⁹F-NMR (283 MHz, CDCl₃) δ −150.51. HR-MS Calcd. for (C₃₆H₃₄N₅O₅F+H) 636.2622. found 636.2643.

### N¹-(4-(2-fluoro-1,3-dioxo-1,3-bis(quinolin-8-ylamino)propan-2-yl)phenyl)-N⁸-hydroxyoctanediamide (33)

The title compound (15 mg, 19%) was prepared analogously to the procedure for compound 6 described above. ¹H NMR (400 MHz, DMSO-d6) δ 11.22 (br, 2H), 10.32 (br, 1H), 10.12 (br, 1H), 8.98 (d, J=4.2 Hz, 2H), 8.70 (d, J=7.8 Hz, 2H), 8.65 (br, 1H), 8.48 (d, J=8.4 Hz, 2H), 7.82-7.64 (m, 10H), 2.29 (m, 1H), 1.91 (t, J=6.9 Hz, 2H), 1.55-1.46 (m, 4H), 1.26 (m, 4H); ¹³C NMR (75 MHz, CDCl₃) δ 172.79, 170.44, 164.46 (d, J=23.9 Hz), 150.54, 141.59, 138.84, 137.68, 133.34, 129.88, 129.58, 128.66, 127.82, 124.44, 123.48, 120.22, 117.69, 96.41 (d, J=198.00 Hz), 37.09, 32.99, 29.07, 25.71; ¹⁹F-NMR (283 MHz, CDCl₃) 8-144.61. HR-MS Calcd. for (C₃₅H₃₃N₆O₅F+H) 637.2575. found 637.2568.

### Methyl 8-(4-(2-amino-1,3-dioxo-1,3-bis(quinolin-8-ylamino)propan-2-yl)phenylamino)-8-oxooctanoate (34)

Compound 5 (460 mg, 0.75 mmol) was suspended in the THF (15 mL) and treated with potassium tert-butoxide (91 mg, 0.79 mmol) at −78° C. The reaction mixture was allowed to warm up to room temperature for 5 min and then re-cooled to 78° C. Tosyl azide (510 mg, 2.6 mmol) in THF (5 mL) was added portion wise. The reaction was allowed to warm up to room temperature for 30 min. The reaction was re-cooled to −78° C. and quenched with HOAc (1 mL) and warmed up to room temperature for 1 h. The mixture was diluted with EtOAc and washed with H₂O. The organic layer was dried over Na₂SO₄ and filtered. The filtrate was concentrated in vacuo and the residue was directly dissolved in EtOH (10 mL) and treated with ammonium formate (470 mg, 7.5 mmol) and Pd/C (200 mg) at refluxed temperature for 30 min. Celite was added to the reaction mixture and the suspension was stirred for 15 min. and filtered. The filtrate was evaporated to dryness and dissolved in a co-solvent (CHCl₃:i-PrOH=4:1) and the solution was washed with H₂O, dried over Na₂SO₄ and filtered. The filtrate was concentrated in vacuo and the residue was purified by column chromatography on silica gel (CH₂Cl₂:MeOH=1:40-1:15) to give desired compound 34 (300 mg, 64%). ¹H NMR (300 MHz, CDCl₃) δ 12.28 (br, 1H), 8.93-8.88 (m, 4H), 8.16-8.13 (m, 2H), 7.81-7.78 (m, 2H), 7.59-7.27 (m, 10H), 4.97 (br, 2H), 3.89 (s, 3H), 2.31-2.24 (m, 4H), 1.65-1.43 (m, 4H), 0.90 (m, 4H); HR-MS Calcd. for (C₃₆H₃₆N₆O₅+H) 633.2825. found 633.2852.

### N¹-(4-(2-amino-1,3-dioxo-1,3-bis(quinolin-8-ylamino)propan-2-yl)phenyl)-N⁸-hydroxyoctanediamide (35)

The title compound (15 mg, 50%) was prepared analogously to the procedure for compound 6 described above. ¹H NMR (300 MHz, DMSO-d6) δ 12.15 (br, 2H), 10.30 (s, 1H), 9.94 (s, 1H), 8.97 (d, J=2.7 Hz, 2H), 8.82 (d, J=6.0 Hz, 2H), 8.63 (s, 1H), 8.43 (d, J=8.4 Hz, 2H), 7.75-7.46 (m, 10H), 3.48 (br, 2H), 2.25 (t, J=7.5 Hz, 2H), 1.89 (t, J=7.5 Hz, 2H), 1.45 (m, 4H), 1.24 (m, 4H); HR-MS Calcd. for (C₃₅H₃₅N₇O₅+H) 634.2778. found 634.2793.

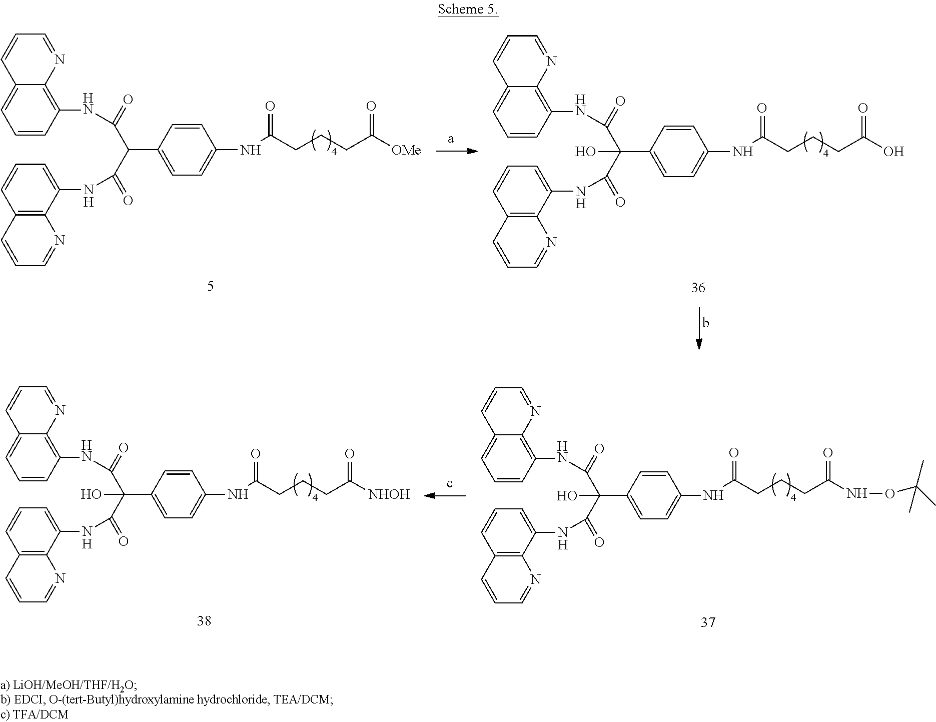

### 8-(4-(2-Hydroxy-1,3-dioxo-1,3-bis(quinolin-8-ylamino)propan-2-yl)phenylamino)-8-oxooctanoic acid (36)

A solution of 5 (150 mg, 0.24 mmol) in a co-solvent (MeOH/THF/H₂O=1/6/6, 5 mL) was treated with LiOH.H₂O (60.4 mg, 1.44 mmol) a 65° C. for 16 h. The reaction was acidified by NH₄Cl solution to pH=4-5. The aqueous solution was extracted with a co-solvent (CHCl₃:i-PrOH=4:1), dried over Na₂SO₄ and filtered. The filtrate was concentrated in vacuo and the residue was purified by column chromatography on silica gel (CH₂Cl₂:MeOH=1:80-1:40) to give desired compound 36 (70 mg, 47%). ¹H NMR (300 MHz, CDCl₃) δ 11.84 (br, 2H), 8.97 (dd, J=1.8 and 4.2 Hz, 2H), 8.83 (dd, J=3.6 and 6.0 Hz, 2H), 8.16 (dd, J=1.5 and 8.1 Hz, 2H), 7.95 (d, J=8.1 Hz, 2H), 7.56-7.46 (m, 8H), 7.23 (br, 1H), 6.05 (br, 1H), 2.35-2.19 (m, 4H), 1.69-1.62 (m, 4H), 1.36 (m, 4H); ¹³C NMR (75 MHz, CDCl₃) δ 177.76, 170.92, 167.60, 148.19, 138.24, 137.61, 135.27, 134.44, 132.86, 127.15, 126.10, 126.00, 121.98, 120.95, 119.04, 116.29, 78.75, 36.47, 32.92, 27.70, 27.62, 24.29, 23.51. HR-MS Calcd. for (C₃₅H₃₃N₅O₆+H) 620.2509. found 620.2495.

### N¹-tert-Butoxy-N⁸-(4-(2-hydroxy-1,3-dioxo-1,3-bis(quinolin-8-ylamino)propan-2-yl)phenyl)octanediamide (37)

To a mixture of acid 36 (46 mg, 0.074 mmol), O-(tert-butyl)hydroxylamine hydrochloride (9.8 mg, 0.077 mmol) and triethylamine (10 μL, 0.074 mmol) in CH₂Cl₂ was added 1-ethyl-3-(3′-dimethylaminopropyl)carbodiimide (21.3 mg, 0.11 mmol) at room temperature. The reaction was quenched with MeOH after 15 h. After acidified by NH₄Cl solution to pH=4-5, the mixture was extracted with EtOAc, dried over Na₂SO₄ and filtered. The filtrate was concentrated in vacuo and the residue was purified by column chromatography on silica gel (CH₂Cl₂:MeOH=1:80-1:40) to give desired compound 37 (41 mg, 80%). ¹H NMR (300 MHz, CDCl₃) δ 11.83 (br, 2H), 8.91-7.28 (m, 18H), 6.04 (br, 1H), 2.23-1.21 (m, 21H); ¹³C NMR (75 MHz, CDCl₃) δ 172.75, 172.23, 168.81, 149.41, 139.49, 139.08, 136.49, 135.49, 134.14, 128.40, 127.34, 127.16, 123.16, 122.18, 120.18, 117.46, 82.11, 53.55, 37.55, 33.44, 30.10, 28.73, 26.66, 25.49. HR-MS Calcd. for (C₃₅H₃₃N₅O₆+H) 691.3251. found 691.3244.

### N¹-Hydroxy-N⁸-(4-(2-hydroxy-1,3-dioxo-1,3-bis(quinolin-8-ylamino)propan-2-yl)phenyl)octanediamide (38)

Compound 37 (41 mg, 0.059 mmol) was treated with trifluoroacetic acid (1 mL) in CH₂Cl₂ (1 mL) at room temperature for 72 h. After removing the volatiles, the residue was purified by column chromatography on silica gel (MeOH:CH₂Cl₂=1:40-1:15) to give desired compound 38 (24 mg, 64%). ¹H NMR (400 MHz, DMSO-d6) δ 11.61 (br, 2H), 10.29 (br, 1H), 9.94 (br, 1H), 9.00 (d, J=2.8 Hz, 2H), 8.76 (d, J=7.2 Hz, 2H), 8.62 (br, 1H), 8.46 (d, J=8.4 Hz, 2H), 7.90 (s, 1H), 7.76-7.60 (m, 10H), 2.27 (t, J=6.8 Hz, 2H), 1.92 (t, J=7.6 Hz, 2H), 1.55-1.47 (m, 4H), 1.24 (m, 4H); ¹³C NMR (75 MHz, DMSO-d6) δ 172.19, 169.92, 169.20, 150.31, 140.27, 138.95, 137.59, 135.14, 134.32, 128.73, 127.89, 127.55, 123.58, 123.30, 119.73, 116.89, 81.50, 37.15, 33.08, 29.22, 25.82. HR-MS Calcd. for (C₃₅H₃₄N₆O₆+H) 635.2618. found 635.2632.

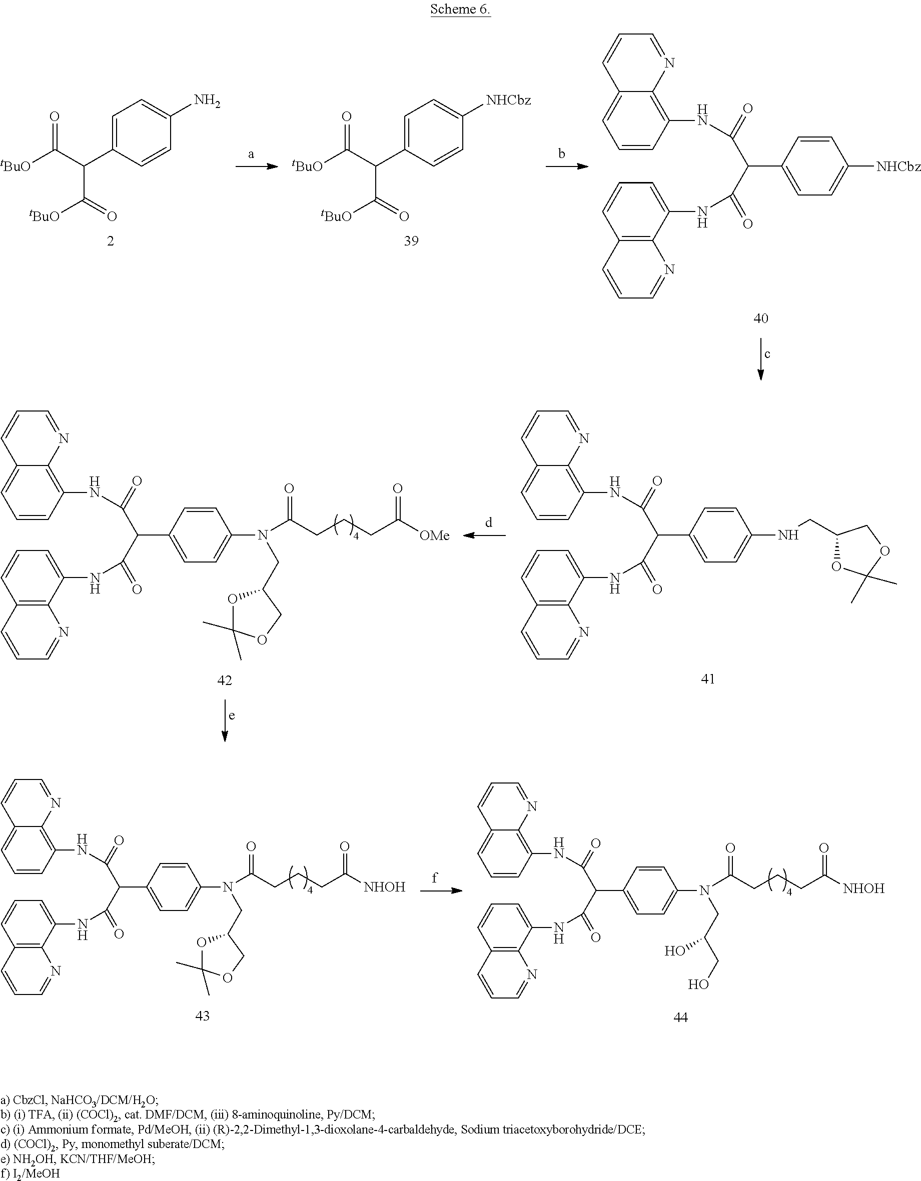

### Di-tert-butyl 2-(4-(benzyloxycarbonylamino)phenyl)malonate (39)

Compound 2 (100 mg, 0.33 mmol) was dissolved in a mixture solvent (CH₂Cl₂:H₂O=1 ml:1 ml) and the pH was adjusted to 8.0-9.0 by using NaHCO₃ aqueous solution. Benzylchloroformate (59 μL, 0.39 mmol) was then added. The reaction was kept at room temperature for 2.5 h and washed with H₂O. Organic layer was dried over Na₂SO₄ and filtered. The filtrate was concentrated in vacuo and the residue was purified by column chromatography on silica gel (hexanes:EtOAc=1:20-1:10) to give desired compound 39 (150 mg, 100%) ¹H NMR (400 MHz, CDCl₃) δ7.44-7.30 (m, 9H), 6.68 (br, 1H), 5.23 (s, 2H), 4.40 (s, 1H), 1.47 (s, 18H); HR-MS Calcd. for (C₂₅H₃₀NO₆+H) 634.2778. found 634.2793.

### Benzyl 4-(1,3-dioxo-1,3-bis(quinolin-8-ylamino)propan-2-yl)phenylcarbamate (40)

The title compound (140 mg, 74%) was prepared analogously to the procedure for compound 5 described above. ¹H NMR (400 MHz, DMSO-d6) δ11.02 (s, 2H), 9.82 (s, 1H), 8.94-8.93 (m, 2H), 8.68 (d, J=7.2 Hz, 2H), 8.42 (d, J=8.4 Hz, 2H), 7.73-7.33 (m, 15H), (br, 1H), 5.71 (s, 1H), 5.15 (s, 2H), 1.47 (s, 18H); HR-MS Calcd. for (C₃₅H₂₇N₅O₄+H) 582.2141. found 582.2155.

### 2-(4-((2,2-dimethyl-1,3-dioxolan-4-yl)methylamino)phenyl)-N¹,N³-di(quinolin-8-yl)malonamide (41)

A mixture of compound 40 (580 mg, 1.3 mmol) and (R)-2,2-dimethyl-1,3-dioxolane-4-carbaldehyde (169 mg, 1.3 mmol) in 1,2-dichloroethane was treated with sodium triacetoxyborohydride (407 mg, 1.4 mg) at room temperature overnight. Additional (R)-2,2-dimethyl-1,3-dioxolane-4-carbaldehyde (169 mg, 1.3 mmol) and sodium triacetoxyborohydride (407 mg, 1.4 mg) were added and the reaction was kept for 3 h. After quenching with NaHCO₃ solution to pH˜7.0, the reaction mixture was extracted with CH₂Cl₂. The organic layer was dried over Na₂SO₄ and filtered. The filtrate was concentrated in vacuo and the residue was purified by column chromatography on silica gel (MeOH:CH₂Cl₂=1:60-1:30) to give desired compound 41 (640 mg, 88%) ¹H NMR (400 MHz, CDCl₃) δ 10.92 (s, 2H), 8.87-8.84 (m, 4H), 8.16-8.13 (m, 2H), 7.60-7.42 (m, 8H), 6.69-6.66 (m, 2H), 4.86 (s, 1H), 4.37-4.31 (m, 1H), 4.10-4.05 (m, 2H), 3.77-3.72 (m, 1H), 3.32-3.16 (m, 2H), 1.44 (s, 3H), 1.36 (s, 3H); HR-MS Calcd. for (C₃₃H₃₁N₅O₄+H) 562.2473. found 562.2454.

### N¹-((2,2-dimethyl-1,3-dioxolan-4-yl)methyl)-N¹-(4-(1,3-dioxo-1,3-bis(quinolin-8-ylamino)propan-2-yl)phenyl)-N⁸-hydroxyoctanediamide (43)

Monomethyl suberate (200 mg, 1.05 mmol) in CH₂Cl₂ (1.5 mL) was treated with oxalyl chloride (0.1 ml, 1.16 mmol) in the presence of catalytic amount of DMF for 15 min. To this solution, pyridine (0.42 mL, 5.25 mmol) and compound 41 (590 mg, 1.05 mmol) in CH₂Cl₂ (5 mL) were added sequentially. After 20 min, the reaction was quenched with MeOH and poured into H₂O. The mixture was extracted with CH₂Cl₂ and dried over Na₂SO₄ and filtered. The filtrate was concentrated in vacuo and the residue was purified by column chromatography on silica gel (MeOH:CH₂C2=1:60-1:20) to give desired coupled compound 42 (350 mg), which was directly used for next step. Compound 42 (240 mg) was treated with hydroxylamine aqueous solution in the presence of catalytic amount of KCN in THF/MeOH solution for 36 h. After quenching with NH₄Cl solution, the aqueous phase was extracted and dried over Na₂SO₄ and filtered. The filtrate was concentrated in vacuo and the residue was purified by column chromatography on silica gel (MeOH:CH₂Cl₂=1:40-1:15) to give desired coupled compound 43 (130 mg, 25% from 41). ¹H NMR (400 MHz, DMSO-d6) δ 11.03 (s, 2H), 10.25 (s, 1H), 8.93 (m, 2H), 8.69 (d, J=7.6 Hz, 2H), 8.61 (s, 1H), 8.43 (d, J=8.0 Hz, 2H), 7.80-7.43 (m, 12H), 5.89 (s, 1H), 4.11 (m, 1H), 3.92 (m, 1H), 3.75 (m, 2H), 3.54 (m, 1H), 2.00 (m, 2H), 1.84 (m, 2H), 1.38 (m, 4H), 1.20 (s, 3H), 1.17 (s, 3H), 1.07 (m, 4H); HR-MS Calcd. for (C₄₁H₄₄N₆O₇+H) 733.3377. found 733.3350.

### N¹-(2,3-dihydroxypropyl)-N¹-(4-(1,3-dioxo-1,3-bis(quinolin-8-ylamino)propan-2-yl)phenyl)-N-hydroxyoctanediamide (44)

To a solution of compound 43 (105 mg, 0.14 mmol) in MeOH (10 mL) was added iodine (100 mg) and the reaction mixture was refluxed for 3 h. After being quenched with sodium thiosulfate solid, the mixture was concentrated in vacuo to dry. The residue was dissolved in MeOH and filtered. The filtrate was concentrated in vacuo and the residue was purified by column chromatography on a reverse phase C-18 silica gel (CH₃CN:H₂O=2:3) to give desired compound 44 (55 mg, 55%) ¹H NMR (400 MHz, MeOH-d4) δ 8.85-8.84 (m, 2H), 8.72-8.70 (m, 2H), 8.27-8.24 (m, 2H), 7.94-7.92 (m, 2H), 7.63-7.44 (m, 8H), 5.50 (s, 1H), 3.80 (m, 3H), 3.51-3.48 (m, 2H), 2.10 (t, J=7.2 Hz, 2H), 1.93 (t, J=7.2 Hz, 2H), 1.48-1.42 (m, 4H), 1.09 (m, 4H); HR-MS Calcd. for (C₃₈H₄₀N₆O₇+H) 693.3054. found 693.3037.

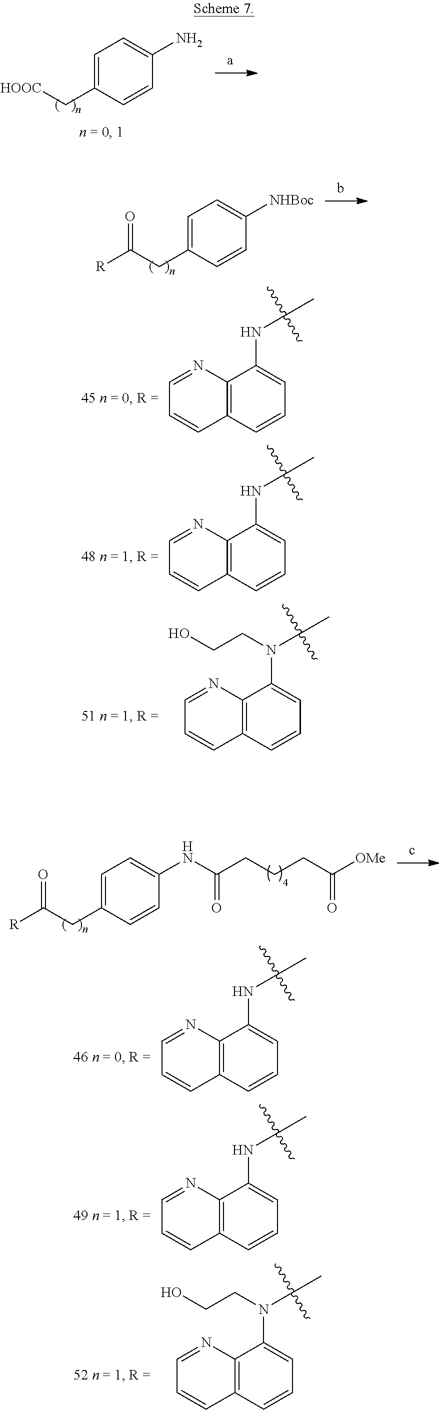

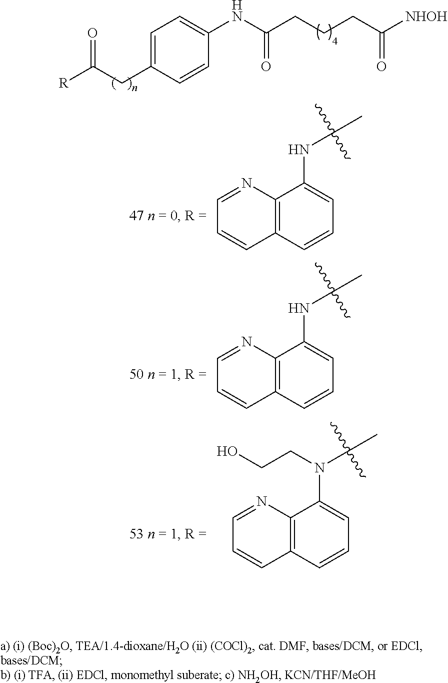

### tert-Butyl 4-(quinolin-8-ylcarbamoyl)phenylcarbamate (45)

A solution of 4-aminobenzoic acid (1.0 g, 7.3 mmol) and triethylamine (3.0 mL, 21.8 mmol) in 1,4-dioxane/H₂O was treated di-tert-butyl carbonate (2.5 mL, 10.9 mmol) at room temperature overnight. After removing the solvent in vacuo, the residue was dissolved in EtOAc and washed with 1M HCl solution. The organic phase was then extracted with 1M NaOH solution three times. The aqueous layer was then acidified by 1M HCl solution and the precipitation was collected and washed with H₂O to give 4-(tert-butoxycarbonylamino)benzoic acid (1.58 g, 91%). To a solution of 4-(tert-butoxycarbonylamino)benzoic acid (150 mg, 0.63 mmol) and pyridine (108 μL, 1.26 mmol) in CH₂Cl₂ was added oxalyl chloride (57 μL, 0.63 mmol) at 0° C. for 15 min. 8-Aminoquiniline (90 mg, 0.63 mmol) was then added to the mixture. After kept at room temperature for 1 h, then reaction was quenched with MeOH and poured into H₂O and extracted with CH₂Cl₂, dried over Na₂SO₄ and filtered. The filtrate was concentrated in vacuo and the residue was purified by column chromatography on silica gel (MeOH:CH₂Cl₂=1:40-1:30) to give compound 45 (150 mg, 65%) ¹H NMR (300 MHz, CDCl₃) δ10.72 (s, 1H), 8.95-8.87 (m, 2H), 8.21-8.04 (m, 4H), 7.63-7.47 (m, 4H), 6.82 (s, 1H), 1.55 (s, 9H); HR-MS Calcd. for (C₂₁H₂₁N₃O₃+H) 364.1661. found 364.1673.

### Methyl 8-oxo-8-(4-(quinolin-8-ylcarbamoyl)phenylamino)octanoate (46)

Compound 45 (180 mg, 0.50 mmol) was treated with trifluoroacetic acid (3 mL) at room temperature for 30 min. After removing the volatiles, the residue was dissolved in CH₂Cl₂ and triethylamine (70 μL, 0.50 mmol) was added followed by monomethyl suberate (94 μL, 0.5 mmol) and 1-ethyl-3-(3′-dimethylaminopropyl)carbodiimide (96 mg, 0.50 mmol). The reaction mixture was kept at room temperature for overnight and white precipitation appeared. The precipitation was collected and washed with EtOH and MeOH to give the target compound 46 (70 mg, 32%). ¹H NMR (400 MHz, DMSO-d6) δ 10.62 (s, 1H), 10.26 (s, 1H), 9.00 (dd, J=1.6 and 4.4 Hz, 1H), 8.75 (d, J=6.4 Hz, 1H), 8.49 (dd, J=1.6 and 8.4 Hz, 1H), 8.01 (d, J=8.8 Hz, 2H), 7.84 (d, J=8.4 Hz, 2H), 7.76-7.65 (m, 3H), 3.60 (s, 3H), 2.35 (dt, J=7.2 and 14.8 Hz, 4H), 1.62-1.54 (m, 4H), 1.33-1.32 (m, 4H); HR-MS Calcd. for (C₂₅H₂₇N₃O₄+H) 434.2080. found 434.2076.

### N¹-hydroxy-N⁸-(4-(quinolin-8-ylcarbamoyl)phenyl)octanediamide (47)

The title compound 47 (30 mg, 50%) was prepared analogously to the procedure for compound 6 described above. 1H NMR (400 MHz, DMSO-d6) δ 10.62 (s, 1H), 10.35 (s, 1H), 10.27 (s, 1H), 9.00-8.99 (m, 1H), 8.75 (d, J=6.4 Hz, 1H), 8.68 (s, 1H), 8.49-8.47 (m, 1H), 8.02 (d, J=8.8 Hz, 2H), 7.84 (d, J=8.8 Hz, 2H), 7.76-7.65 (m, 3H), 2.37 (t, J=7.2 Hz, 2H), 1.96 (t, J=7.2 Hz, 2H), 1.62-1.51 (m, 4H), 1.31-1.25 (m, 4H); HR-MS Calcd. for (C₂₄H₂₆N₄O₄+H) 435.2032. found 435.2021.

### tert-Butyl 4-(2-oxo-2-(quinolin-8-ylamino)ethyl)phenylcarbamate (48)

The title compound (220 mg, 63%) was prepared analogously to the procedure for compound 45 described above. ¹H NMR (300 MHz, CDCl₃) δ 9.93 (s, br, 1H), 8.78-8.73 (m, 2H), 8.16-8.13 (m, 1H), 7.56-7.36 (m, 7H), 6.51 (s, 1H), 3.86 (s, 2H), 1.54 (s, 9H); HR-MS Calcd. for (C₂₂H₂₃N₃O₃+H) 378.1818. found 378.1800.

### Methyl 8-oxo-8-(4-(4-(2-oxo-2(quinolin-8-ylamino)ethyl)phenylamino)octanoate (49)

The title compound (180 mg, 69%) was prepared analogously to the procedure for compound 46 described above. ¹H NMR (300 MHz, CDCl₃) δ 9.96 (s, br, 1H), 8.78-8.74 (m, 2H), 8.16 (d, J=8.4 Hz, 1H), 7.59-7.40 (m, 7H), 7.24 (s, br, 1H), 3.88 (s, 2H), 2.40-2.31 (m, 4H), 1.79-1.59 (m, 4H), 1.27 (m, 4H); HR-MS Calcd. for (C₂₆H₂₉N₃O₄+H) 448.2236. found 448.2223.

### N¹-hydroxy-N⁸-(4-(2-oxo-2-(quinolin-8-ylamino)ethyl)phenyl)octanediamide (50)

The title compound (45 mg, 28%) was prepared analogously to the procedure for compound 6 described above. ¹H NMR (400 MHz, DMSO-d6) δ 10.34 (s, 1H), 10.22 (s, 1H), 9.88 (s, 1H), 8.90 (dd, J=1.6 and 4.4 Hz, 1H), 8.68 (s, 1H), 8.62 (d, J=7.2 Hz, 1H), 8.41 (dd, J=1.2 and 8.0 Hz, 1H), 7.68-7.55 (m, 5H), 7.33 (d, J=8.4 Hz, 2H), 3.89 (s, 2H), 2.29 (t, J=7.2 Hz, 2H), 1.94 (t, J=7.2 Hz, 2H), 1.59-1.48 (m, 4H), 1.29 (m, 4H); HR-MS Calcd. for (C₂₅H₂₈N₄O₄+H) 449.2189. found 449.2181.

### tert-Butyl 4-(2-((2-hydroxyethyl)(quinolin-8-yl)amino)-2-oxoethyl)phenylcarbamate (51)

The title compound (100 mg, 27%) was prepared analogously to the procedure for compound 45 described above. ¹H NMR (300 MHz, CDCl₃) δ 8.94 (d, J=2.7 Hz, 1H), 8.34-8.31 (m, 1H), 7.95-7.93 (m, 1H), 7.62-7.51 (m, 3H), 7.36 (s, 1H), 7.13 (d, J=8.4 Hz, 2H), 6.77 (d, J=7.2 Hz, 2H), 6.39 (s, 1H), 4.80-4.72 (m, 1H), 4.04-3.97 (m, 1H), 3.50-3.32 (m, 2H), 3.25 (d, J=15.0, 1H), 3.09 (d, J=15.0, 1H), 1.53 (s, 9H); HR-MS Calcd. for (C₂₄H₂₇N₃O₄+H) 422.2080. found 422.2088.

### Methyl 8-(4-(2-((2-hydroxyethyl)(quinolin-8-yl)amino)-2-oxoethyl)phenylamino)-8-oxooctanoate (52)

The title compound (78 mg, 67%) was prepared analogously to the procedure for compound 46 described above. ¹H NMR (400 MHz, CDCl₃) δ 8.94 (dd, J=1.6 and 3.6 Hz, 1H), 8.32 (dd, J=1.6 and 8.0 Hz, 1H), 7.94 (dd, J=1.2 and 8.0 Hz, 1H), 7.85 (s, 1H), 7.63-7.54 (m, 4H), 7.28 (d, J=7.2 Hz, 2H), 6.77 (d, J=7.6 Hz, 2H), 4.81-4.75 (m, 1H), 3.98-3.93 (m, 1H), 3.67 (s, 3H), 3.48-3.34 (m, 2H), 3.22 (d, J=15.2, 1H), 3.11 (d, J=15.2, 1H), 2.32 (t, J=7.2 Hz, 4H), 1.72-1.62 (m, 4H), 1.38-1.36 (m, 4H); HR-MS Calcd. for (C₂₈H₃₃N₃O₅+H) 492.2498. found 492.2499.

### N¹-hydroxy-N⁸-(4-(2-((2-hydroxyethyl)(quinolin-8-yl)amino)-2-oxoethyl)phenyl)octanediamide (53)

The title compound (22 mg, 29%) was prepared analogously to the procedure for compound 6 described above. ¹H NMR (400 MHz, DMSO-d6) δ 10.34 (s, 1H), 9.76 (s, 1H), 8.98 (d, J=2.8 Hz, 1H), 8.67 (s, 1H), 8.50 (d, J=8.0 Hz, 1H), 7.79 (d, J=7.2 Hz, 1H), 7.71-7.63 (m, 3H), 7.37 (d, J=8.0 Hz, 2H), 6.79 (d, J=8.4 Hz, 2H), 4.69 (s, br, 1H), 4.11-4.06 (m, 1H), 3.50-3.38 (m, 3, 3H), 3.11 (d, J=15.6, 1H), 3.02 (d, J=15.2, 1H), 2.26 (t, J=7.6 Hz, 2H), 1.94 (t, J=7.2 Hz, 2H), 1.55-1.47 (m, 4H), 1.20 (m, 4H); HR-MS Calcd. for (C₂₇H₃₂N₄O₅+H) 493.2451. found 493.2467.

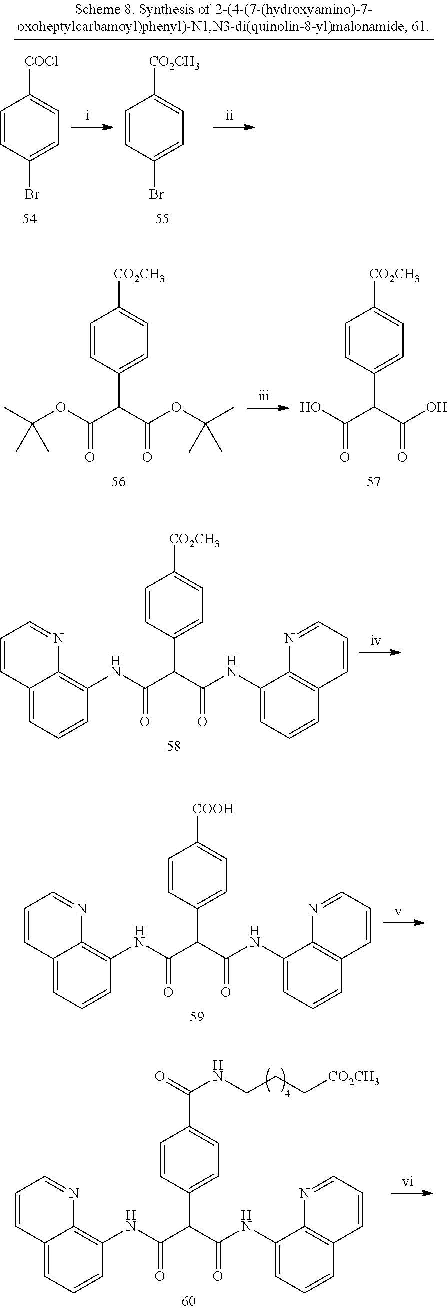

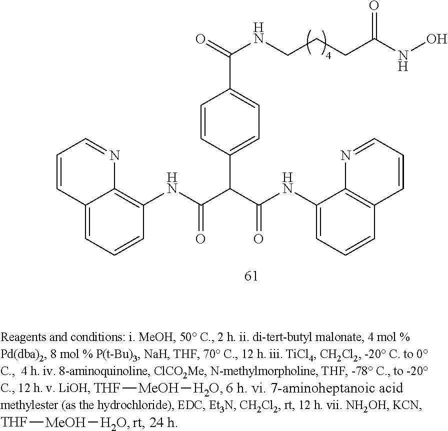

### Di-tert-butyl 2-(4-(methoxycarbonyl)phenyl)malonate, 56

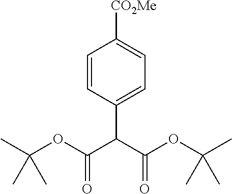 [SMILES: COC(=O)c1ccc(C(C(=O)OC(C)(C)C)C(=O)OC(C)(C)C)cc1]

To a Schlenk tube under argon, added NaH (270 mg, 11.25 mmol) followed by THF (5 mL). To this added di-tert-butylmalonate (2.43 g, 11.25 mmol) drop wise. When the gas evolution is over added 55 (2.2 g, 10.23 mmol) followed by Pd(dba)₂ (235 mg, 0.409 mmol) and P(t-Bu)₃ (1.64 mL 0.5 M soln in THF). After adding additional amount of THF (5 mL), the tube was thoroughly purged with argon, sealed and heated at 70° C. for 12 h. The cooled reaction mixture was filtered through Celite, washed with THF and concentrated. The residue was purified through flash column (silica gel 230-400 mesh) using 10% ethyl acetate in hexanes as the eluent. Compound 56 was obtained as a white solid (3.385 g, 94%).

Mp=75-77° C. ¹H NMR (300 MHz, CDCl₃): δ=1.48 (s, 18H), 3.94 (s, 3H), 4.51 (s, 1H), 7.49 (d, 2H, J=6.3 Hz), 8.05 (d, 2H, J=6.3 Hz). ¹³C NMR (75 MHz, CDCl₃): δ=28.2, 52.5, 60.4, 82.7, 129.8, 130.0, 138.9, 167.2.

### 2-(4-(Methoxycarbonyl)phenyl)malonic acid, 57

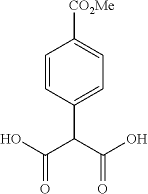 [SMILES: COC(=O)c1ccc(C(C(=O)O)C(=O)O)cc1]

To compound 56 (2 g, 5.71 mmol) in CH₂Cl₂ (40 mL) at −20° C., added TiCl₄ (17.1 mL 1 M soln in CH₂Cl₂) drop wise. After the addition the solution was slowly brought to 0° C. and kept at that temperature for 4 h. The reaction mixture was cooled down to −20° C. again and quenched by the addition of water. CH₂Cl₂ was removed and the residue was worked up with ethyl acetate. The organic layer was dried with anhyd Na₂SO₄ and the solvent was removed under reduced pressure. The residue was triturated with 1:1 mixture of diethyl ether-hexanes and dried leaving compound 57 as a white solid (1.25 g, 92%).

¹H NMR (300 MHz, DMSO): δ=3.79 (s, 3H), 4.54 (s, 1H), 7.43 (d, 2H, J=8.1 Hz), 7.90 (d, 2H, J=8.4 Hz). ¹³C NMR (75 MHz, DMSO): δ=51.9, 57.9, 129.4, 138.7, 166.6, 169.6.

### Methyl 4-(1,3-dioxo-1,3-bis(quinolin-8-ylamino)propan-2-yl)benzoate, 58

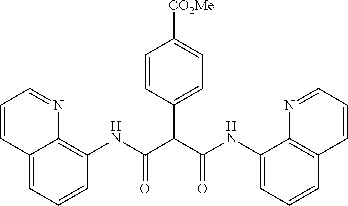 [SMILES: COC(=O)c1ccc(C(C(=O)Nc2cccc3cccnc23)C(=O)Nc2cccc3cccnc23)cc1]

Compound 57 (200 mg, 0.84 mmol) was dissolved in THF (20 mL) and cooled down to −78° C. To the cold solution methyl chloroformate (159 mg, 1.68 mmol) was added followed by N-methylmorpholine (170 mg, 1.68 mmol). The solution was allowed to stir for five minutes and then a solution of 8-aminoquinoline (242 mg, 1.68 mmol) and N-methylmorpholine (170 mg, 1.68 mmol) in THF (5 mL) was added drop wise. The reaction mixture was kept at −78° C. for 2 h and slowly warmed to −20° C., and kept at that temperature overnight (in the freezer). The solution was then warmed to rt and filtered through Celite, washed with THF. The volatiles were removed under vacuum and the residue purified through column chromatography (silica gel 230-400 mesh) using 30-50% ethyl acetate in hexanes followed by CH₂Cl₂ as the eluents. The product was recrystallized from CH₂Cl₂-hexanes to yield 58 as a pale yellow crystalline solid (207 mg, 50%).

¹H NMR (400 MHz, CDCl₃): δ=3.92 (s, 3H), 5.32 (s, 1H), 7.55 (dd, 2H, J=4.4 Hz, J₂=8.4 Hz), 7.59-7.64 (m, 4H), 7.94 (d, 2H, J=8.4 Hz), 8.12 (d, 2H, J=8.4 Hz), 8.27 (dd, 2H, J₁=1.6 Hz, J₂=8.4 Hz), 8.89-8.91 (m, 4H), 11.12 (s, 2H). ¹³C NMR (75 MHz, CDCl₃): δ=52.6, 63.0, 117.6, 122.1, 122.8, 127.5, 128.3, 129.0, 130.5, 130.8, 134.5, 136.6, 139.2, 140.2, 149.0, 166.8, 167.0.

### 4-(1,3-dioxo-1,3-bis(quinolin-8-ylamino)propan-2-yl)benzoic acid, 59

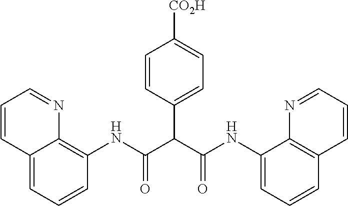 [SMILES: O=C(O)c1ccc(C(C(=O)Nc2cccc3cccnc23)C(=O)Nc2cccc3cccnc23)cc1]

To compound 58 (300 mg, 0.63 mmol) in THF (5 mL) under argon was added a solution of LiOH.H₂O in water (2 mL). The reaction mixture was allowed to stir at rt for 12 h. THF was removed under vacuum and the mixture was acidified with 2N HCl. The precipitate was collected by filtration, washed with water and dried to obtain compound 59 as a white solid (219 mg, 75%).

¹H NMR (300 MHz, DMSO): δ=5.02 (s, 1H), 7.38-7.48 (m, 6H), 7.79 (d, 2H, J=8.4 Hz), 8.03-8.11 (m, 4H), 8.74-8.81 (m, 4H), 10.93 (s, 2H). ¹³C NMR (75 MHz, DMSO): δ=60.5, 63.0, 117.0, 118.3, 123.1, 123.7, 127.8, 128.7, 129.8, 130.3, 131.7, 134.9, 137.6, 139.2, 150.0, 150.3, 167.8, 168.6. LRMS Calcd for C₂₈H₂₀N₄O₄: 477.15 Found: 476.97.

### Methyl 7-(4-(1,3-dioxo-1,3-bis(quinolin-8-ylamino)propan-2-yl)benzamido)heptanoate, 60

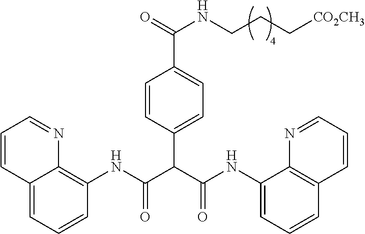

To compound 59 (200 mg, 0.42 mmol) in CH₂Cl₂ (20 mL) at rt added 7-aminoheptanoic acid methyl ester (as the hydrochloride, 90 mg, 0.46 mmol), Et₃N (47 mg, 0.46 mmol) and EDC (89 mg, 0.46 mmol). The mixture was allowed to stir at rt for 12 h. Water was added and the mixture was worked up with CH₂Cl₂. Combined organic extracts were dried with anhyd Na₂SO₄, solvent removed under vacuum and the residue was purified by column chromatography (silica gel 230-400 mesh, 80% ethyl acetate in hexanes as the eluent) to afford 70 as a white solid (202 mg, 78%).

Mp=178-180° C. ¹H NMR (300 MHz, CDCl₃): δ=1.34-1.36 (m, 4H), 1.59-1.62 (m, 4H), 2.29 (t, 2H, J=5.7 Hz), 3.42 (m, 2H), 3.64 (s, 3H), 5.03 (s, 1H), 6.14 (s, 1H), 7.43-7.47 (m, 2H), 7.53 (d, 4H, J=3.3 Hz), 7.84 (dd, 4H, J₁=6.3 Hz, J₂=15.3 Hz), 8.15 (d, 2H, J=6 Hz), 8.82-8.86 (m, 4H), 10.98 (s, 2H). ¹³C NMR (75 MHz, CDCl₃): δ=24.7, 26.5, 28.7, 29.4, 33.9, 40.0, 51.5, 62.3, 117.1, 121.7, 122.4, 127.1, 127.9, 128.7, 134.1, 134.8, 136.2, 138.0, 138.7, 148.7, 166.6, 167.0, 174.2. HRMS (FAB+) Calcd for C₃₆H₃₆O₅N₅=618.2638. Found=618.2712.

### 2-(4-(7-(hydroxyamino)-7-oxoheptylcarbamoyl)phenyl)-N1,N3-di(quinolin-8-yl)malonamide, 61

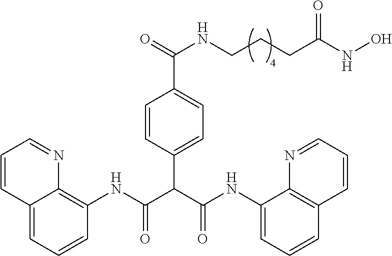

Compound 60 (150 mg, 0.24 mmol) was dissolved in THF-MeOH (4 mL, 3:1) and to this solution at rt under argon, added 50% aq. hydroxylamine solution (2 mL) followed by KCN (1.6 mg, 0.024 mmol). The reaction mixture was allowed to stir at rt for 24 h and then acidified to pH 6 with 2N HCl. The precipitate was filtered and washed thoroughly with water. It was then suspended in methanol and precipitated with ether. Filtered again and dried to obtain 61 as a white powder in quantitative yield (150 mg).

Mp=212-215° C. ¹H NMR (400 MHz, DMSO): δ=1.14-1.36 (m, 4H), 1.46-1.49 (m, 4H), 1.90-1.94 (m, 2H), 3.21-3.23 (m, 2H), 5.87 (s, 1H), 7.58-7.66 (m, 4H), 7.71-7.78 (m, 4H), 7.86-7.88 (m, 2H), 8.41-8.43 (m, 3H), 8.62-8.67 (m, 3H), 8.92-8.94 (m, 2H), 10.30 (s, 1H), 11.10 (s, 2H). ¹³C NMR (75 MHz, DMSO): S=25.6, 26.7, 28.8, 29.5, 32.7, 60.0, 117.7, 122.7, 123.2, 127.4, 128.1, 128.4, 129.0, 134.6, 134.8, 137.1, 138.9, 149.7, 166.3, 167.6, 169.6, HRMS (FAB+) Calcd for C₃₅H₃₄O₅N₆=619.2591. Found=619.2691.

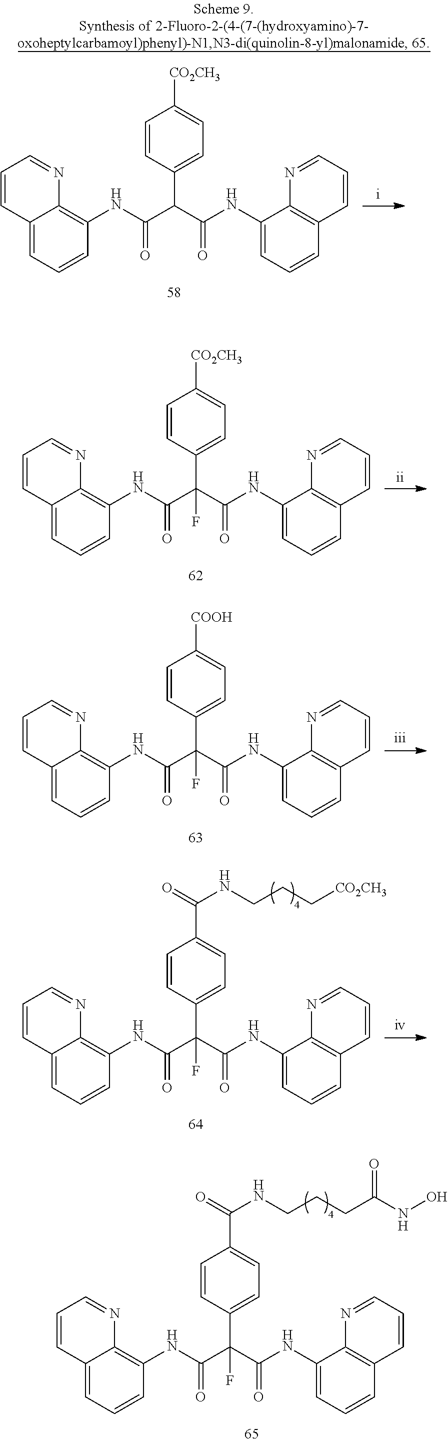

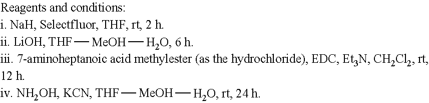

### Methyl 4-(2-fluoro-1,3-dioxo-1,3-bis(quinolin-8-ylamino)propan-2-yl)benzoate, 62

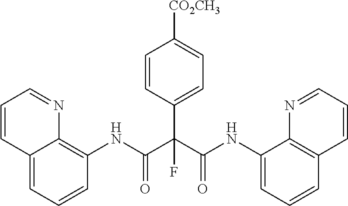 [SMILES: COC(=O)c1ccc(C(F)(C(=O)Nc2cccc3cccnc23)C(=O)Nc2cccc3cccnc23)cc1]

To compound 58 (350 mg, 0.71 mmol) in THF (20 mL) under argon, added NaH (19 mg, 0.79 mmol) and the mixture was allowed to stir for five minutes. Selectfluor (280 mg, 0.79 mmol) was then added and the reaction mixture was stirred for 4 h at rt. Solvent removed, added water and the mixture was worked up with CH₂Cl₂. The organic layer was dried with anhyd Na₂SO₄, solvent removed and the residue purified by flash column chromatography (silica gel 230-400 mesh, 30-50% ethyl acetate in hexanes as the eluent) to afford 62 as a light brown solid (296 mg, 82%).

¹H NMR (400 MHz, CDCl₃): δ=3.89 (s, 3H), 7.46-7.56 (m, 6H), 8.10-8.18 (m, 6H), 8.83 (dd, 2H, J₁=2 Hz, J₂=6.8 Hz), 8.91 (dd, 2H, J₂=2 Hz, J₂=4.2 Hz), 11.41 (s, 2H). ¹³C NMR (75 MHz, CDCl₃): δ=52.1, 117.1, 121.7, 122.8, 125.5, 125.7, 126.8, 127.7, 129.8, 133.1, 136.0, 138.6, 148.7, 163.1, 163.4, 166.2. LRMS Calcd for C₂₉H₂₁FN₄O₄=509.15. Found=508.73

### Methyl 7-(4-(2-fluoro-1,3-dioxo-1,3-bis(quinolin-8-ylamino)propan-2-yl)benzamido)heptanoate, 64

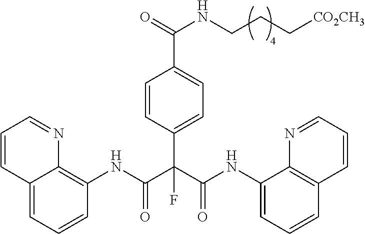

To compound 62 (200 mg, 0.41 mmol) in THF-MeOH (6 mL, 2:1) was added 52 mg LiOH.H₂O in water (2 mL). The mixture was allowed to stir under argon overnight. Volatiles were removed under vacuum, diluted with water and neutralized by 2N HCl. Worked up the solution with ethyl acetate, dried with anhyd Na₂SO₄, and concentrated. The crude product obtained was used for the next step without further purification.

To the crude 63 in CH₂Cl₂, added 7-aminoheptanoic acid methyl ester (as the hydrochloride 80 mg, 0.41 mmol), followed by Et₃N (41 mg, 0.41 mmol) and EDC (79 mg, 0.41 mmol). The mixture was allowed to stir overnight at rt. Water was added and the reaction was worked up with CH₂Cl₂. The organic layer was dried with anhyd Na₂SO₄, and the solvent was removed under reduced pressure. The residue was purified by flash column (silica gel 230-400 mesh, 60% ethyl acetate in hexanes as the eluent) to yield compound 64 as viscous oil (150 mg, 58%).

¹H NMR (400 MHz, CDCl₃): δ=1.33-1.34 (m, 4H), 1.56-1.61 (m, 4H), 2.25-2.29 (m, 2H), 3.38-3.43 (m, 2H), 3.63 (s, 3H), 6.15 (s, 1H), 7.46-7.49 (m, 2H), 7.54-7.56 (m, 4H), 7.82 (d, 2H, J=8 Hz), 8.07 (d, 2H, J=8.4 Hz), 8.15-8.17 (m, 2H), 8.82 (dd, 2H, J₁=2 Hz, J₂=6.8 Hz), 8.89-8.90 (m, 2H), 11.39 (s, 2H). ¹³C NMR (75 MHz, CDCl₃): δ=24.9, 26.7, 28.8, 29.5, 34.1, 40.2, 51.6, 117.4, 122.1, 123.2, 126.1, 126.2, 127.2, 127.6, 127.7, 128.1, 133.5, 136.4, 139.0, 149.0, 163.6, 163.9, 166.9, 174.3. LRMS Calcd for C₃₆H₃₄FO₅N₅=636.25. Found=636.30.

### 2-Fluoro-2-(4-(7-(hydroxyamino)-7-oxoheptylcarbamoyl)phenyl)-N1,N3-di(quinolin-8-yl)malonamide, 65

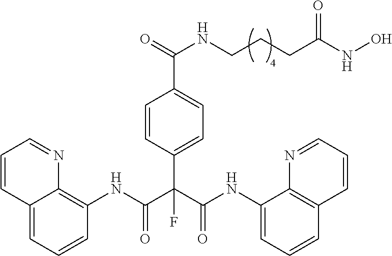

To compound 64 (100 mg, 0.16 mmol) in THF-MeOH (4 mL, 3:1) added hydroxylamine (2 mL, 50% aq. soln.) followed by KCN (1 mg, 0.016 mmol) and the mixture was allowed to stir at rt for 24 h. The reaction mixture was acidified with 2N HCl and the precipitate was filtered, washed with water and dried. The crude product was purified through flash column (silica gel 230-400 mesh, 5-10% MeOH in CH₂Cl₂ as the eluent) to yield 65 as a brown solid (66 mg, 66%).

Mp: 120-122° C. ¹H NMR (400 MHz, CDCl₃): δ=1.12-1.16 (m, 4H), 1.33-1.37 (m, 4H), 1.78-1.82 (m, 2H), 3.05-3.12 (m, 2H), 7.50-7.83 (m, 10H), 8.07 (s, 1H), 8.29-8.34 (m, 2H), 8.55 (d, 2H, J=8 Hz), 8.88-8.89 (m, 2H), 10.18 (s, 1H), 11.15 (s, 1H), 11.52 (s, 2H). ¹³C NMR (75 MHz, CDCl₃): δ=26.0, 27.0, 29.2, 29.8, 33.1, 117.0, 123.3, 123.7, 126.7, 127.0, 127.8, 128.1, 128.7, 133.5, 134.2, 135.7, 137.6, 138.9, 143.2, 150.3, 166.6, 168.7, 170.0. LRMS Calcd for C₃₅H₃₃FO₅N₆=637.25. Found=637.18.

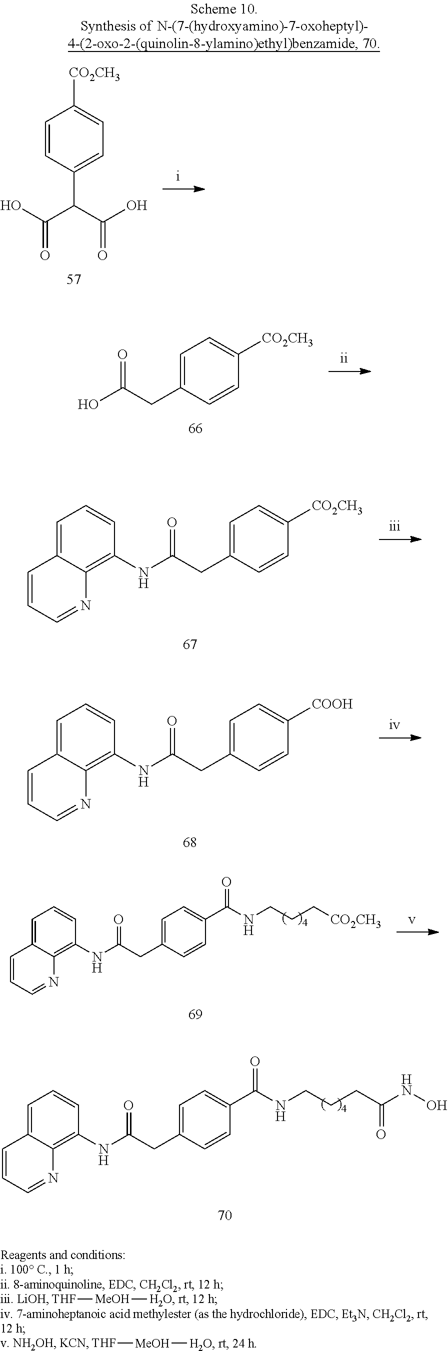

### Methyl 4-(2-oxo-2-(quinolin-8-ylamino)ethyl)benzoate, 67

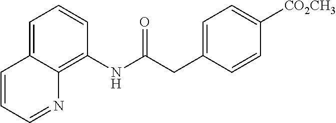 [SMILES: COC(=O)c1ccc(CC(=O)Nc2cccc3cccnc23)cc1]

Compound 57 (300 mg, 1.26 mmol) was heated at 100° C. for 1 h. The reaction mixture was dissolved in methanol and passed through a short silica gel column. To the crude 66 (240 mg, 1.24 mmol) in CH₂Cl₂ (20 mL) was added 8-aminoquinoline (196 mg, 1.36 mmol) and EDC (261 mg, 1.36 mmol) and the mixture was allowed to stir at rt for 12 h. Water was added and the mixture was worked up with CH₂Cl₂. The organic layer was dried with anhyd Na₂SO₄ and the solvent removed under reduced pressure. The residue was purified by flash column (silica gel 230-400 mesh, 30% ethyl acetate in hexanes as the eluent) to yield 67 as a colorless crystalline solid (342 mg, 85%).

¹H NMR (400 MHz, CDCl₃): δ=3.91 (s, 3H), 3.96 (s, 2H), 7.43-7.46 (m, 1H), 7.51-7.53 (m, 4H), 8.06 (d, 2H, J=8.4 Hz), 8.18-8.20 (m, 1H), 8.72-8.76 (m, 2H), 10.00 (s, 1H). ¹³C NMR (75 MHz, CDCl₃): δ=45.4, 52.4, 116.9, 121.9, 122.1, 127.6, 128.1, 129.4, 129.8, 130.4, 134.4, 136.8, 138.4, 140.2, 148.4, 167.1, 168.8. LRMS Calcd for C₁₉H₁₆N₂O₃: 321.11 Found: 321.07.

### 4-(2-Oxo-2-(quinolin-8-ylamino)ethyl)benzoic acid, 68

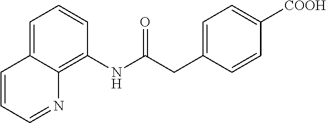 [SMILES: O=C(Cc1ccc(C(=O)O)cc1)Nc1cccc2cccnc12]

To compound 67 (300 mg, 0.94 mmol) in THF-MeOH (6 mL, 2:1) added LiOH.H₂O (118 mg, 2.81 mmol) in water (2 mL). Allowed to stir overnight at rt. Diluted with water and neutralized with 2N HCl. Worked up with ethyl acetate and the solvent removed under reduced pressure. The residue was triturated with CH₂Cl₂-hexanes and dried to yield 68 as a white solid (253 mg, 88%).

¹H NMR (400 MHz, CDCl₃): δ=4.08 (s, 2H), 7.53-7.77 (m, 6H), 7.94 (d, 2H, J=8 Hz), 8.42 (d, 1H, J=8.4 Hz), 8.61 (d, 1H, J=7.6 Hz), 8.93-8.94 (m, 1H), 10.37 (s, 1H). ¹³C NMR (75 MHz, CDCl₃): δ=43.3, 116.7, 122.0, 126.8, 127.8, 129.4, 129.5, 134.4, 136.5, 138.1, 140.8, 148.8, 167.3, 169.1. LRMS Calcd for C₁₈H₁₄N₂O₃: 307.10 Found: 307.06.

### Methyl 7-(4-(2-oxo-2-(quinolin-8-ylamino)ethyl)benzamido)heptanoate, 69

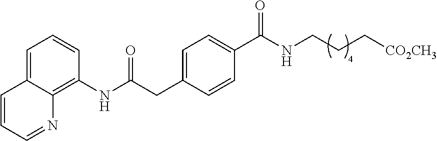

Compound 68 (200 mg, 0.65 mmol) was dissolved in CH₂Cl₂ (20 mL) under argon. To this added 7-aminoheptanoic acid methyl ester (as the hydrochloride, 140 mg, 0.72 mmol), Et₃N (73 mg, 0.72 mmol) and EDC (138 mg, 0.72 mmol) and the mixture was allowed to stir overnight at rt. Added water and worked up with CH₂Cl₂, the residue purified through flash column (silica gel 230-400 mesh, 80% ethyl acetate in hexanes) to obtain 69 as white solid (238 mg, 82%).

¹H NMR (400 MHz, CDCl₃): δ=1.35-1.37 (m, 4H), 1.58-1.64 (m, 4H), 2.29 (t, 2H, J=7.2 Hz), 3.43 (q, 2H, J=6.8 Hz), 3.64 (s, 3H), 3.92 (s, 2H), 6.18 (s, 1H), 7.41-7.51 (m, 5H), 7.77 (d, 2H, J=8 Hz), 8.15 (d, 1H, J=8 Hz), 8.71-8.73 (m, 2H), 9.95 (s, 1H). ¹³C NMR (75 MHz, CDCl₃): δ=24.7, 26.5, 28.6, 33.8, 39.9, 44.7, 51.3, 116.3, 121.6, 121.8, 127.0, 127.6, 127.7, 129.4, 133.7, 134.0, 136.2, 137.9, 138.1, 148.2, 167.3, 168.9, 174.1 LRMS Calcd for C₂₆H₂₉N₃O₄: 448.21 Found: 447.90.

### N-(7-(Hydroxyamino)-7-oxoheptyl)-4-(2-oxo-2-(quinolin-8-ylamino)ethyl)benzamide, 70

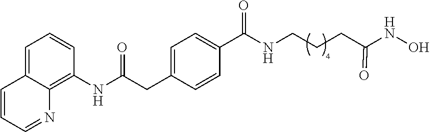

Compound 69 (200 mg, 0.45 mmol) was dissolved in THF-MeOH (4 mL, 3:1) and to this added hydroxylamine (2 mL, 50% aq. soln.) followed by KCN (2.9 mg, 0.045 mmol) and the solution was allowed to stir for 24 h at rt. Neutralized with 2N HCl and worked up with 1:1 ethyl acetate—THF mixture containing 5% isopropanol. The organic layer was dried with anhyd Na₂SO₄, and the solvent removed under reduced pressure. The residue was purified through flash column (silica gel 230-400 mesh, 4-10% methanol in CH₂Cl₂ as the eluent) to obtain 70 as viscous oil (121 mg, 60%).

¹H NMR (400 MHz, DMSO): δ=1.22-1.27 (m, 4H), 1.46-1.51 (m, 4H), 1.90-1.94 (m, 2H), 3.22 (dd, 2H, J₁=6.8 Hz, J₂=12.8 Hz), 4.02 (s, 2H), 7.48 (d, 2H, J=8.4 Hz), 7.55 (t, 1H, J=8 Hz), 7.61-7.67 (m, 2H), 7.81 (d, 2H, J=8.4 Hz), 8.40 (dd, 2H, J₁=1.6 Hz, J₂=8.4 Hz), 8.59 (d, 1H, J=7.8 Hz), 8.66 (s, 1H), 8.91 (dd, 1H, J₁=1.6 Hz, J₂=4 Hz), 10.32 (s, 2H). ¹³C NMR (75 MHz, CDCl₃): δ=26.0, 27.2, 29.3, 30.0, 33.2, 44.2, 117.7, 123.0, 123.1, 127.9, 128.2, 128.8, 130.2, 134.2, 135.4, 137.5, 139.1, 139.8, 149.8, 166.9, 170.2, 170.3 HRMS Calcd for C₂₅H₂₈N₄O₄: 449.2111 Found: 449.2192.

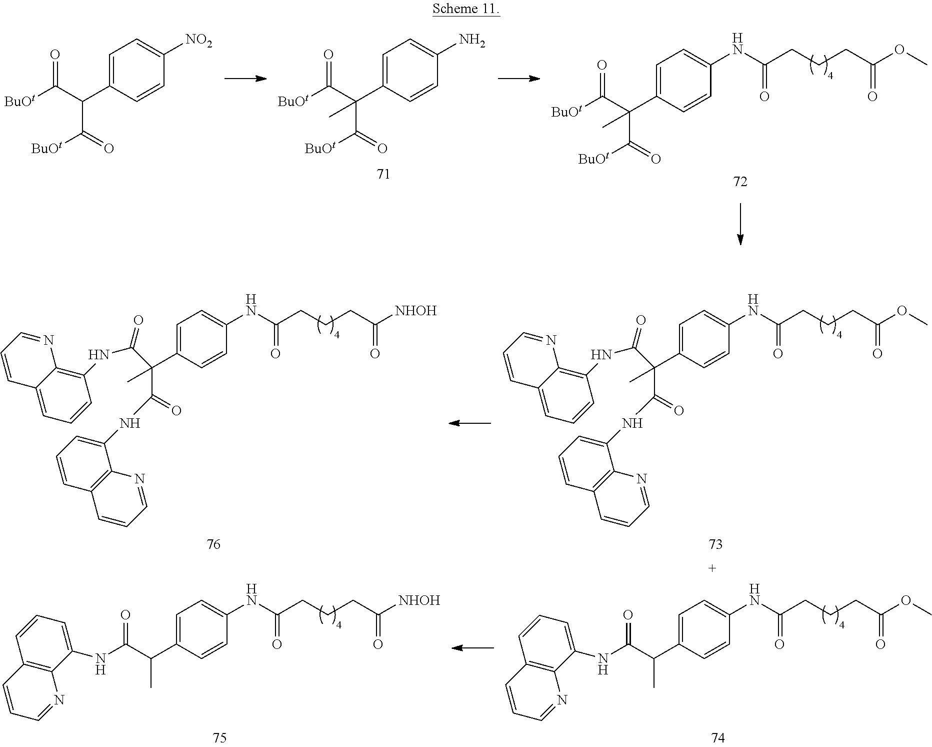

### Di-tert-butyl 2-(4-aminophenyl)-2-methylmalonate (71)

To a THF (10 mL) solution of di-tert-butyl 2-(4-nitrophenyl)malonate (700 mg, 2.1 mmol) was added NaH (91.4 mg, 2.3 mmol) 60% in mineral oil at 0° C. After 15 min, MeI (142 μl, 2.3 mmol) was added. The reaction mixture warmed up to RT. After 3 h, sat. NH₄Cl aq. solution was added to quench the reaction. The resulting solution was extracted with EtOAc (30 ml×3). The organic layers were combined, dried over Na₂SO₄ and concentrated under vacuum. The residue was dissolved in EtOH (10 mL) and treated with ammonium formate (1.32 g, 21 mmol) and Pd/C (10 wt %, 200 mg). The suspension was refluxed for 30 min and quenched with celite. After filtration, the filtrate was concentrated and dissolved in EtOAc (50 mL) and washed with H₂O (10 mL), brine (10 mL), dried over Na₂SO₄ and filtered. The filtrate was concentrated in vacuo and the residue was purified by column chromatography on a silica gel (Hexane:EtOAc=10/1-1/1) to give aniline 71 (546.7 mg, 2-step yield 82%) as yellow solid. ¹H NMR (300 MHz, CDCl₃): δ 7.20 (d, 2H, J=8.7 Hz), 6.64 (d, 2H, J=8.7 Hz), 3.63 (sb, 2H), 1.73 (s, 3H), 1.46 (s, 18H)); ¹³C NMR (300 MHz, CDCl₃) δ169.6, 147.2, 130.5, 130.4, 115.6, 82.4, 60.2, 28.7, 22.1. HR-MS Calcd. for C₁₈H₂₇NO₄ 321.4176. found 321.4184.

### Di-tert-butyl 2-(4-(8-methoxy-8-oxooctanamido)phenyl)-2-methylmalonate (72)

At 0° C., to a solution of 71 (546.7 mg, 1.70 mmol) and monomethyl suberate (0.34 mL, 1.87 mmol) in anhydrous CH₂Cl₂ (10 mL) was added 1-ethyl-3-(3′-dimethylaminopropyl)carbodiimide (358.9 mg, 1.87 mmol). The reaction mixture was allowed to warm up to room temperature and stirred overnight. The solution was washed with ice-cooled 1N NaOH (aq.) followed by H₂O, brine, dried over Na₂SO₄. The filtrate was concentrated in vacuo and the residue was purified by column chromatography on a silica gel (Hexane:EtOAc=10/1-1/1) to give amide 72 (712.0 mg, 85.2%). ¹H NMR (300 MHz, CDCl₃): δ 7.49 (d, 2H, J=8.7 Hz), 7.36 (d, 2H, J=8.7 Hz), 7.16 (s, 1H), 3.67 (s, 3H), 2.36-2.29 (m, 4H), 1.76-1.62 (m, 7H), 1.50-1.37 (m, 22H); ¹³C NMR (300 MHz, CDCl₃) 179.8, 173.1, 169.6, 137.3, 136.0, 129.9, 122.0, 82.4, 60.2, 51.9, 38.3, 33.6, 28.7, 28.3, 25.6, 25.0. HR-MS Calcd. for C₂₇H₄₁NO₇ 491.6235. found 491.6254.

### Methyl 8-(4-(2-methyl-1,3-dioxo-1,3-bis(quinolin-8-ylamino)propan-2-yl)phenylamino)-8-oxooctanoate (73) and Methyl 8-oxo-8-(4-(1-oxo-1-(quinolin-8-ylamino)propan-2-yl)phenylamino)octanoate (74)

Compound 72 (700 mg, 1.4 mmol) in CH₂Cl₂ (4 mL) was treated with trifluoroacetic acid (2 mL, 26 mmol) at room temperature for 24 h. After removing the volatile, the white solid was suspended in anhydrous CH₂Cl₂ (8 mL). The suspension was treated with oxalyl chloride (0.26 mL, 2.9 mmol) followed by DMF (0.11 mL, 1.4 mmol) at −30° C. to −15° C. for 30 min. The resulting solution was re-cooled to −60° C. and pyridine (0.51 mL, 6.3 mmol) was added followed by 8-aminoquinoline (413 mg, 2.8 mmol). The reaction mixture was allowed to warm up to −30° C. to −20° C. for 30 min before quenching with MeOH (1 mL) at −60° C. The solution was diluted with EtOAc (200 mL) and washed thoroughly with NH₄Cl (sat. aq.), dried over Na₂SO₄ and filtered. The filtrate was concentrated in vacuo and the residue was purified by column chromatography on a silica gel (CH₂Cl₂:MeOH=1:40-1/20) to give di-quinoline derivative 73 (405 mg, 45% from 72) and mono-quinoline derivative 74 (66 mg, 10% from 72). Compound 73: ¹H NMR (300 MHz, CDCl₃): δ 11.11 (s, 2H), 8.88 (d, 2H, J=6.6 Hz), 8.71 (d, 2H, J=3.9 Hz), 8.11 (d, 2H, J=8.1 Hz), 7.60-7.30 (m, 11H), 3.65 (s, 3H), 2.35-2.25 (m, 7H), 1.80-1.55 (m, 4H), 1.45-1.25 (m, 4H); ¹³C NMR (300 MHz, CDCl₃): δ 174.4, 171.6, 171.2, 148.7, 139.1, 138.0, 136.6, 136.2, 134.5, 128.2, 128.0, 127.3, 122.2, 121.7, 120.5, 117.0, 61.6, 51.6, 37.5, 34.0, 28.8, 25.4, 24.8, 24.2. HRMS-FAB (M+1) calcd for C₃₇H₃₈N₅O₅ 632.2873. found 632.2890.

Compound 74: ¹H NMR (300 MHz, CDCl₃): δ 9.90 (s, 1H), 8.75-8.70 (m, 2H), 8.09 (d, 1H, J=8.4 Hz), 7.55-7.30 (m, 8H), 3.89 (q, 1H, J=7.2 Hz, J=14.1 Hz), 3.64 (s, 3H), 2.35-2.22 (m, 4H), 1.78-1.50 (m, 7H), 1.41-1.24 (m, 4H); ¹³C NMR (300 MHz, CDCl₃): δ 174.4, 172.9, 171.4, 148.3, 138.6, 137.4, 136.9, 136.3, 134.6, 128.2, 128.0, 127.4, 121.7, 121.6, 120.4, 116.4, 51.6, 48.2, 37.6, 34.0, 29.0, 25.4, 24.8, 18.8. HRMS-FAB (M+1) calcd for C₂₇H₃₂N₃O₄ 462.2393. found 462.2401.

### N¹-hydroxy-N⁸-(4-(1-oxo-1-(quinolin-8-ylamino)propan-2-yl)phenyl)octanediamide (75)

A suspension of ester 74 (60 mg, 0.130 mmol), hydroxylamine (50% solution in water, 0.4 mL) and catalytic amount of KCN (0.3 mg) in a co-solvent (MeOH:THF=2 mL:2 mL) was stirred at 35° C. to 40° C. for 24 h. After removing the solvent, the residue was treated with NH₄Cl (sat. aq.) to pH=4-5. The mixture was extracted with a co-solvent (CHCl₃:i-PrOH=4:1), dried over Na₂SO₄ and filtered. The filtrate was concentrated in vacuo and the residue was purified by column chromatography on a silica gel (CH₂Cl₂:MeOH=1:40-1/10) to give target hydroxamic acid 76 (25 mg, 42%). ¹H NMR (300 MHz, CD₃OD): δ 8.78-8.76 (m, 1H), 8.62-8.60 (m, 1H), 8.28-8.25 (m, 1H), 7.61-7.41 (m, 7H), 4.05 (q, 1H, J=6.9 Hz, J=14.1 Hz), 2.36 (t, 2H, J=7.5 Hz), 2.08 (t, 2H, J=7.5 Hz), 1.75-1.55 (m, 7H), 1.45-1.27 (m, 4H); ¹³C NMR (300 MHz, CD₃OD): δ 175.2, 174.6, 172.9, 149.9, 139.9, 139.1, 138.2, 137.6, 135.5, 129.5, 129.1, 128.0, 123.3, 123.0, 121.7, 117.8, 37.8, 33.7, 3037, 29.9, 29.8, 26.7, 26.6, 18.9. HRMS-FAB (M+1) calcd for C₂₆H₃₁N₄O₄ 463.2345. found 463.2351.

### N¹-hydroxy-N⁸-(4-(2-methyl-1,3-dioxo-1,3-bis(quinolin-8-ylamino)propan-2-yl)phenyl)octanediamide (76)

A suspension of ester 73 (100 mg, 0.158 mmol), hydroxylamine (50% solution in water, 0.6 mL) and catalytic amount of KCN (0.5 mg) in a co-solvent (MeOH:THF=2 mL:2 mL) was stirred at 35° C. to 40° C. for 24 h. After removing the solvent, the residue was treated with NH₄Cl (sat. aq.) to pH=4-5. The mixture was extracted with a co-solvent (CHCl₃: i-PrOH=4:1), dried over Na₂SO₄ and filtered. The filtrate was concentrated in vacuo and the residue was purified by column chromatography on a silica gel (CH₂Cl₂:MeOH=1:40-1/10) to give target hydroxamic acid 76 (37 mg, 37%). ¹H NMR (300 MHz, CD₃OD): δ 8.77-8.74 (m, 2H), 8.65-8.63 (m, 2H), 8.29-8.26 (m, 2H), 7.71-7.47 (m, 10H), 2.39 (t, 2H, J=7.5 Hz), 2.22 (s, 3H), 2.09 (t, 2H, J=7.5 Hz), 1.75-1.58 (m, 4H), 1.48-1.32 (m, 4H); ¹³C NMR (300 MHz, CD₃OD): δ 174.7, 172.9, 172.6, 149.9, 140.0, 137.5, 137.1, 135.3, 129.4, 129.2, 128.0, 123.7, 123.1, 121.8, 117.7, 63.1, 37.9, 33.7, 29.9, 29.8, 26.6, 24.1. HRMS-FAB (M+1) calcd for C₃₆H₃₇N₆O₅ 633.2825. found 633.2824.

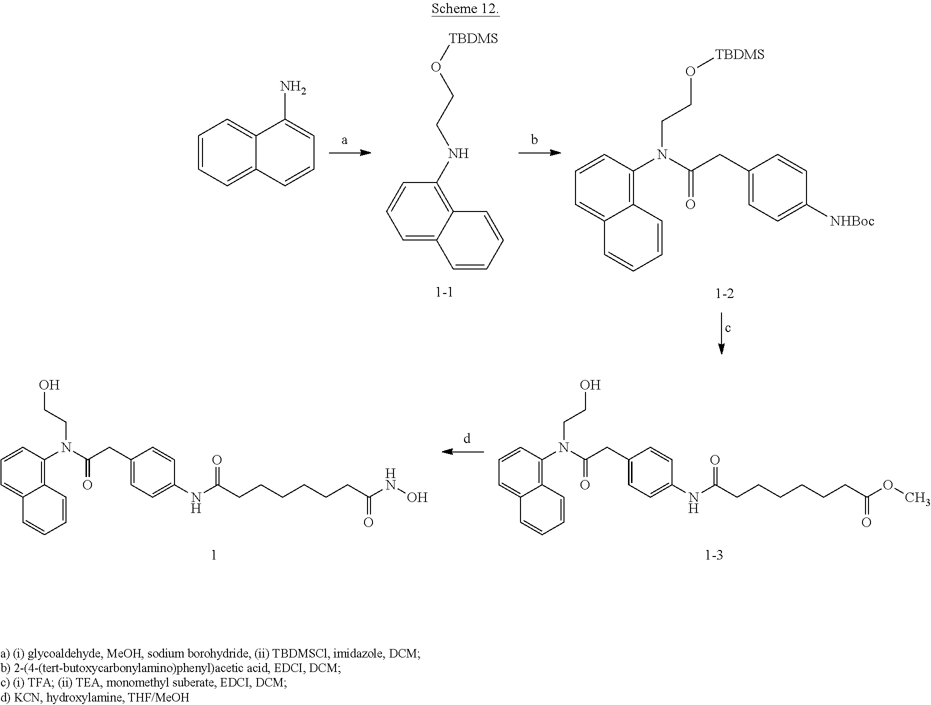

### N-(2-(tert-Butyldimethylsilyloxy)ethyl)naphthalen-1-amine (1-1)

1-naphthylamine (500 mg, 3.5 mmol) and glycoaldehyde (210 mg, 3.5 mmol) were mixed in MeOH (15 mL) at RT under argon. When no more 1-naphthylamine was detected by TLC, the aldimine was carefully treated with solid NaBH₄ (212 mg, 5.6 mmol). The reaction mixture was stirred for 10 minutes and quenched with 1 M NaOH. The product was then extracted with ether. The ether phase was then washed with sat. NaCl solution and dried with sodium sulfate. The product was then concentrated in vacuo and the residue purified by column chromatography on silica gel (Hexanes:EtOAC=10:1-5:1) to give 385 mg of product that was directly used in the next step. The 385 mg was dissolved in DCM (7 mL). The solution was then treated with TBDMS (339 mg, 2.26 mmol) and imidazole (209 mg, 3.08 mmol) at RT overnight under argon. The reaction was then washed with NH₄Cl and dried with sodium sulfate. The solution was then concentrated in vacuo to give 1-1 (519 mg, 2-step yield 60%). ¹H NMR (300 MHz, CDCl₃) δ 7.851-7.812 (br, 2H), 7.465 (s, 2H), 7.356 (s, 1H), 7.283-7.262 (br, 2H), 6.696 (s, 1H), 4.005 (s, 2H), 3.394 (s, 2H), 0.947 (s, 9H), 0.122 (s, 6H).

### tert-Butyl 4-(2-((2-(tert-butyldimethylsilyloxy)ethyl)(naphthalen-1-yl)amino)-2-oxoethyl)phenylcarbamate (1-2)

A mixture of 1-2 (519 mg, 1.72 mmol) and 2-(4-(tert-butoxycarbonylamino)phenyl)acetic acid (645 mg, 2.58 mmol) in DCM (10 mL) was treated with EDCI (492 mg, 2.58 mmol) at RT overnight. The reaction was washed with sat. NH₄Cl solution and the organic phase dried with sodium sulfate. The solution was concentrated in vacuo and purified by column chromatography on silica gel (Hexanes:EtOAc 20:1-10:1) to give 1-2 (696 mg, 1-step yield 76%). ¹H NMR (300 MHZ, CDCl₃) 7.912 (m, 2H), 7.786 (br, 1H), 7.571-7.536 (m, 2H), 7.454 (t, J=5.4, 1H), 7.153 (d, J=6 Hz, 2H), 6.845 (d, J=6 Hz, 2H), 6.358 (s, 1H), 4.372-4.311 (m, 1H), 3.879-3.761 (m, 2H), 3.477-3.413 (m, 1H), 3.229 (s, 1H), 1.593-1.510 (m, 14H), 0.845-0.818 (m, 9H), 0.012-0.009 (m, 6H).

### Methyl 8-(4-(2-((2-hydroxyethyl)(naphthalen-1-yl)amino)-2-oxoethyl)phenylamino)-8-oxooctanoate (1-3)

Compound 1-2 (696 mg, 1.301 mmol) was dissolved in TFA (6 mL) at RT for 30 min. The volatile was removed and the residue was coevaporated with toluene (10 mL×2) and EtOH (10 mL×2). The product was then directly used in the next step. The product (308 mg, 0.962 mmol) was dissolved in DCM (4 mL) and treated with TEA (402 μL, 2.82 mmol) followed by adding monomethyl suberate (277 μL, 1.07 mmol) and EDCI (204 mg, 1.07 mmol). The reaction was quenched with MeOH and the mixture dissolved in a mixed solvent (CHCl₃:i-PrOH 4:1, 20 mL) and washed with sat. NH₄Cl solution. The product was dried with sodium sulfate and concentrated in vacuo. The residue was the purified by column chromatography on silica gel (EtOAc:Hexanes 1:1-2:1-4:1) to give 1-3 (217 mg, 2 step yield 68.2%). ¹H NMR (400 MHz, CDCl₃) 7.940 (m, 2H), 7.844 (s, 1H), 7.596 (d, J=1.5 Hz, 2H), 7.488 (m, 1H), 7.336 (s, 2H), 7.096 (s, 1H), 6.891 (m, 2H), 5.312 (m, 1H), 4.365 (br, 1H), 3.837-3.826 (m, 2H), 3.678 (m, 3H), 3.666 (m, 1H), 3.315 (br, 2H), 2.338 (br, 4H), 1.385 (s, 4H), 1.274 (m, 1H). HR-MS for (C₂₉H₃₄N₂O₅+H) 491.5907. found 491.2544.

### N¹-hydroxy-N⁸-(4-(2-((2-hydroxyethyl)(naphthalen-1-yl)amino)-2-oxoethyl)phenyl)octanediamide (1)

Compound 1-3 (217 mg, 0.443 mmol) was dissolved in THF/MeOH (1 mL/1 mL) and treated with hydroxylamine (1.5 mL, 50% water solution) in the presence of cat. KCN (2 mg, 5%). When no more 1-3 could be detected, the solution was acidified to pH 4 and the mixture was dissolved in a mixed solvent (CHCl₃:i-PrOH 4:1, 20 mL) and washed with sat. NH₄Cl solution. The organic phase was dried with sodium sulfate and concentrated in vacuo. The residue was then purified by column chromatography on silica gel (DCM:MeOH 20:1-10:1) to give target hydroxamic acid 1 (184 mg, 1 step yield 88.4%) ¹H NMR (400 MHz, DMSO-d6) 10.314 (s, 1H), 9.740 (s, 1H), 8.637 (d, J=1.2 Hz, 1H), 8.075-8.016 (m, 2H), 7.802-7.778 (m, 1H), 7.642-7.513 (m, 5H), 7.386 (d, 2H), 6.808 (d, J=0.9 Hz, 2H), 4.660 (t, 0.9 Hz, 1H), 4.211-4.146 (m, 1H), 4.092 (m, 1H), 3.514 (m, 2), 3.188-3.150 (m, 2H), 3.069 (s, 2H), 2.252 (t, J=5.7 Hz and 5.4 Hz, 2H), 1.935 (t, J=5.4 Hz and 5.7 Hz, 2H), 1.569-1.467 (m, 5H), 1.272 (br, 5H). HR-MS for (C₂₈H₃₃N₃O₅+H) 492.5787. found 492.2502.

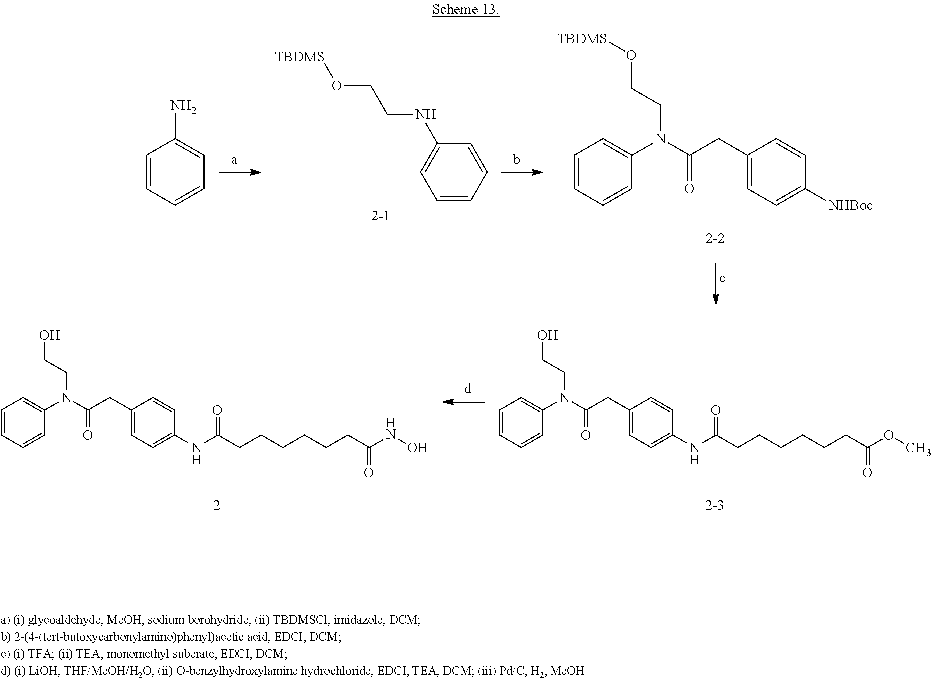

### N-(2-(tert-Butyldimethylsilyloxy)ethyl)aniline (2-1)

Aniline (0.489 mL, 5.37 mmol) and glycoaldehyde (322 mg, 5.37 mmol) were mixed in MeOH (20 mL) at RT under argon for five hours. The aldimine was then carefully treated with solid NaBH₄ (304.5 mg, 8.06 mmol). The reaction was stirred for 10 minutes and quenched with 1 M NaOH. The product was extracted with ether then washed with sat. NaCl solution. The organic phase was then dried with sodium sulfate and purified by column chromatography on silica gel (Hexanes:EtOAc 10:1-5:1-1:1) to give 378 mg of product that was directly used for the next step. 110 mg of aniline were recovered. The 378 mg of product was dissolved in DCM (7 mL). The solution was treated with TBDMS (451 mg, 3.01 mmol) and imidazole (279.5 mg, 4.1 mmol). When no starting material remained, the reaction mixture was washed with NH₄Cl solution and the organic phase dried with sodium sulfate. The solution was concentration in vacuo to give 2-1 (612 mg, 2-step yield 58.3%). ¹H NMR (300 MHz, CDCl₃), 7.306-7.200 (m, 2H), 6.733-6.671 (m, 3H), 3.853 (t, J=5.21 and 3.6 Hz, 2H), 3.254 (t, J=5.1 and 3.6 Hz, 2H), 0.944 (s, 9H), 0.103 (s, 6H).

### tert-Butyl 4-(2-((2-(tert-butyldlmethylsilyloxy)ethyl)(phenyl)amino)-2-oxoethyl)phenylcarbamate (2-2)

Compound 2-1 (612 mg, 2.44 mmol) and 2-(4-(tert-butoxycarbonylamino)phenyl)acetic acid (915 mg, 3.66 mmol) were dissolved in DCM (12 mL) and treated with EDCI at RT overnight. The reaction mixture was washed with NH₄Cl solution and the organic phase dried with sodium sulfate. The solution was concentrated in vacuo and purified by column chromatography on silica gel (Hexanes:EtOAc 40:1-20:1-10:1) to give 2-2 (697 mg, 1-step yield 58.9%). ¹H NMR (300 MHz, CDCl₃), 7.386-7.367 (br, 3H), 7.241 (d, J=6.9 Hz, 2H), 7.179 (br, 2H), 6.993 (br, 2H), 3.794 (s, 4H), 3.390 (s, 2H), 0.859 (s, 9H), 0.026 (s, 6H).

### Methyl 8-(4-(2-((2-hydroxyethyl)(phenyl)amino)-2-oxoethyl)phenylamino)-8-oxooctanoate (2-3)

Compound 2-2 (0.697 mg, 1.44 mmol) was dissolved in TFA (7 mL). The reaction was run for 30 minutes. The volatile was removed and the residue co-evaporated with toluene (11 mL×2) and EtOH (11 mL×2). The residue was then directly used in the next step. The residue (285 mg, 1.052 mmol) was dissolved in DCM (4 mL) and treated with TEA (439 μL, 3.156 mmol) followed by adding monomethyl suberate (209 μL, 1.157 mmol) and EDCI (221 mg, 1.157 mmol). The reaction was quenched with MeOH and the mixture dissolved in a mixed solvent (CHCl₃:i-PrOH 4:1, 20 mL) and washed with sat. NH₄Cl solution. The product was dried with sodium sulfate and concentrated in vacuo. The residue was the purified by column chromatography on silica gel (Hexanes:EtOAc 2:1-1:1-1:2) to give 2-3 (146 mg, 2-step yield 22.9%). ¹H NMR 7.440 (t, J=6.9 and 7.8, 5H), 7.280 (d, J=7.5, 2H), 6.973 (d, J=8.1), 3.866-3.825 (m, 2H), 3.662 (s, 6H), 3.436 (s, 2H), 2.385-2.317 (m, 4H), 1.730-1.623 (m, 5H), 1.412 (s, 4H), 0.021 (s, 5H).

### N¹-Hydroxy-N⁸-(4-(2-((2-hydroxyethyl)(phenyl)amino)-2-oxoethyl)phenyl)octanediamide (2)

Compound 2-3 (146 mg, 0.33 mmol) was dissolved in THF:MeOH:H₂O (2:1:1, 2.8 mL). The solution was treated with LiOH.H₂O (27.7 mg, 0.66 mmol). The reaction was neutralized to pH=4-5 after 5 hours. The solution was extracted with a mixed solvent (CHCl₃:i-PrOH 4:1, 12 mL×4) and evaporated in vacuo. The residue was then directly used in the next step. The residue (80 mg, 0.188 mmol) was dissolved in DCM (4 mL) and treated with TEA (105 μL, 0.752 mmol) and O-benzylhydroxamic hydrochloride (32.8 mg, 0.207 mmol) and EDCI (43.7 mg, 0.228 mmol). The reaction was run until no more 2-3 was detected by TLC. The solution was the washed with NH₄Cl solution and the organic phase dried with sodium sulfate. The product was purified by column chromatography on silica gel (DCM:MeOH 40:1-20:1-10:1). The residue was then directly used in the next step. The residue (13 mg, 0.025 mmol) was dissolved in MeOH (2 mL) and treated with Pd/C (5 mg). An H₂ balloon was then added. The reaction was run until no more of the original residue was detected by TLC. The reaction was then quenched with celite and filtered. The solution was then concentrated in vacuo and purified by preparative TLC (DCM:MeOH 9:1) to give 2 (5.2 mg, 3 step yield 3.57%). ¹H NMR (300 MHz, MeOD), 7.465-7.414 (m, 5H), 7.277 (d, J=6.6, 2H), 6.972 (d, J=8.1, 2H), 3.865-3.826 (m, 2H), 3.688-3.668 (m, 2H), 3.435 (s, 2H), 2.388-2.338 (m, 2H), 2.107-2.084 (m, 2H), 1.668-1.644 (m, 5H), 0.917 (m, 2H). HR-MS for (C₂₄H₃₁N₃O₅+H) 442.5204. found 442.2356.

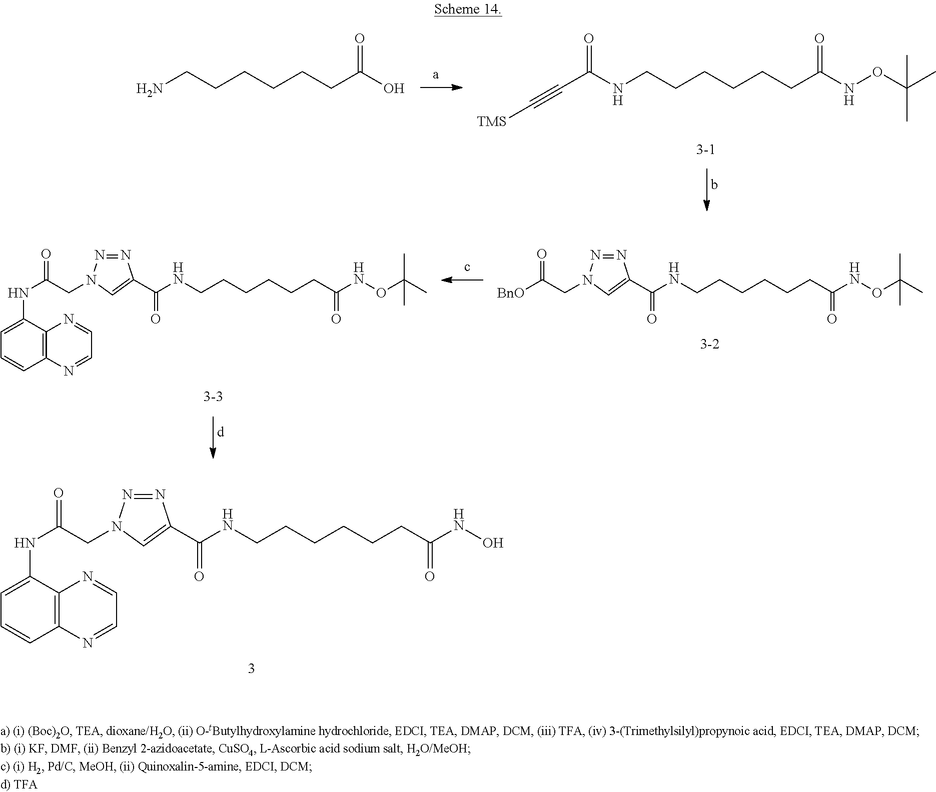 [SMILES: NCCCCCCC(=O)O ; CC(C)(C)ONC(=O)CCCCCCNC(=O)C#C[Si](C)(C)C ; CC(C)(C)ONC(=O)CCCCCCNC(=O)c1cn(CC(=O)Nc2cccc3nccnc23)nn1 ; CC(C)(C)ONC(=O)CCCCCCNC(=O)c1cn(CC(=O)OCc2ccccc2)nn1 ; O=C(CCCCCCNC(=O)c1cn(CC(=O)Nc2cccc3nccnc23)nn1)NO]

### N-tert-Butoxy-7-propiolamidoheptanamide (3-1)

7-Aminohepatonic acid (726 mg, 5 mmol) was dissolved in a mixture solvent of H₂O (3 mL)/dioxane (3 mL). To this solution, Et₃N (2.08 mL, 15 mmol) was added followed by di-tert-butyl dicarbonate (1.72 mL, 7.5 mmol). The reaction mixture was kept room temperature and stirred for 7 hr. The After removed the organic solvent in vacuo, the solution was acidified by adding HCl solution (3N) to pH˜1.0 and extracted with EtOAc. The organic layer was collected and evaporated to give syrup which was treated with NaOH (1N) to pH˜11.0 and washed with EtOAc. The aqueous phase was acidified to pH˜1.0 and extracted with EtOAc. The organic layer was dried and evaporated to give syrup and was directly used for the next step. The above syrup was dissolved in CH₂Cl₂ (50 mL) and treated with O-tButylhydroxylamine hydrochloride (1.00 g, 8.0 mmol), EDCI (1.59 g, 8.3 mmol), TEA (1.14 mL, 8.2 mmol), DMAP (183 mg, 1.5 mmol). After stirred at room temperature for 1.5 hr, the reaction mixture was washed with HCl (1N) and the organic layer was collected and dried and evaporated to give syrup (1.57 g). The pale yellow syrup (1.30 g) was dissolved in TFA (10 mL) and stirred for 30 min. The excess TFA was completely removed in vacuo and the syrup was dissolved in CH₂Cl₂ (50 mL) and treated with 3-(Trimethylsilyl)propynoic acid (701 mg, 5.0 mmol), EDCI (945 mg, 4.9 mmol), TEA (2.27 mL, 16.4 mmol), DMAP (100 mg, 0.82 mmol). After stirred at 50° C. for 18 hr, the reaction mixture was washed with HCl (IN) and dried over Na₂SO₄ and filtered. The filtrate was concentrated in vacuo and the residue was purified by column chromatography on a silica gel (EtOAc:Hexanes=1:1-1:2) to give a foam as compound 3-1 (600 mg, 4-step yield 42%). ¹H NMR (300 MHz, CDCl₃) δ 3.25 (dt, J=6.9 and 17.6 Hz, 2H), 2.12 (br, 2H), 1.64 (br, 2H), 1.51 (br, 2H), 1.33 (br, 4H), 1.25 (s, 9H), 0.21 (s, 9H).

### Benzyl 2-(4-(7-(tert-butoxyamino)-7-oxoheptylcarbamoyl)-1H-1,2,3-triazol-1-yl)acetate (3-2)

Compound 3-1 (68 mg, 0.2 mmol) was dissolved in DMF (1 mL) and treated with KF (15.1 mg, 0.26 mmol) at −10° C. The temperature was allowed to warm up to 0° C. during 25 min before it was quenched with NH₄Cl solution. The mixture was extracted with CH₂Cl₂ and washed with NH₄Cl. The organic layer was dried and evaporated to dry. The residue was and benzyl 2-azidoacetate (76 mg, 0.6 mmol) was dissolved in a mixture solvent of EtOH (0.8 mL)/H₂O (0.8 mL). The L-Ascorbic acid sodium salt (8 mg, 0.04 mmol) and CuSO₄ (5 mg, 0.031 mmol) were added into the solution and stirred for 18 hr. After extracted with EtOAc, the organic phase was dried and purified by column chromatography on silica gel (EtOAc:Hexanes=4:1—pure EtOAc to EtOAc:MeOH=100:1) to give triazole 3-2 (40 mg, 44%). ¹H NMR (400 MHz, DMSO-d6) δ 10.23 (s, 1H), 8.58 (d, J=5.6 Hz, 1H), 8.54 (s, 1H), 7.42-7.34 (m, 5H), 5.54 (s, 2H), 5.22 (s, 2H), 3.23 (dt, J=6.4 and 6.8 Hz, 2H), 2.00 (t, J=7.2 Hz, 2H), 1.50 (br, 2H), 1.28 (br, 2H), 1.13 (s, 9H); HR-MS Calcd. for (C₂₃H₃₃N₅O₅+H) 460.2560. found 460.2565.

### N-(7-(tert-butoxyamino)-7-oxoheptyl)-1-(2-oxo-2-(quinoxalin-5-ylamino)ethyl)-1H-1,2,3-triazole-4-carboxamide (3-3)

Compound 3-2 (350 mg, 0.76 mmol) was dissolved in MeOH (5 mL) and treated with Pd/C in the presence of an H₂ balloon. The reaction was kept at room temperature for 45 min and filtered. The filtrate was concentrated in vacuo to give a residue. The residue and quinoxalin-5-amine (205 mg, 0.71 mmol) were dissolved in CH₂Cl₂ (10 mL) and treated with EDCI (150 mg, 0.78 mmol) at room temperature for overnight. The reaction mixture was washed with NH₄Cl solution and dried over Na₂SO₄ and filtered. The filtrate was concentrated in vacuo and the residue was purified by column chromatography on a silica gel (MeOH:CH₂Cl₂=1:30-1:15) to give a foam 3-3 (170 mg, 2-step yield 45%). ¹H NMR (400 MHz, DMSO-d6) δ 10.88 (s, 1H), 10.17 (s, 1H), 9.06 (d, J=2.4 Hz, 1H), 9.00 (d, J=2.4 Hz, 1H), 8.60-8.57 (m, 1H), 8.54 (s, 1H), 8.50 (t, J=7.6 Hz, 1H), 7.85-7.83 (m, 2H), 5.69 (s, 2H), 3.22 (dt, J=8.8 and 17.6 Hz, 2H), 1.97 (t, J=9.6 Hz, 2H), 1.50-1.46 (m, 4H), 1.28-1.27 (m, 4H), 1.12 (s, 9H). HR-MS Calcd. for (C₂₄H₃₂N₈O₄+H) 497.2625. found 497.2596.

### N-(7-(hydroxyamino)-7-oxoheptyl)-1-(2-oxo-2-(quinoxalin-5-ylamino)ethyl)-1H-1,2,3-triazole-4-carboxamide (3)

Compound 3-3 (70 mg, 0.14 mmol) was dissolved in TFA (3 mL) and stirred at room temperature for 48 h. After completely removed the volatile, the residue was dissolved in a co-solvent (CHCl₃:i-PrOH=4:1) and washed with buffer (pH 7.0), dried over Na₂SO₄ and filtered. The filtrate was concentrated in vacuo and the residue was purified by column chromatography on silica gel (CH₂Cl₂:MeOH=1:20-1/10) to give target hydroxamic acid 3 (15 mg, 40% after recovered 10 mg of starting material). ¹H NMR (400 MHz, DMSO-d₆) δ 10.88 (s, 1H), 10.29 (s, 1H), 9.06 (d, J=2.4 Hz, 1H), 8.60-8.48 (m, 4H), 7.85-7.83 (m, 2H), 5.69 (s, 2H), 3.22 (dt, J=9.2 and 18.4 Hz, 2H), 1.92 (t, J=9.6 Hz, 2H), 1.50-1.47 (m, 4H), 1.28-1.27 (m, 4H), 1.12 (s, 9H). HR-MS Calcd. for (C₂₀H₂₄N₈O₄+H) 441.1999. found 441.2011.

### Example 2

### IC₅₀ Values for the Inhibition of HDAC6 and HDAC1

Various compounds were tested in in vitro enzyme assays for their activity in inhibiting HDAC6 and HDAC1. The results are set forth below. The assays used was a fluorogenic HDAC assay kit (BPS Bioscience, San Diego, Calif.). On a micotiter the HDAC fluorometric substrate containing an aceylated side chain is incubated with a sample containing HDAC activity (purified/recombinant HDAC1 or HDAC6 enzyme). The deacetylation sensitizes the substrate so subsequent treatment with a lysine developer produces a fluorophore that can then be measured using a fluorescence reader. The assay is performed in the absence and presence of the potential inhibitor compound.

| | | | | Cmpnd. # |
|---|---|---|---|---|
| | IC₅₀ on | IC₅₀ on | IC₅₀ | and |
| | HDAC6 | HDAC1 | 0 ratio | Synthetic |
| Compound | (nM) | (nM) | (1/6) | Scheme |
| 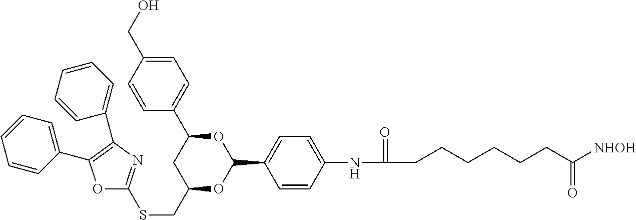 | 90 | 612 | 6.8 | Tubacin |
| 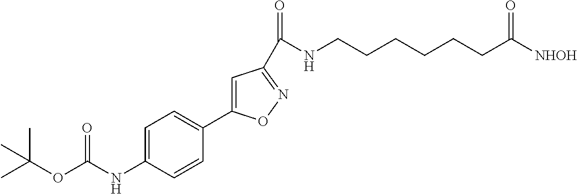 | 5.8 | 38.4 | 6.6 | Kozikowski compound |
| 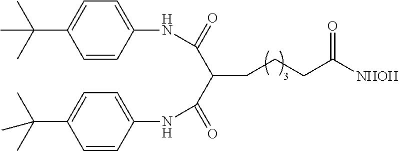 | 34.4 | 106.2 | 3.1 | (77) |
| 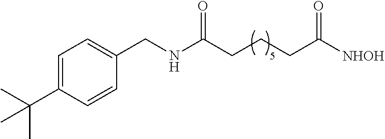 | 65.7 | 676.3 | 10.3 | (78) |
| 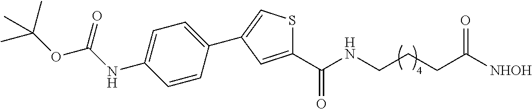 | 4.4 | 46.7 | 10.6 | (79) |
| 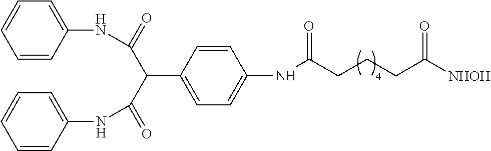 | 3 | 128.6 | 42.9 | (9) Scheme 2 |
| 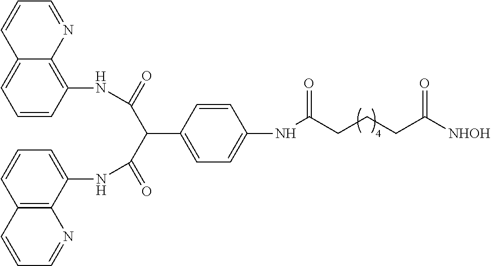 | 2.25 | 242.3 | 107 | (15) Scheme 2 and (6) Scheme 1 |
| 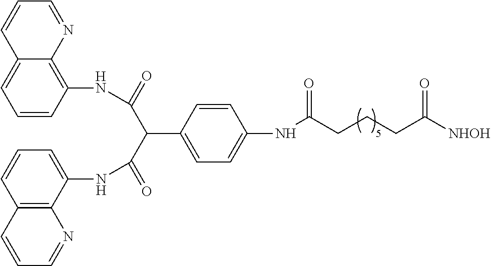 | 57 | 1445 | 25 | (28) Scheme 3 |
| 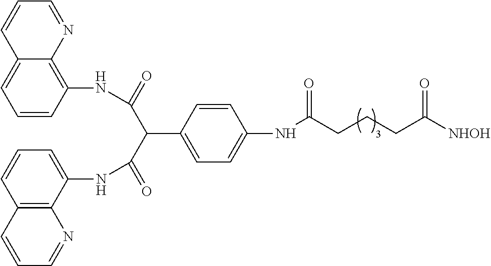 | 22 | 1130 | 51 | (31) Scheme 3 |
| 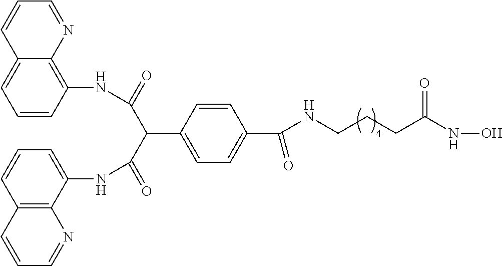 | 12 | 1043 | 87 | (61) Scheme 8 |
| 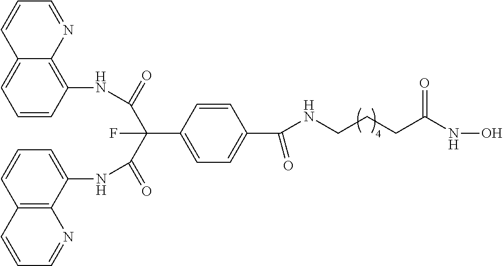 | 42 | 2446 | 58 | (65) Scheme 9 |

### Example 3

### LNCaP Cells were Cultured with the Following Compound

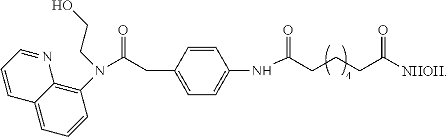

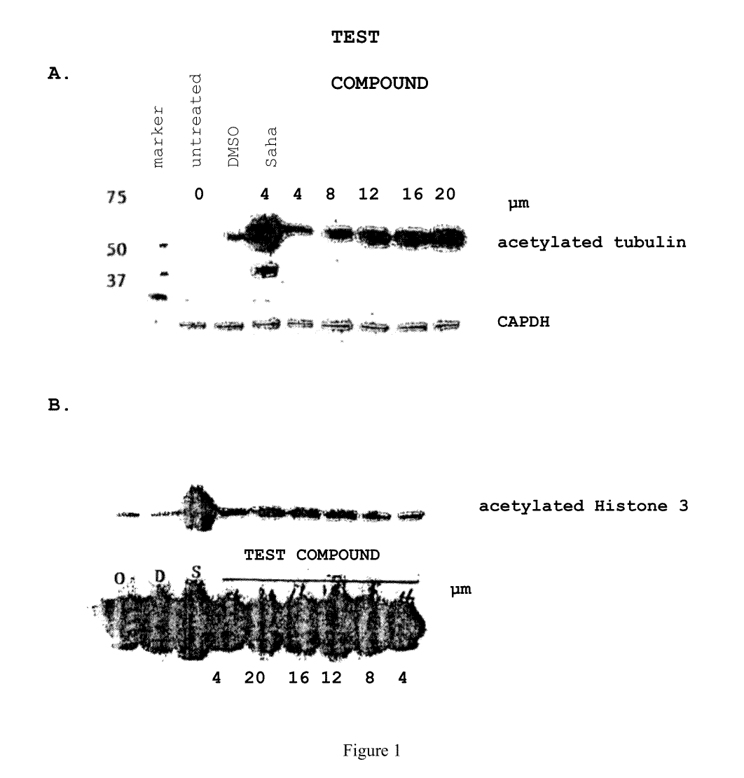

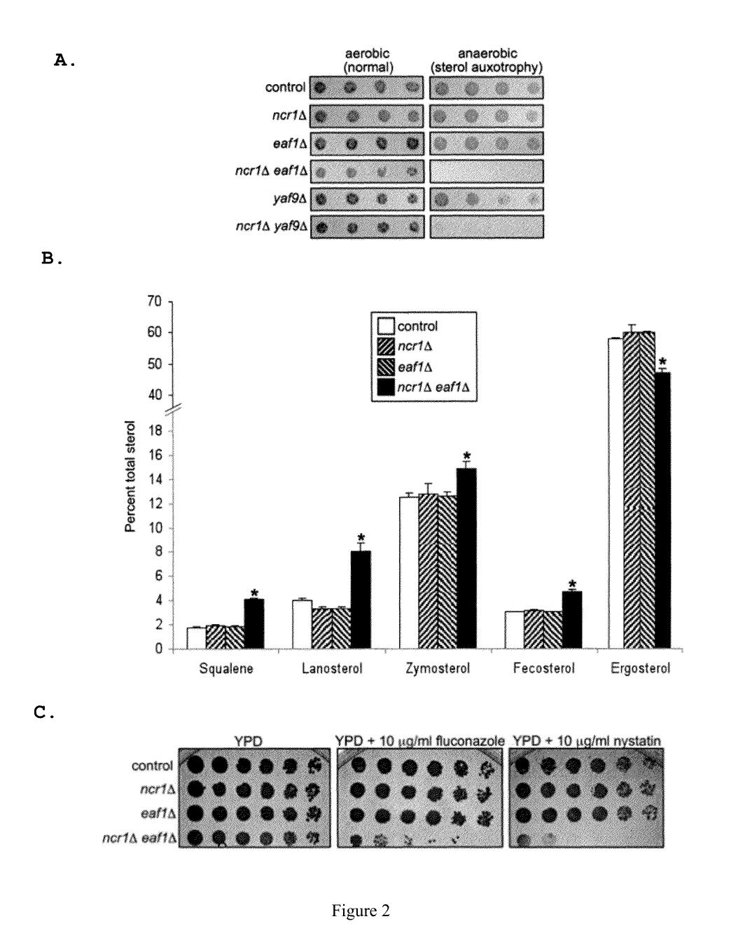

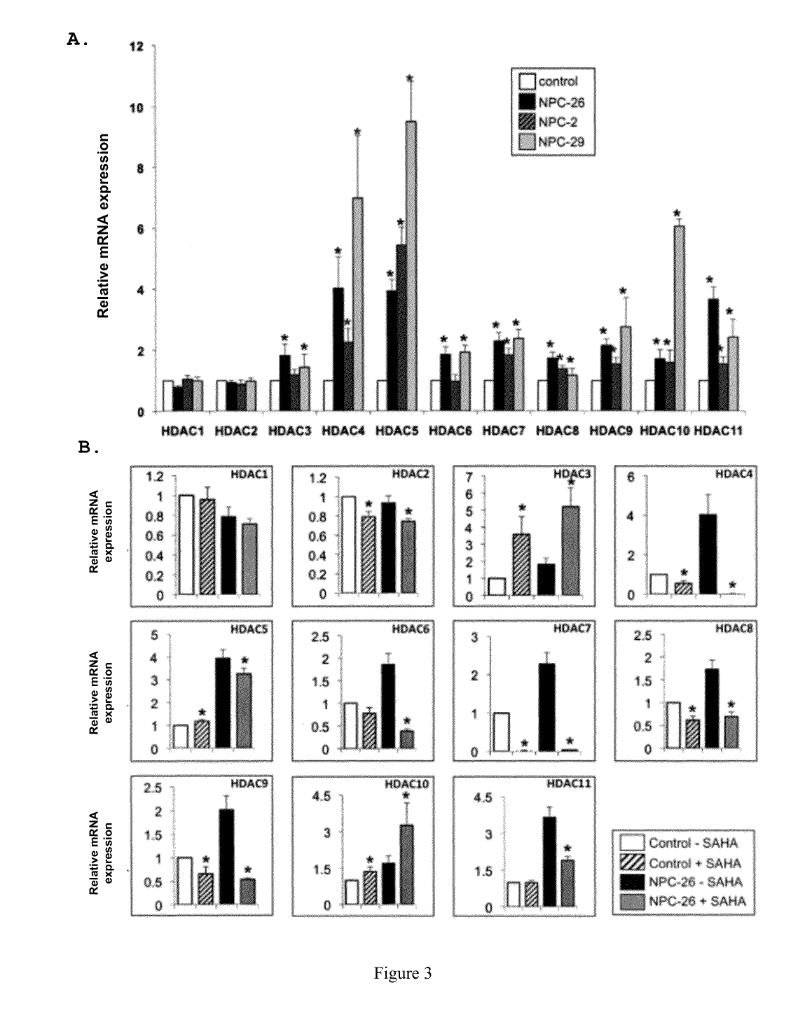

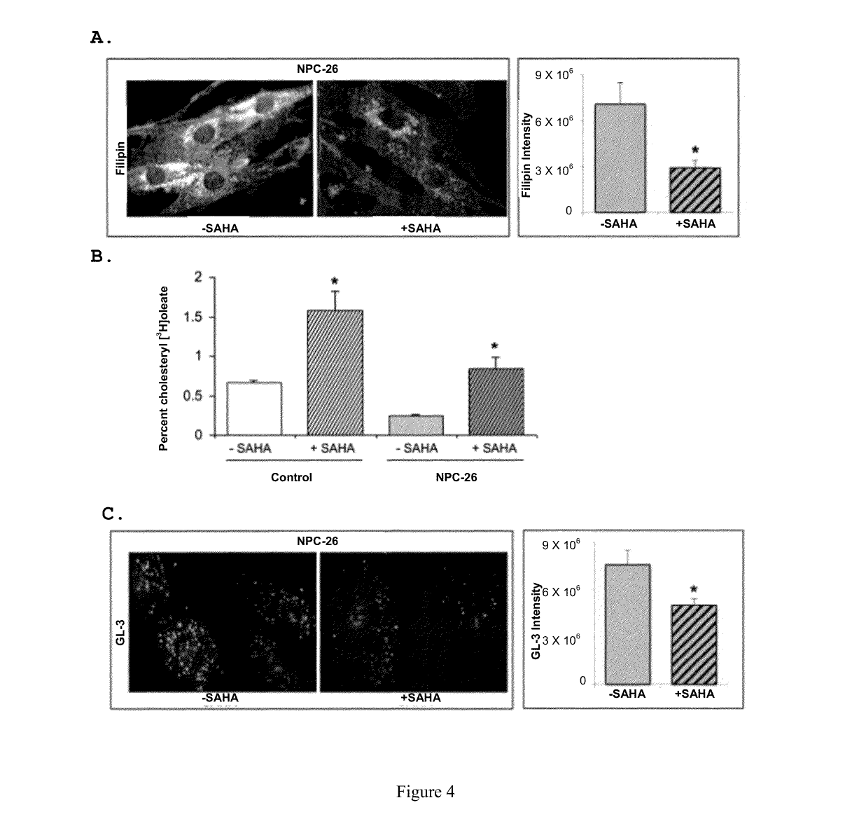

Staining of the cultured LNCaP cells showed induced accumulation of acetylated alpha-tubulin (See FIG. 1A), but not acetylated histone, H3 (See FIG. 1B). These results are consistent with this compound selectively inhibiting HDAC6 in this cell-based assay.

### Materials and Methods

### Cell Based Assay—Cell Growth & Viability

Adherent Cells: LNCaP (human prostate cancer cells), HFS (human foreskin fibroblasts)
Suspension Cells: MELC (murine erythroleukemia cells)

### Adherent Cells:

1. Seed 5×104 cells/well (24 well tissue culture plate) and allow cells to adhere overnight (18 h)
2. Treat cells with drug(s) as indicated, Treatment time=Time 0
3. Harvest cells using trypsin at 0, 24, 48, 72 h after treatment and count manually with hemocytometer using trypan blue dye exclusion.

### Suspension Cells:

1. For using log phase growing cells, suspension cells are split 4×105 cells/ml day before experiment.
2. Treat cells (1×105 cells/ml) with drug(s) as indicated, Treatment time=Time 0
3. At times indicated, aliquots of cells are taken for cell growth and viability counts.
a. 100 μL used for Coulter Counter cell growth counts
b. 40 μL used with hemocytometer & trypan blue dye exclusion viability counts
4. Lyse cells with RIPA buffer or Histone Lysis Buffer (50 μL/2×106 cells) and separate soluble and insoluble fractions.
a. Determine soluble protein concentration using Bradford Assay
b. Extract histones from insoluble fraction & determine histone protein concentration using Bradford Assay
5. Run Nu-PAGE Bis-Tris 4-12% gel (Invitrogen, Carlsbad, Calif.; run as per company instructions) electrophoresis and perform western blotting analysis with indicated antibodies.
a. MOPS buffer used for soluble fraction proteins
b. MES buffer used for histone protein fraction

### Cell Based Assay—Immunoblots

### Adherent Cells:

1. Seed 1×106 cells/dish (10 cm tissue culture dish) and allow cells to adhere overnight (18 h)
2. Treat cells with drug(s) as indicated, Treatment time=Time 0
3. Harvest cells using trypsin at times indicated after treatment
4. Lyse cells with RIPA buffer (modification of Taunton et al.) or Histone Lysis Buffer (Aton Biotech) 50 μL/2×106 cells) and separate soluble and insoluble fractions.
a. Determine soluble protein concentration using Bradford Assay (BioRad, Hercules, Calif.)
b. Extract histones from insoluble fraction as per protocol established by Aton Biotech & determine histone protein concentration using Bradford Assay (BioRad, Hercules, Calif.)
5. Run Nu-PAGE Bis-Tris 4-12% gel (Invitrogen, Carlsbad, Calif.; run as per company instructions) electrophoresis and perform western blotting analysis with indicated antibodies.
a. MOPS buffer used for soluble fraction proteins
b. MES buffer used for histone protein fraction

### In Vitro Enzymatic Assay

Follow BPS Bioscience Fluorogenic HDAC Assay Kit (see Example 2). Range of inhibitor compounds tested: 10,000-0.1 nM

### REFERENCES

1. R. B. Parmigiani, W. S. Xu, G. Venta-Perez, H. Erdjument-Bromage, M. Yaneva, P. Tempst, and P. A. Marks. “HDAC6 is a specific deacetylase of peroxiredoxins and is involved in redox regulation” Proc. Nat. Acad. Sci. USA (2008), 105, 9633-9638.
2. K. V. Butler and A. P. Kozikowski “Chemical Origins of Isoform Selectivity in Histone Deacetylase Inhibitors” Curr. Pharma. Design (2008), 14, 505-528.
3. Y. Kawaguchi; J. J. Kovacs; A. McLaurin; J. M. Vance; A. Ito; T.-P. Yao “The Deacetylase HDAC6 Regulates Aggresome Formation and Cell Viability in Response to Misfolded Protein Stress” Cell (2003), 115, 727-738.
4. P. Bali; M. Pranpat; J. Bradner; M. Balasis; W. Fiskus; F. Guo; K. Rocha; S. Kumaraswamy; S. Boyapalle; P. Atadja; E. Seto; K. Bhalla. “Inhibition of Histone Deacetylase 6 Acetylates and Disrupts the Chaperone Function of Heat Shock Protein 90” J. Biol. Chem. (2005), 280, 26729-26734.
5. Y.-S. Gao; C. C. Hubbert; T.-P. Yao. “The Microtubule-associated Histone Deacetylase 6 (HDAC6) Regulates Epidermal Growth Factor Receptor (EGFR) Endocytic Trafficking and Degradation” J. Biol. Chem. (2010), 285, 11219-11226.
6. S. J. Haggarty; K. M. Koeller; J. C. Wong; C. M. Grozinger; S. L. Schreiber. “Domain-selective small-molecule inhibitor of histone deacetylase 6 (HDAC6)-mediated tubulin deacetylation” Proc. Nat. Sci. Acad. USA (2003), 100, 4389-4394.
7. J. J. Kovacs; P. J. M. Murphy; S. Gaillard; X. Zhao; T. Wu; C. V. Nicchitta; M. Yoshida; D. O. Toft; W. B. Pratt; T.-P. Yao. “HDAC6 Regulates Hsp90 Acetylation and Chaperone-Dependent Activation of Glucocorticoid Receptor” Molecular Cell (2005), 18, 601-607.
8. Marks, P. S., Breslow, R. Dimethyl sulfoxide to vorinostat: development of this histone deacetylase inhibitor as an anti-cancer drug. Nat. Biotech, (2007) 25:84-90.
9. Marks, P. A. histone Deacetylase Inhibitors: A chemical approach to understanding cellular functions, Biochimica et Biophysicia Acta (in press, 2010).
10. Munkacsi, Andrew B. et al., “An “exacerbate-reverse” strategy in yeast identifies histone deacetylase inhibition as a correction for cholesterol and sphingolipid transport defects in human niemann-pick type C disease”, The Journal of Biological Chemistry, published on Apr. 13, 2011 at http://www.jbc.org/cgi/doi/10.1074/jbc.M111.227645.

1. A compound having the structure 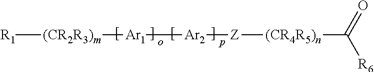
wherein
R₁ is H, halogen, —NR₇R₈, —NR₅₁—C(═O)—R₅₂, —NH—C(═O)—OR₇, —OR₇, —NO₂, —CN, —SR₇, —SO₂R₇, —CO₂R₇, CF₃, —SOR₇, —POR₇, —C(═S)R₇, —C(═O)—NR₇R₈, —CH₂—C(═O)—NR₇R₈, —C(═NR₇)R₈, —P(═O)(OR₇)(OR₈), —P(OR₇)(OR₈), —C(═S)R₇, C₁₋₅ alkyl, C₂₋₅ alkenyl, C₂₋₅alkynyl, aryl, heteroaryl, or heterocyclyl,
wherein R₇, R₈, R₅₁ and R₅₂ are each, independently, H, C₁₋₅ alkyl, C₂₋₅ alkenyl, C₂₋₅ alkynyl, aryl, or heteroaryl;
m is an integer from 0 to 5;
R₂ and R₃ are each, independently, H, —(NH₂), —CH₂—R₉, —C(═O)OR₉, —C(═O)NR₉R₁₀, or —C(═O)R_{g},
wherein
R₉ and R₁₀ are each, independently, H, C₁₋₁₀ alkyl, C₂₋₁₀ alkenyl, C₂₋₁₀ alkynyl, —(CH₂)ᵣ—OR₁₈, cycloalkyl, 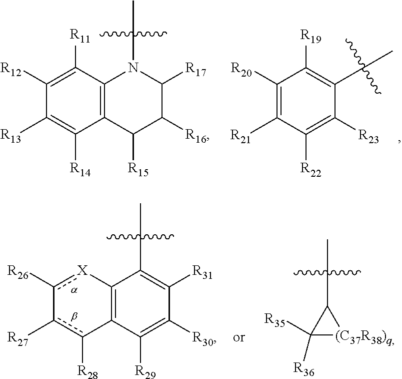
wherein
q is an integer from 1 to 6;
r is an integer from 1 to 10;
R₁₈ is H, C₁₋₁₀ alkyl, C₂₋₁₀ alkenyl, C₂₋₁₀ alkynyl, cycloalkyl, aryl, heteroaryl, or heterocyclyl;
bond α and bond β are each, independently, present or absent;
when bond α is present, X is N or CR₃₂;
when bond α is absent, X is NR₃₂ or CR₃₁R₃₂;
R₁₁, R₁₂, R₁₃, R₁₄, R₁₅, R₁₆, R₁₇, R₁₉, R₂₀, R₂₁, R₂₂, R₂₃, R₂₆, R₂₇, R₂₈, R₂₉, R₃₀, R₃₁, R₃₂, R₃₅, R₃₆, R₃₇, and R₃₈ are each, independently, H, halogen, —NO₂, —CN, —NR₂₄R₂₅, —SR₂₄, —SO₂R₂₄, —CO₂R₂₄, —OR₂₄, CF₃, —SOR₂₄, —POR₂₄, —C(═S)R₂₄, —C(═NR₂₄)R₂₅, —P(═O)(OR₂₄)(OR₂₅), —P(OR₂₄)(OR₂₅), —C(═S)R₂₄, C₁₋₁₀ alkyl, C₂₋₁₀ alkenyl, C₂₋₁₀ alkynyl, aryl, heteroaryl, or heterocyclyl;
wherein R₂₄ and R₂₅ are each, independently, H, C₁₋₁₀ alkyl, C₂₋₁₀ alkenyl, C₂₋₁₀ alkynyl, aryl, heteroaryl, or heterocyclyl;
wherein R₂ or R₃ is other than H;
Ar₁ and Ar₂ are each, independently, arylene or a heteroarylene other than isooxazolylene;
o and p are each, independently, 0 or 1;
Z is a bond, —NR₄₃—C(═O)—, or —C(═O)—NR₅₀—
wherein R₄₃ and R₅₀ are, independently, H, —(CR₄₄R₄₅)ₛ—CR₄₄R₄₅R₄₆, C₁₋₁₀ alkyl substituted by heterocyclyl, C₂₋₁₀ alkenyl, C₂₋₁₀ alkynyl, aryl, heteroaryl, or heterocyclyl,
wherein
s is an integer from 1 to 10;
R₄₄, R₄₅, and R₄₆ are each, independently, H, halogen, —NO₂, —CN, —NR₄₇R₄₈, —SR₄₇, —SO₂R₄₇, —CO₂R₄₇, —OR₄₇, CF₃, —SOR₄₇, —POR₄₇, —C(═S)R₄₇, —C(═NR₄₇)R₄₅, —P(═O) (OR₄₇)(OR₄₅), —P(OR₄₇)(OR₄₈), —C(═S)R₄₇, C₁₋₁₀ alkyl, C₂₋₁₀ alkenyl, C₂₋₁₀ alkynyl, aryl, heteroaryl, or heterocyclyl,
wherein R₄₇ and R₄₈ are each, independently, H, C₁₋₁₀ alkyl, C₂₋₁₀ alkenyl, C₂₋₁₀ alkynyl, aryl, heteroaryl, or heterocyclyl;
wherein when Z is a bond, o is 0, p is 0, and R₂ or R₃ is C(═O)NR₉R₁₀, then
R₉ and R₁₀ are each, independently, H, C₁₋₁₀ alkyl, C₂₋₁₀ alkenyl, C₂₋₁₀ alkynyl, —(CH₂)ᵣ—OR₁₈, 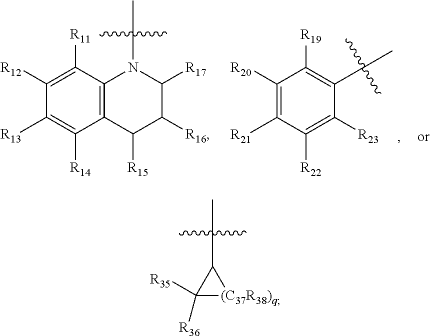
R₄ and R₅ are each, independently, H, C₁₋₁₀ alkyl, C₂₋₁₀ alkenyl, C₂₋₁₀ alkynyl, aryl, heteroaryl, or heterocyclyl;
n is an integer and is 0 or from 2 to 10;
R₆ is —OR₄₉ or —NH—OR₄₉,
wherein R₄₉ is H, C₁₋₁₀ alkyl, C₂₋₁₀ alkenyl, C₂₋₁₀ alkynyl, aryl, heteroaryl, or heterocyclyl;
wherein if R₁ is —NR₅₁—C(═O)—R₅₂ and m=0 and Ar₁ or Ar₂ is present and is bonded directly to R₁, then Ar₁ or Ar₂, respectively, is other than triazolyl;
wherein if Z is —NR₄₃—C(═O)—, and one of o and p is 0 and the other is 1, and m=1 or 0, then R₁ is other than —NR₇R₈, —NR₅₁—C(═O)—R₅₂, or —NH—C(═O)—OR₇;
wherein if Z is —C(═O)—NR₅₀— and n=5, and R₄ and R₅ are H and R₆ is NHOH and P and O are 1 and m=0 then R₁ is other than —NH—C(═O)—OR₇;
wherein when m=0 and Ar₁ or Ar₂ is bonded directly to R₁, then R₁ is other than H;
wherein when n=0, then p=0 and o=1, and R₁ is —C(═O)NR₇R₈;
wherein each occurrence of alkyl, alkenyl, or alkynyl is unsubstituted or substituted, branched or unbranched;
wherein each occurrence of cycloalkyl, aryl, heteroaryl, heterocyclyl, arylene, or heteroarylene is unsubstituted or substituted;
or a pharmaceutically acceptable salt thereof.

2. The compound of claim 1 having the structure 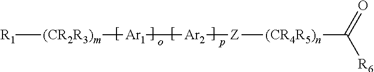
wherein
R₁ is H, halogen, —NR₇R₈, —NH—C(═O)—OR₇, —OR₇, —NO₂, —CN, —SR₇, —SO₂R₇, —CO₂R₇, CF₃, —SOR₇, —POR₇, —C(═S)R₇, —C(═O)—NR₇R₈, —C(═NR₇)R₈, —P(═O) (OR₇)(OR₈), —P(OR₇)(OR₈), —C(═S)R₇, C₁₋₅ alkyl, C₂₋₅ alkenyl, C₂₋₅ alkynyl, aryl, heteroaryl, or heterocyclyl,
wherein R₇ and R₈ are each, independently, H, C₁₋₅ alkyl, C₂₋₅ alkenyl, C₂₋₅ alkynyl, aryl, or heteroaryl;
R₂ and R₃ are each, independently, H, —CH₂—R₉, —C(═O)OR₉, —C(═O)NR₉R₁₀, or —C(═O)R₉,
wherein
R₉ and R₁₀ are each, independently, H, C₁₋₁₀ alkyl, C₂₋₁₀ alkenyl, C₂₋₁₀ alkynyl, —(CH₂)ᵣ—OR₁₈, 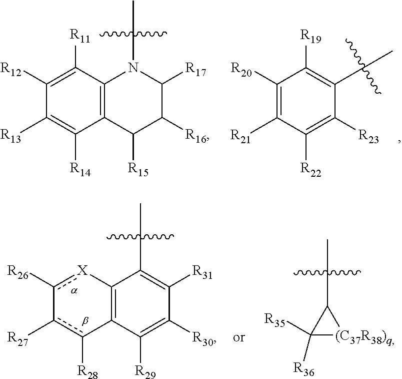
wherein
q is an integer from 1 to 6;
r is an integer from 1 to 10;
R₁₈ is H, C₁₋₁₀ alkyl, C₂₋₁₀ alkenyl, C₂₋₁₀ alkynyl, cycloalkyl, aryl, heteroaryl, or heterocyclyl;
bond α and bond β are each, independently, present or absent;
when bond α is present, X is N or CR₃₂;
when bond α is absent, X is NR₃₂ or CR₃₁R₃₂;
R₁₁, R₁₂, R₁₃, R₁₄, R₁₅, R₁₆, R₁₇, R₁₉, R₂₀, R₂₁, R₂₂, R₂₃, R₂₆, R₂₇, R₂₈, R₂₉, R₃₀, R₃₁, R₃₂, R₃₅, R₃₆, R₃₇, and R₃₈ are each, independently, H, halogen, —NO₂, —CN, —NR₂₄R₂₅, —SR₂₄, —SO₂R₂₄, —CO₂R₂₄, —OR₂₄, CF₃, —SOR₂₄, —POR₂₄, —C(═S)R₂₄, —C(═NR₂₄)R₂₅, —P(═O) (OR₂₄)(OR₂₅), —P(OR₂₄)(OR₂₅), —C(═S)R₂₄, C₁₋₁₀ alkyl, C₂₋₁₀ alkenyl, C₂₋₁₀ alkynyl, aryl, heteroaryl, or heterocyclyl;
wherein R₂₄ and R₂₅ are each, independently, H, C₁₋₁₀ alkyl, C₂₋₁₀ alkenyl, C₂₋₁₀ alkynyl, aryl, heteroaryl, or heterocyclyl;
wherein R₂ or R₃ is other than H;
m is an integer from 0 to 5;
Ar₁ and Ar₂ are each, independently, arylene or a heteroarylene other than isooxazolylene;
o and p are each, independently, 0 or 1;
Z is —NR₄₃—C(═O)—, or a bond,
wherein R₄₃ is H, —(CR₄₄R₄₅)ₛ—CR₄₄R₄₅R₄₆, C₁₋₁₀ alkyl substituted by heterocyclyl, C₂₋₁₀ alkenyl, C₂₋₁₀ alkynyl, aryl, heteroaryl, or heterocyclyl,
wherein
s is an integer from 1 to 10;
R₄₄, R₄₅, and R₄₆ are each, independently, H, halogen, —NO₂, —CN, —NR₄₇R₄₈, —SR₄₇, —SO₂R₄₇, —CO₂R₄₇, —OR₄₇, CF₃, —SOR₄₇, —POR₄₇, —C(═S)R₄₇, —C(═NR₄₇)R₄₈, —P(═O)(OR₄₇)(OR₄₈), —P(OR₄₇)(OR₄₈), —C(═S)R₄₇, C₁₋₁₀ alkyl, C₂₋₁₀ alkenyl, C₂₋₁₀ alkynyl, aryl, heteroaryl, or heterocyclyl,
wherein R₄₇ and R₄₈ are each, independently, H, C₁₋₁₀ alkyl, C₂₋₁₀ alkenyl, C₂₋₁₀ alkynyl, aryl, heteroaryl, or heterocyclyl;
wherein when Z is a bond, o is 0, p is 0, R₂ or R₃ is C(═O)NR₉R₁₀, then
R₉ and R₁₀ are each, independently, H, C₁₋₁₀ alkyl, C₂₋₁₀ alkenyl, C₂₋₁₀ alkynyl, —(CH₂)ᵣ—OR₁₈, 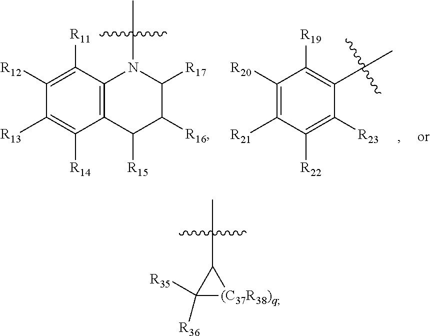
R₄ and R₅ are each, independently, H, C₁₋₁₀ alkyl, C₂₋₁₀ alkenyl, C₂₋₁₀ alkynyl, aryl, heteroaryl, or heterocyclyl;
n is an integer from 2 to 10;
R₆ is —OR₄₉ or —NH—OR₄₉,
wherein R₄₉ is H, C₁₋₁₀ alkyl, C₂₋₁₀ alkenyl, C₂₋₁₀ alkynyl, aryl, heteroaryl, or heterocyclyl;
wherein each occurrence of alkyl, alkenyl, or alkynyl is unsubstituted or substituted, branched or unbranched;
wherein each occurrence of cycloalkyl, aryl, heteroaryl, heterocyclyl, arylene, or heteroarylene is unsubstituted or substituted;
or a pharmaceutically acceptable salt thereof.

3. The compound of claim 2 having the structure 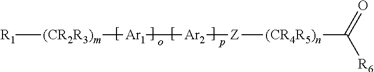
wherein
R₁ is H or —NH—C(═O)—OR₇,
wherein R₇ is C₁₋₅ alkyl;
R₂ and R₃ are each, independently, H, —CH₂—R₉, or —C(═O)NR₉R₁₀,
wherein
R₉ and R₁₀ are each, independently, H, C₁₋₁₀ alkyl, C₂₋₁₀ alkenyl, C₂₋₁₀ alkynyl, —(CH₂)ᵣ—OR₁₈, or 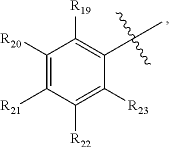
wherein
r is an integer from 1 to 10;
R₁₈ is H, C₁₋₁₀ alkyl, C₂₋₁₀ alkenyl, C₂₋₁₀ alkynyl, cycloalkyl, aryl, heteroaryl, or heterocyclyl;
R₁₉, R₂₀, R₂₁, R₂₂, R₂₃ are each, independently, H, halogen, —NO₂, —CN, —NR₂₄R₂₅, —SR₂₄, —SO₂R₂₄, —CO₂R₂₄, —OR₂₄, CF₃, —SOR₂₄, —POR₂₄, —C(═S)R₂₄, —C(═NR₂₄)R₂₅, —P(═O) (OR₂₄)(OR₂₅), —P(OR₂₄)(OR₂₅), —C(═S)R₂₄, C₁₋₁₀ alkyl, C₂₋₁₀ alkenyl, C₂₋₁₀ alkynyl, aryl, heteroaryl, or heterocyclyl;
wherein R₂ or R₃ is other than H;
m is an integer from 0 to 5;
Ar₁ and Ar₂ are each, independently, arylene or a heteroarylene other than isooxazolylene;
o and p are each, independently, 0 or 1;
Z is —NH—C(═O)—, or a bond,
wherein when Z is a bond, o is 0, p is 0, R₂ or R₃ is C(═O)NR₉R₁₀, then
R₉ and R₁₀ are each, independently, H, C₁₋₁₀ alkyl, C₂₋₁₀ alkenyl, C₂₋₁₀ alkynyl, —(CH₂)ᵣ—OR₁₈, 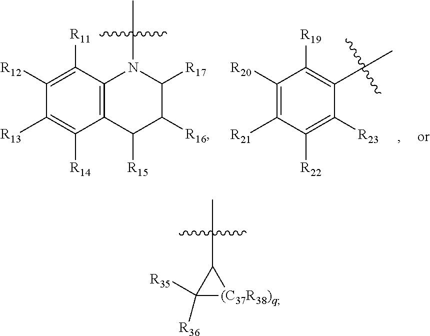
R₄ and R₅ are each H;
n is an integer from 2 to 10;
R₆ is —NH—OR₄₉,
wherein R₄₉ is H, C₁₋₁₀ alkyl, C₂₋₁₀ alkenyl, C₂₋₁₀ alkynyl, aryl, heteroaryl, or heterocyclyl;
wherein each occurrence of alkyl, alkenyl, or alkynyl is unsubstituted or substituted, branched or unbranched;
wherein each occurrence of cycloalkyl, aryl, heteroaryl, heterocyclyl, arylene, or heteroarylene is unsubstituted or substituted;
or a pharmaceutically acceptable salt thereof.

4. The compound of claim 3, wherein
R₁ is H or —NH—C(═O)—O-tert-butyl;
R₂ and R₃ are each, independently, H, —CH₂—R₉, or —C(═O)NR₉R₁₀,
wherein
R₉ and R₁₀ are each, independently, H or 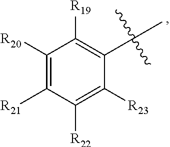
wherein R₁₉, R₂₀, R₂₁, R₂₂, R₂₃ are each, independently, H or tert-butyl;
wherein R₂ or R₃ is other than H;
m is an integer from 0 to 5;
Ar₁ and Ar₂ are each, independently, arylene or thiophenylene;
o and p are each, independently, 0 or 1;
Z is —NH—C(═O)—, or a bond,
wherein when Z is a bond, o is 0, p is 0, R₂ or R₃ is C(═O)NR₉R₁₀, then
R₉ and R₁₀ are each, independently, H or 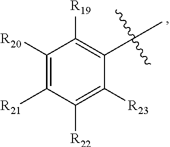
wherein R₁₉, R₂₀, R₂₁, R₂₂, R₂₃ are each, independently, H or tert-butyl;
R₄ and R₅ are each H;
n is an integer from 5 to 7;
R₆ is —NH—OH;
or a pharmaceutically acceptable salt thereof.

5. (canceled)

6. The compound of claim 1 having the structure 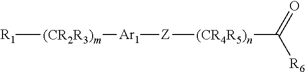
wherein
R₁ is H, halogen, —NR₇R₈, —NR₅₁—C(═O)—R₅₂, —OR₇, —NO₂, —CN, —SR₇, —SO₂R₇, —CO₂R₇, CF₃, —SOR₇, —POR₇, —C(═S)R₇, —C(═O)—NR₇R₈, —CH₂—C(═O)—NR₇R₈, —C(═NR₇)R₈, —P(═O)(OR₇)(OR₈), —P(OR₇)(OR₈), —C(═S)R₇, C₁₋₅ alkyl, C₂₋₅ alkenyl, C₂₋₅ alkynyl, aryl, heteroaryl, or heterocyclyl,
wherein R₇, R₈, R₅₁ and R₅₂ are each, independently, H, C₁₋₅ alkyl, C₂₋₅ alkenyl, C₂₋₅ alkynyl, aryl, or heteroaryl;
R₂ and R₃ are each, independently, H, —(NH), —C(═O)OR₉, —C(═O)NR₉R₁₀, or —C(═O)R₉,
wherein
R₉ and R₁₀ are each, independently, H, C₁₋₁₀ alkyl, C₂₋₁₀ alkenyl, C₂₋₁₀ alkynyl, —(CH₂)ᵣ—OR₁₈, 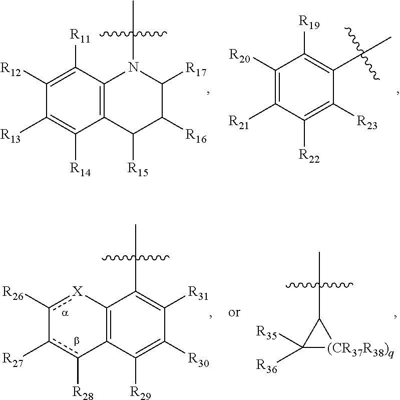
wherein
q is an integer from 1 to 6;
r is an integer from 1 to 10;
R₁₈ is H, C₁₋₁₀ alkyl, C₂₋₁₀ alkenyl, C₂₋₁₀ alkynyl, cycloalkyl, aryl, heteroaryl, or heterocyclyl;
bond α and bond β are each, independently, present or absent;
when bond α is present, X is N or CR₃₂;
when bond α is absent, X is NR₃₂ or CR₃₁R₃₂;
R₁₁, R₁₂, R₁₃, R₁₄, R₁₅, R₁₆, R₁₇, R₁₉, R₂₃, R₂₁, R₂₂, R₂₃ R₂₆, R₂₇, R₂₅, R₂₉, R₃₀, R₃₁, R₃₂, R₃₅, R₃₆, R₃₇, and R₃₄ are each, independently, H, halogen, —NO₂, —CN, —NR₂₄R₂₅, —SR₂₄, —SO₂R₂₄, —CO₂R₂₄, —OR₂₄, CF₃, —SOR₂₄, —POR₂₄, —C(═S)R₂₄, —C(═NR₂₄)R₂₅, —P(═O)(OR₂₄)(OR₂₅), —P(OR₂₄)(OR₂₅), —C(═S)R₂₄, C₁₋₁₀ alkyl, C₂₋₁₀ alkenyl, C₂₋₁₀ alkynyl, aryl, heteroaryl, or heterocyclyl;
wherein R₂₄ and R₂₅ are each, independently, H, C₁₋₁₀ alkyl, C₂₋₁₀ alkenyl, C₂₋₁₀ alkynyl, aryl, heteroaryl, or heterocyclyl;
wherein R₂ or R₃ is other than H;
Ar₁ is arylene or heteroarylene, wherein the heteroarylene is not isooxazolylene;
m is an integer from 0 to 5;
Z is —NR₄₃—C(═O)—, or —C(═O)—NR₅₀—
wherein R₄₃ and R₅₀ are, independently, H, —(CR₄₄R₄₅)ₛ—CR₄₄R₄₅R₄₆, C₁₋₁₀ alkyl substituted by heterocyclyl, C₂₋₁₀ alkenyl, C₂₋₁₀ alkynyl, aryl, heteroaryl, or heterocyclyl,
wherein
s is an integer from 1 to 10;
R₄₄, R₄₅, and R₄₆ are each, independently, H, halogen, —NO₂, —CN, —NR₄₇R₄₈, —SR₄₇, —SO₂R₄₇, —CO₂R₄₇, —OR₄₇, CF₃, —SOR₄₇, —POR₄₇, —C(═S)R₄₇, —C(═NR₄₇)R₄₈, —P(═O)(OR₄₇)(OR₄₈), —P(OR₄₇)(OR₄₈), —C(═S)R₄₇, C₁₋₁₀ alkyl, C₂₋₁₀ alkenyl, C₂₋₁₀ alkynyl, aryl, heteroaryl, or heterocyclyl,
wherein R₄₇ and R₄₈ are each, independently, H, C₁₋₁₀ alkyl, C₂₋₁₀ alkenyl, C₂₋₁₀ alkynyl, aryl, heteroaryl, or heterocyclyl;
R₄ and R₅ are each, independently, H, C₁₋₁₀ alkyl, C₂₋₁₀alkenyl, C₂₋₁₀ alkynyl, aryl, heteroaryl, or heterocyclyl;
n is an integer and is 0 or from 2 to 10;
R₆ is —OR₄₉ or —NH—OR₄₉,
wherein R₄₉ is H, C₁₋₁₀ alkyl, C₂₋₁₀ alkenyl, C₂₋₁₀ alkynyl, aryl, heteroaryl, or heterocyclyl;
wherein each occurrence of alkyl, alkenyl, or alkynyl is unsubstituted or substituted, branched or unbranched;
wherein each occurrence of cycloalkyl, aryl, heteroaryl, heterocyclyl, arylene, or heteroarylene is unsubstituted or substituted;
or a pharmaceutically acceptable salt thereof.

7. The compound of claim 6, wherein R₁ is H, F, —NH₂, —OH, —CH₃, —NR₅₁—C(═O)—R₅₂, —CH₂—C(═O)—NR₇R₈, or —(C═O)—NR₇R₈
wherein R₇ is H, —C₂H₄OH, —CH₂—CHOH—CH₂OH, or aryl,
wherein R₅₁ is H, —C₂H₄OH, or —CH₂—CHOH—CH₂OH,
wherein R₈ and R₅₂ are, independently, a fluorine-substituted aryl, quinolinyl, or a nitrogen-containing heteroaryl having the structure: 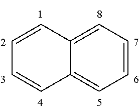
wherein the point of attachment is any one of atom positions 1, 2, 3, 4, 5, 6, 7, or 8, and wherein the nitrogen atom can be at any of atom positions 1, 2, 3, 4, 5, 6, 7, or 8, with the proviso that the point of attachment and the nitrogen atom are not at the same atom position,
or a heteroaryl comprising two nitrogen atoms having the structure: 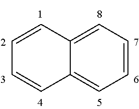
wherein the point of attachment is any one of atom positions 1, 2, 3, 4, 5, 6, 7, or 8, and wherein a first nitrogen atom can be at any of atom positions 1, 2, 3, 4, 5, 6, 7, or 8, and wherein a second nitrogen atom is at any of atom positions 1, 2, 3, 4, 5, 6, 7, or 8, with the provisos that (a) no nitrogen atom is directly bound to another nitrogen atom and (b) the point of attachment, and the first nitrogen atom, and the second nitrogen atom are each at different atom positions,
or a pharmaceutically acceptable salt thereof.

8. The compound of claim 6,
wherein
R₁ is H, halogen, —NR₇R₈, —OR₇, —(C═O)—NR₇R₈, C₁₋₅ alkyl or NR₅₁—C(═O)—NR₅₂,
wherein R₇ and R₈ are each, independently, H or heteroaryl and wherein R₅₁ is H or a C₁-C₅ alkyl and R₅₂ is heteroaryl;
or a pharmaceutically acceptable salt thereof.

9. The compound of claim 6, wherein Z is —C(═O)—NR₅₀—,
wherein R₅₀ is H or a C₁-C₅ alkyl,
or a pharmaceutically acceptable salt thereof.

10. (canceled)

11. The compound of claim 6, wherein
R₂ and R₃ are each, independently, H, —C(═O)OR₉, —C(═O)NR₉R₁₀, or —C(═O)R₉,
wherein
R₉ and R₁₀ are each, independently, H, C₁₋₁₀ alkyl, —(CH₂)ᵣ—OR₁₈, 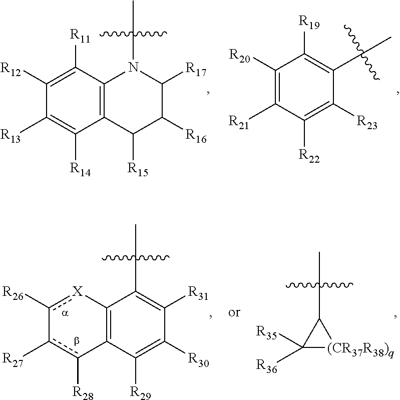
wherein
q is an integer from 1 to 6;
r is an integer from 1 to 10;
R₁₈ is H, C₁₋₁₀ alkyl, C₂₋₁₀ alkenyl, C₂₋₁₀ alkynyl, cycloalkyl, aryl, heteroaryl, or heterocyclyl;
bond α and bond β are each, independently, present or absent;
when bond α is present, X is N or CR₃₂;
when bond α is absent, X is NR₃₂ or CR₃₁R₃₂;
R₁₁, R₁₂, R₁₃, R₁₄, R₁₅, R₁₆, R₁₇, R₁₉, R₂₀, R₂₁, R₂₂, R₂₃ R₂₆, R₂₇, R₂₈, R₂₉, R₃₀, R₃₁, R₃₂, R₃₅, R₃₆, R₃₇, and R₃₈ are each, independently, H, halogen, —NO₂, —CN, —NR₂₄R₂₅, —SR₂₄, —SO₂R₂₄, —CO₂R₂₄, —OR₂₄, CF₃, —SOR₂₄, —POR₂₄, —C(═S)R₂₄, —C(═NR₂₄)R₂₅, —P(═O)(OR₂₄)(OR₂₅), —P(OR₂₄)(OR₂₅), —C(═S)R₂₄, C₁₋₁₀ alkyl, C₂₋₁₀ alkenyl, C₂₋₁₀ alkynyl, aryl, heteroaryl, or heterocyclyl;
wherein R₂₄ and R₂₅ are each, independently, H, C₁₋₁₀ alkyl, C₂₋₁₀ alkenyl, C₂₋₁₀ alkynyl, aryl, heteroaryl, or heterocyclyl;
wherein R₂ or R₃ is other than H;
or a pharmaceutically acceptable salt thereof.

12. (canceled)

13. The compound of claim 8 having the structure 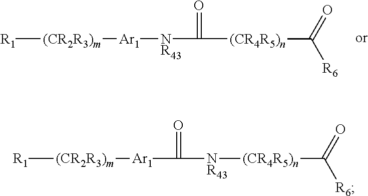
or a pharmaceutically acceptable salt thereof.

14. (canceled)

15. The compound of claim 11, wherein m is an integer from 0 to 2 and n is an integer from 3 to 8;
or a pharmaceutically acceptable salt thereof.

16. (canceled)

17. The compound of claim 15,
wherein
R₆ is —OR₄₉ or —NH—OR₄₉,
wherein R₄₉ is H or C₁₋₁₀ alkyl;
or a pharmaceutically acceptable salt thereof.

18. The compound of claim 17 having the structure 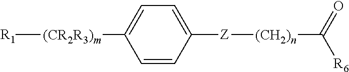
wherein
R₁ is H, F, —NH₂, —OH, —CH₃, or —(C═O)—NH—R₈,
wherein R₈ is quinolinyl,
R₂ and R₃ are each, independently, H, —C(═O)OR₉, —C(═O)NR₉R₁₀, or —C(═O)R₉,
wherein
R₉ and R₁₀ are each, independently, H, tert-butyl, neopentyl, —(CH₂)₂—OH, 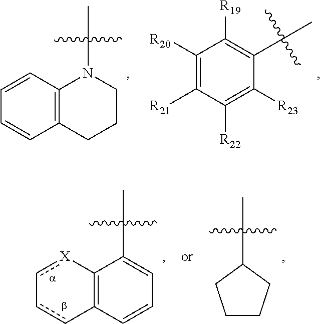
wherein R₁₉, R₂₀, R₂₁, R₂₂, R₂₃ are each, independently, H, tert-butyl, or F;
bond α and bond β are each, independently, present or absent;
when bond α is present, bond β is present and X is N or CH;
when bond α is absent, bond β is absent and X is CH₂;
wherein R₂ or R₃ is other than H;
m is 0 or 1;
Z is —NR₄₃—C(═O)—,
wherein R₄₃ is H, —CH₂—CH(OH)—CH₂(OH), or 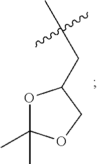 [SMILES: *CC1COC(C)(C)O1]
n is an integer from 5 to 7;
R₆ is —OR₄₉ or —NH—OR₄₉,
wherein R₄₉ is H, —CH₃, —CH₂CH₃, or tert-butyl;
or a pharmaceutically acceptable salt thereof.

19. The compound of claim 7 having the structure 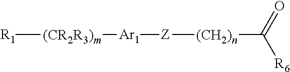
wherein
m is 0 or 1;
n is 0 or an integer from 5 to 7;
Ar₁ is 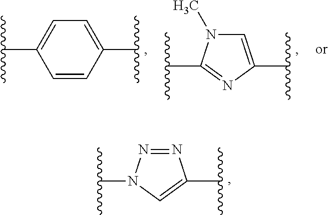
R₁ is H, F, —NH₂, —OH, —CH₃, —NR₅₁—C(═O)—R₅₂, —CH —C(═O)—NR₇R₈, or —(C═O)—NR₇R₈
wherein R₇ is H, —C₂H₄OH, —CH—CHOH—CH₂OH, or aryl,
wherein R₅₁ is H, —C₂H₄OH, or —CH₂—CHOH—CH₂OH,
wherein R₈ and R₅₂ are, independently, a fluorine-substituted aryl, quinolinyl, or a nitrogen-containing heteroaryl having the structure: 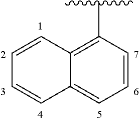 [SMILES: *c1cccc2ccccc12]
wherein the nitrogen atom can be at any of positions 2, 3, 4, 5, 6, or 7, or a heteroaryl comprising two nitrogen atoms having the structure: 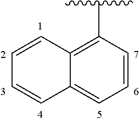 [SMILES: *c1cccc2ccccc12]
wherein one nitrogen atom is at position 1, 2, 3, 4, 5, 6, or 7 and the second nitrogen atom is in any one of the remaining numbered positions, with the proviso that no nitrogen atom is directly bound to another nitrogen atom,
R₂ and R₃ are, if present, each, independently, H, —(NH₂), —C(═O)OR₉, —C(═O)NR₉R₁₀ or —C(═O)R₉,
wherein
R₉ and R₁₀ are each, independently, H, tert-butyl, neopentyl, —(CH₂)₂—OH, 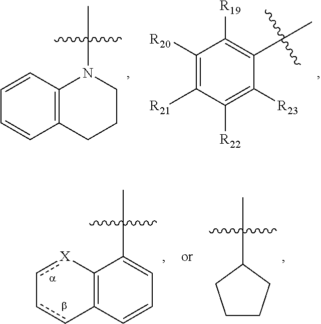
wherein R₁₉, R₂₀, R₂₁, R₂₂, R₂₃ are each, independently, H, tert-butyl, or F;
bond α and bond β are each, independently, present or absent;
when bond α is present, bond β is present and X is N or CH;
when bond α is absent, bond β is absent and X is CH₂;
wherein R₂ or R₃ is other than H;
wherein Z is —NR₄₃—C(═O)—, or is —C(═O)—NR₅₀—, wherein R₅₀ is H,
wherein R₄₃ is H, —CH₂—CH(OH)—CH₂(OH), or 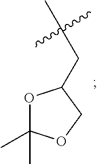
wherein R₆ is —OR₄₉ or —NH—OR₄₉,
wherein R₄₉ is H, —CH₃, —CH₂CH₃, or tert-butyl;
or a pharmaceutically acceptable salt thereof.

20-23. (canceled)

24. The compound of claim 18 having the structure 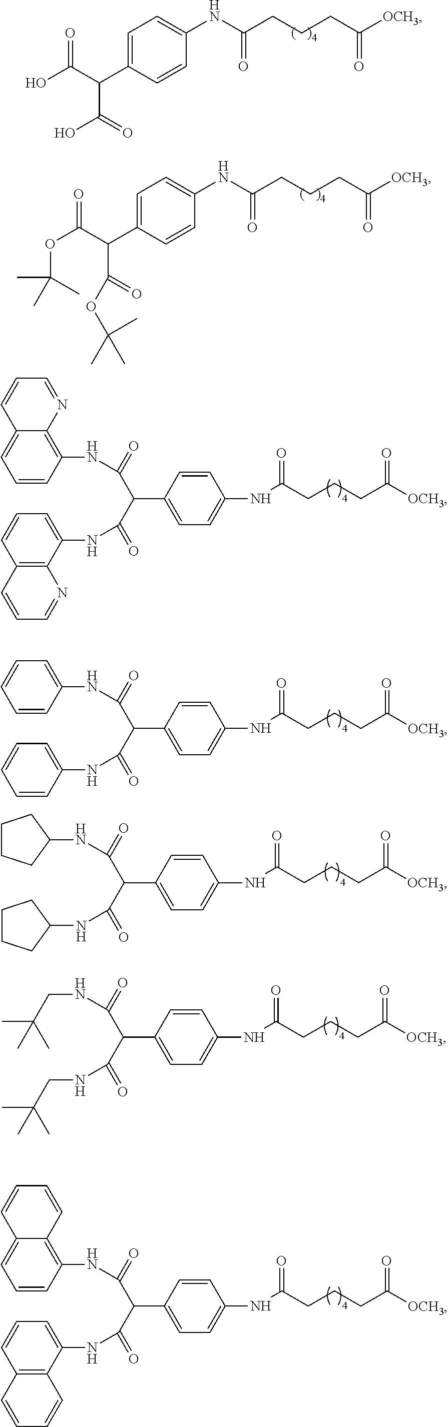 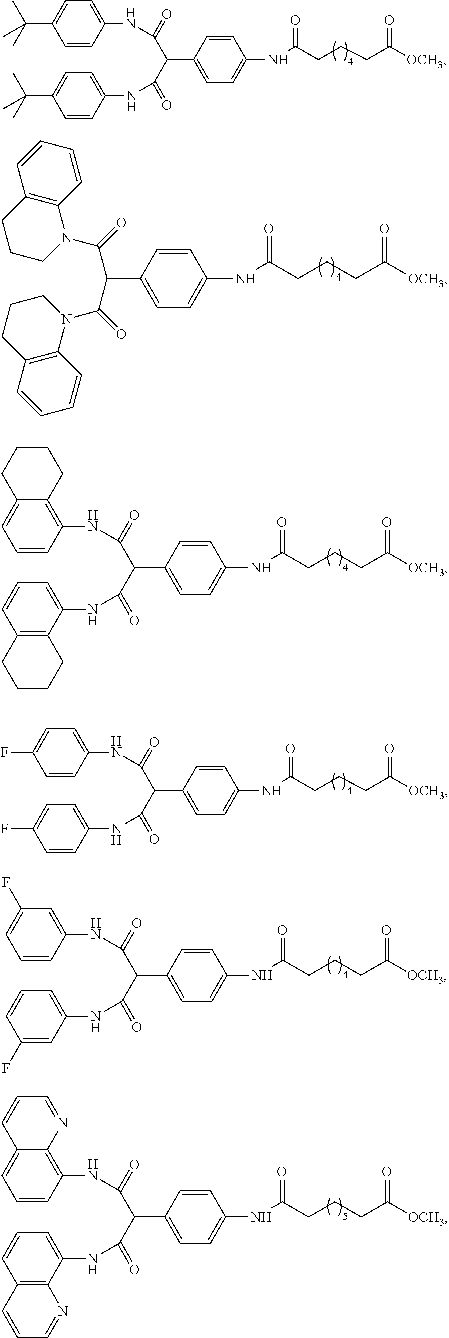 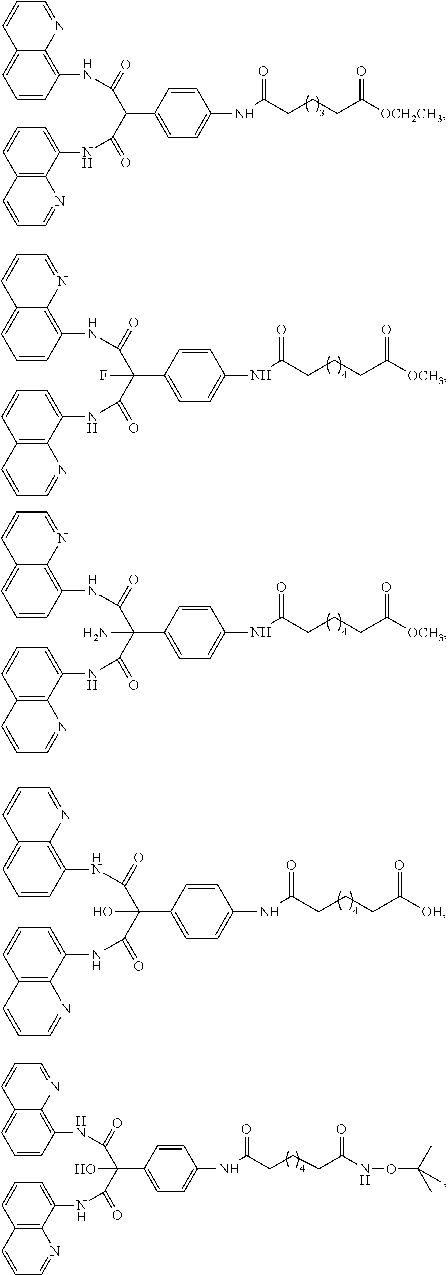 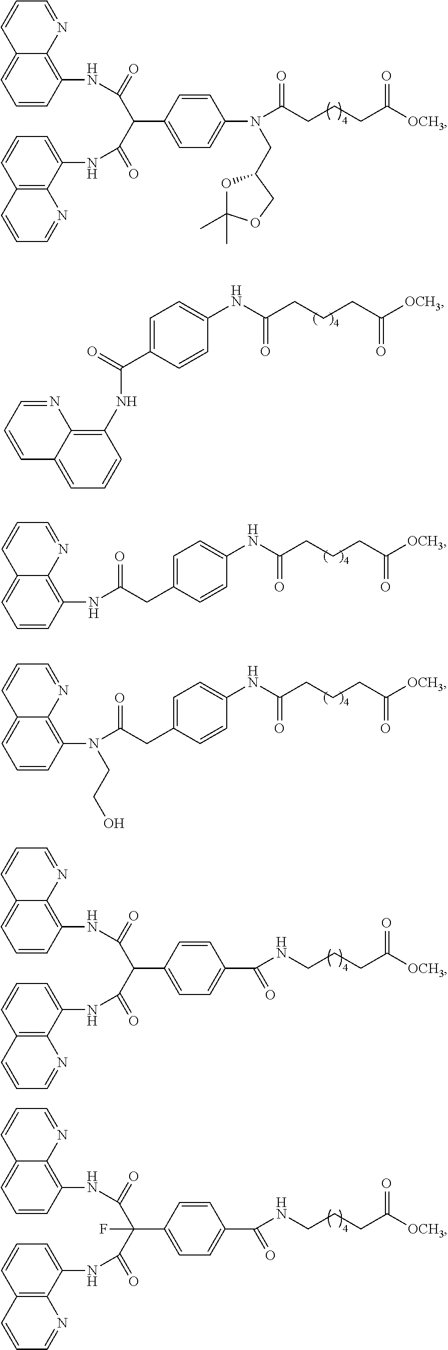 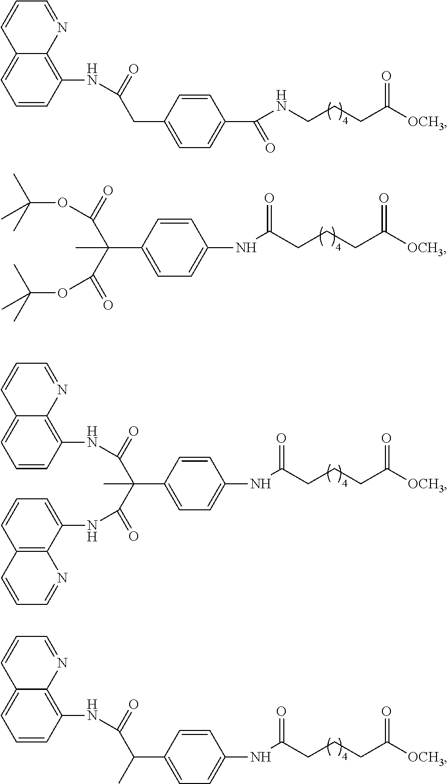
or a pharmaceutically acceptable salt thereof.

25. The compound of claim 18 having the structure 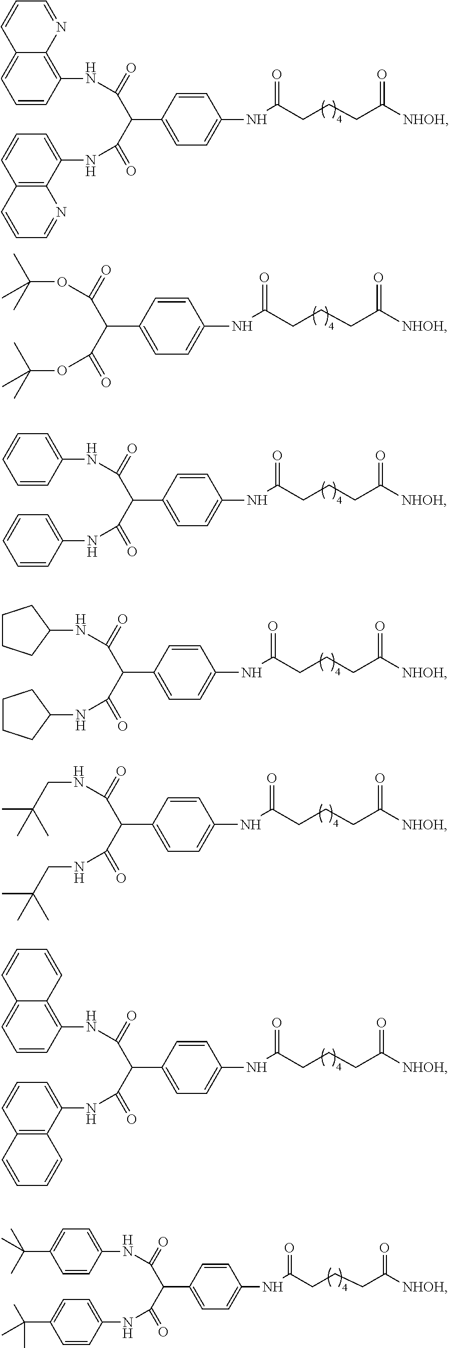 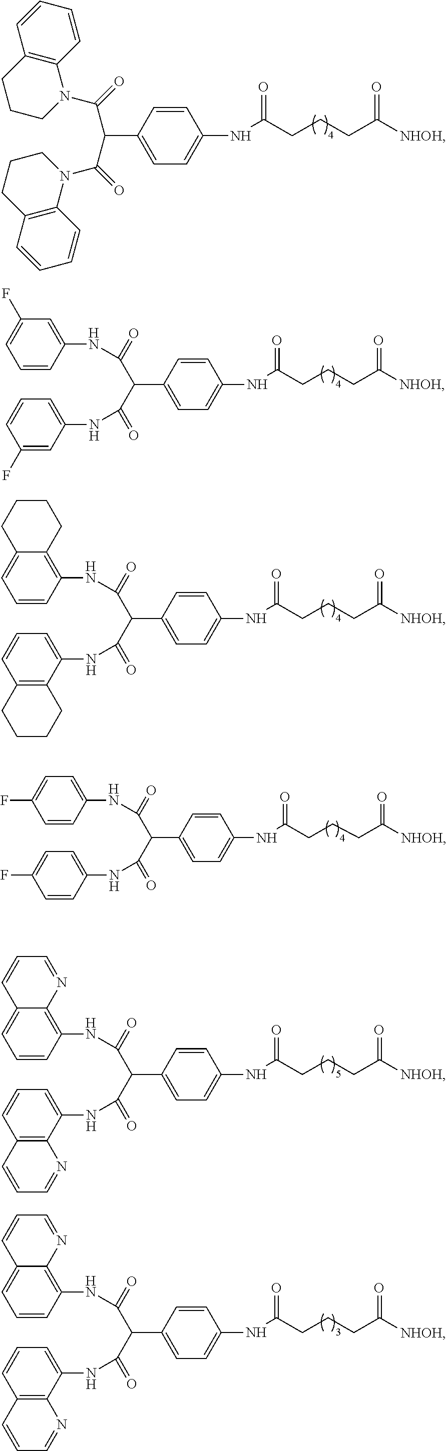 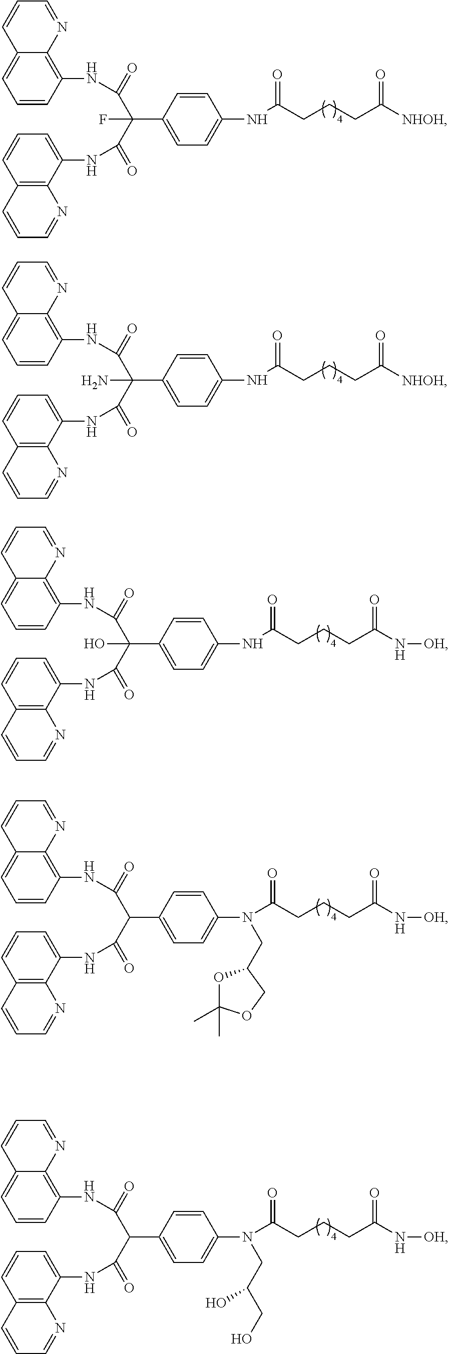 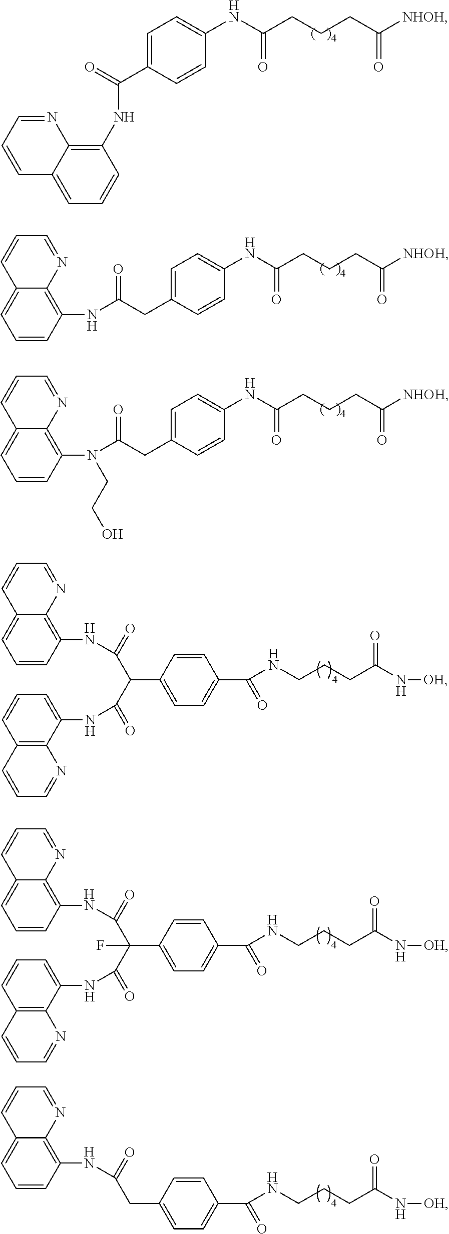 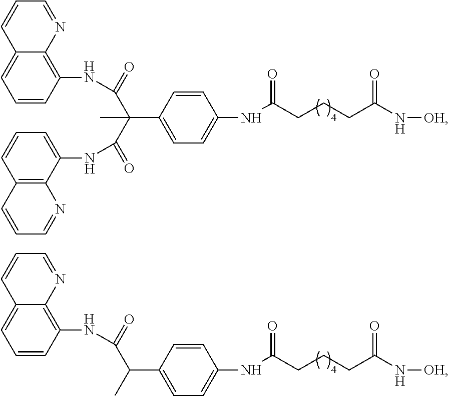
or a pharmaceutically acceptable salt thereof.

26. (canceled)

27. The compound of claim 7 having the structure: 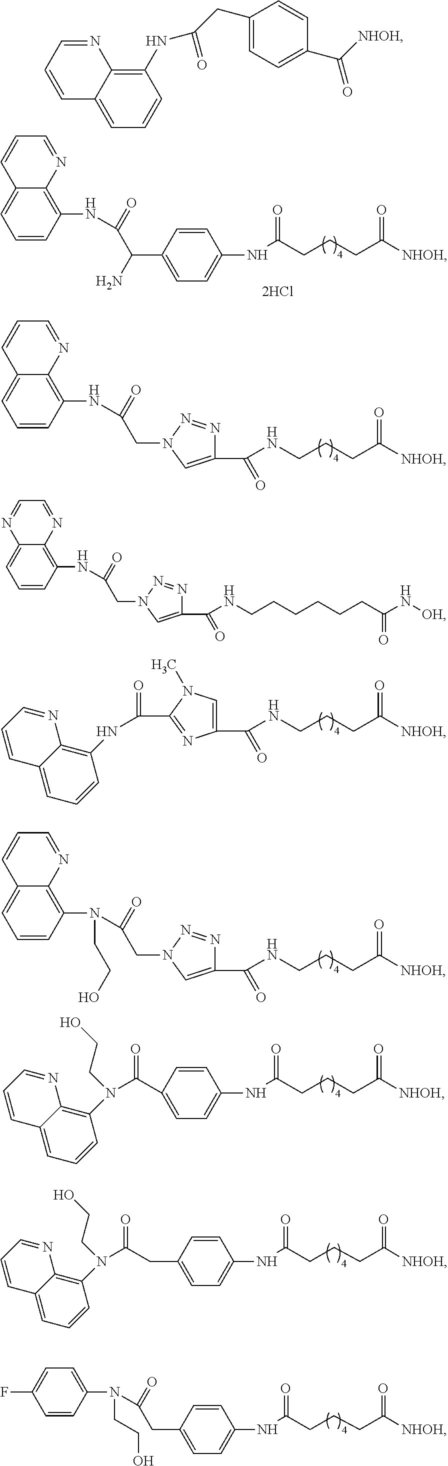 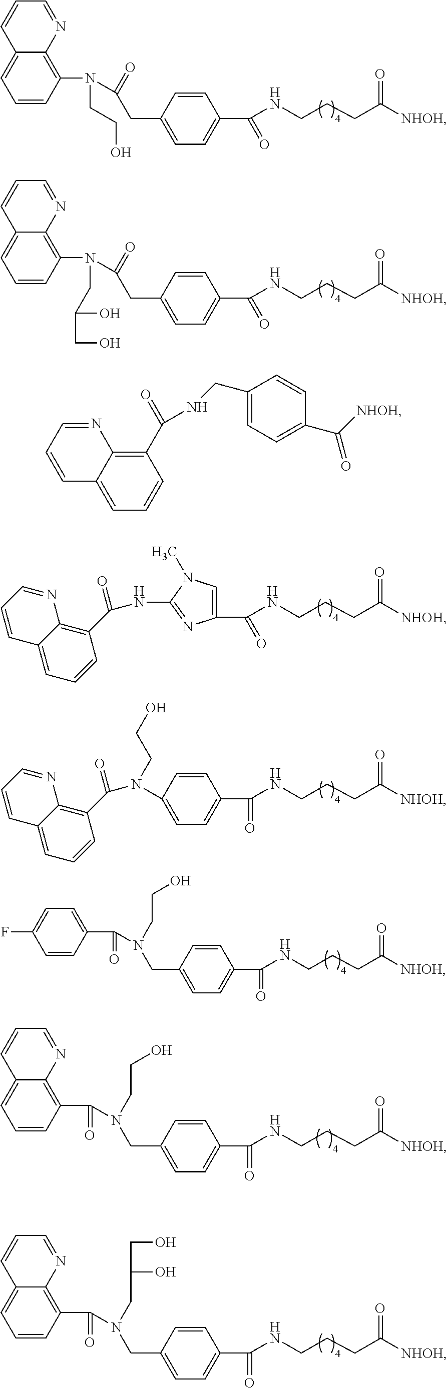 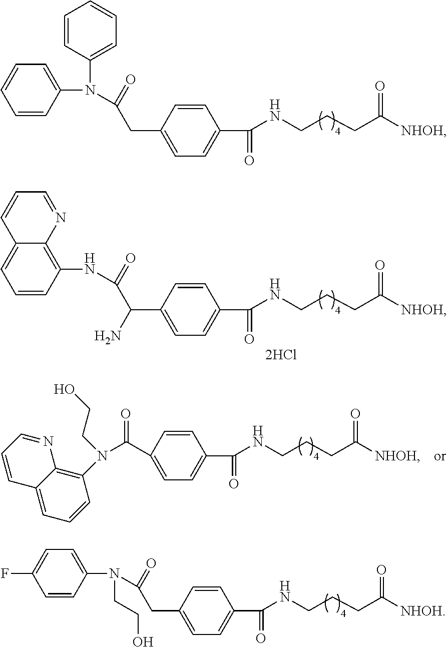
or a pharmaceutically acceptable salt thereof.

28. (canceled)

29. The compound of claim 1, having the structure: 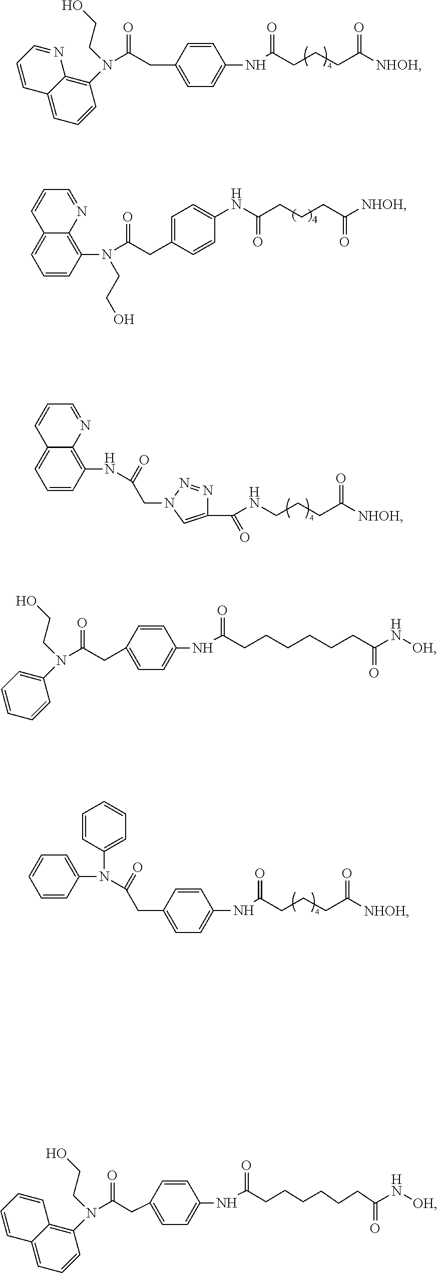 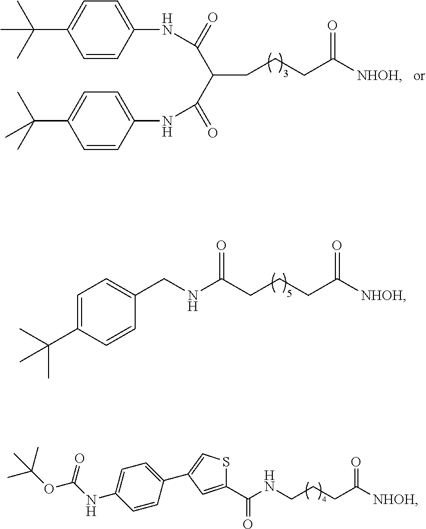
or a pharmaceutically acceptable salt thereof.

30-31. (canceled)

32. A pharmaceutical composition comprising the compound of any one of claim 1 and a pharmaceutically acceptable carrier.

33. A method of inhibiting the activity of a histone deacetylase in a cell comprising contacting the histone deacetylase with the compound of claim 1 so as to inhibit the activity of the histone deacetylase, preferably HDAC6.

34-40. (canceled)
